(19) United States
(12) Patent Application Publication
(10) Pub. No.: US 2004/0043995 A1
(43) Pub. Date: Mar. 4, 2004

(54) Novel triazole derivatives, process for their preparation and pharmaceutical compositions containing them

(76) Inventors: Eric Bignon, Pinsaguel (FR); Jean Pierre Bras, Toulouse (FR); Paul De Cointet, Toulouse (FR); Pierre Despeyroux, Labarthe sur Leze (FR); Daniel Frehel, Estadens (FR); Danielle Gully, Muret (FR)

Correspondence Address: SANOFI-SYNTHELABO INC., 9 GREAT VALLEY PARKWAY, P.O. BOX 3026, MALVERN, PA, 19355, US

(21) Appl. No.: 10/317,583

(22) Filed: Dec. 12, 2002

Related U.S. Application Data
(63) Continuation of application No. 09/423,505, filed on Nov. 9, 1999, now abandoned.
Application No. 09/423,505 is a 371 of international application No. PCT/FR98/00905, filed on May 6, 1998.

(30) Foreign Application Priority Data
May 13, 1997 (FR) 97 05850

(51) Int. Cl. C07D 413/14; C 07D 4 3/14; A61K 31/5377; A61K 31/496; A61K 31/4196; A61K 31/454

(52) U.S. Cl. 514/235.8; 514/383; 514/254.05; 514/323; 514/381; 544/132; 544/366; 546/201; 548/254; 548/264.8

(57) ABSTRACT

The invention relates to a compound of formula

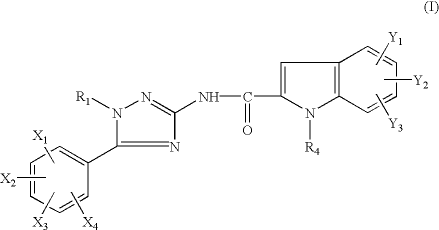

in which R₁, X₁, X₂, X₃, X₄, R₄, Y₁, Y₂ and Y₃ are as defined in claim 1.

These compounds are CCK-receptor agonists.

The present invention relates to novel triazole derivatives, to a process for their preparation and to medicines containing them.

More particularly, the present invention concerns novel non-peptide compounds displaying affinity for cholecystokinin (CCK) receptors.

CCK is a peptide which, in response to an ingestion of food, is secreted peripherally and participates in regulating many digestive processes (Crawley J. N. et al., Peptides, 1994, 15 (4), 731-735).

CCK has since been identified in the brain, and might be the most abundant neuropeptide acting as a neuromodulator of cerebral functions by stimulation of CCK-B type receptors (Crawley J. N. et al., Peptides, 1994, 15 (4), 731-735). In the central nervous system, CCK interacts with dopamine-mediated neuronal transmission (Crawley J. N. et al., ISIS Atlas of Sci., Pharmac. 1988, 84-90). It also plays a role in mechanisms involving acetylcholine, gaba (4-aminobutyric acid), serotonin, opioides, somatostatin, and substance P and in ion channels.

Its administration brings about physiological changes: palpebral ptosis, hypothermia, hyperglycaemia, catalepsy; and behaviour changes, hypolocomotion, decrease in exploratory ability, analgesia, a change in the learning faculty and a change in sexual behaviour and satiety.

CCK exerts its biological activity via at least two types of receptors: CCK-A receptors located mainly peripherally, and CCK-B receptors essentially present in the cerebral cortex. The CCK-A receptors of peripheral type are also present in certain zones of the central nervous system, including the postrema area, the tractus solitarius nucleus and the interpedoncular nucleus (Moran T. H. et al., Brain Research, 1986, 362, 175-179; Hill D. R. et al., J. Neurosci. 1990, 10, 1070-1081; with, however, specific differences (Hill D. R. et al., J. Neurosci. 1990, 10, 1070-1081); Mailleux P. et al., Neurosci. Lett., 1990, 117, 243-247; Barrett R. W. et al., Mol. Pharmacol., 1989, 36, 285-290; Mercer J. G. et al., Neurosci Lett., 1992, 137, 229-231; Moran T. H. et al., Trends in Pharmacol. Sci., 1991, 12, 232-236).

At the periphery, via the CCK-A receptors (Moran T. H. et al., Brain Research, 1986, 362, 175-179), CCK delays gastric emptying, modifies intestinal motility, stimulates gallblader contraction, increases bile secretion and controls pancreatic secretion (McHugh P. R. et al., Fed. Proc., 1986, 45, 1384-1390; Pendleton R. G. et al., J. Pharmacol. Exp. Ther., 1987, 241, 110-116).

CCK may act in certain cases on the arterial pressure and have an influence on immune systems.

The role of CCK in the satiety signal is supported by the fact that the plasmatic concentrations of CCK, which are dependent on the composition of the meals (high concentrations of proteins or lipids) are, after meals, higher than those observed before meals (Izzo R. S. et al., Regul. Pept., 1984, 9, 21-34; Pfeiffer A. et al., Eur. J. Clin. Invest., 1993, 23, 57-62; Lieverse R. J. Gut, 1994, 35, 531). In bulimia sufferers, there is a decrease in the secretion of CCK induced by a meal, (Geraciotti T. D. Jr. et al., N. Engl. J. Med., 1988, 319, 683-688; Devlin M. J. et al., Am. J. Clin. Nutr., 1997, 65, 114-120) and a lowering of the CCK concentrations in the cerebrospinal fluid (Lydiard R. B. et al., Am. J. Psychiatry, 1993, 150, 1099-1101). In the T lymphocytes, which is a cell compartment that may reflect central neuronal secretions, the basal CCK concentrations are significantly lower in patients suffering from bulimia nervosa (Brambilla F. et al., Psychiatry Research, 1995, 37, 51-56).

Treatments (for example with L-phenylalanine, or trypsin inhibitors) which increase the secretion of endogenous CCK give rise to a reduction in feeding in several species, including man (Hill A. J. et al., Physiol. Behav. 1990, 48, 241-246: Ballinger A B administration of exogenous CCK reduces feeding in many species, including man (Crawley J. N. et al. Peptides 1994, 15, 731-755).

The inhibition of feeding by CCK is mediated by the CCK-A receptor. Devazepide, an antagonist which is selective for the CCK-A receptors, inhibits the anorexigenic effect of CCK, whereas the selective agonists of these receptors inhibit feeding (Asin K. E. et al., Pharmacol. Biochem. Behav. 1992, 42, 699-704; Elliott R. L. et al., J. Med. Chem. 1994, 37, 309-313; Elliott R. L. et al., J. Med. Chem. 1994, 37, 1562-1568). Furthermore, OLEFT rats, which do not express the CCK-A receptor, are insensitive to the anorexigenic effect of CCK (Miyasaka K. et al., 1994, 180, 143-146).

Based on these lines of evidence of the key role of CCK in the peripheral satiety signal, the use of CCK agonists and antagonists as medicines in the treatment of certain eating behaviour disorders, obesity and diabetes is indisputable. A CCK-receptor agonist can also be used therapeutically in the treatment of emotional and sexual behaviour disorders and memory disorders (Itoh S. et al., Drug. Develop. Res., 1990, 21, 257-276), schizophrenia, psychosis (Crawley J. N. et al., Isis Atlas of Sci., Pharmac., 1988, 84-90 and Crawley J. N. Trends in Pharmacol. Sci., 1991, 12, 232-265), Parkinson's disease (Bednar I. et al., Biogenic amine, 1996, 12 (4), 275-284), tardive dyskinesia (Nishikawa T. et al., Prog. Neuropsychopharmacol. Biol. Psych., 1988, 12, 803-812; Kampen J. V. et al., Eur. J. Pharmacol., 1996, 298, 7-15) and various disorders of the gastrointestinal sphere (Drugs of the Future, 1992, 17 (3), 197-206).

CCK-A receptor agonists of CCK are described in the literature. For example, certain products having such properties are described in EP 383,690 and WO 90/06937, WO 95/28419, WO 96/11701 or WO 96/11940.

Most of the CCK-A agonists described to date are of peptide nature. Thus, FPL 14294 derived from CCK-7 is a powerful, unselective CCK-A agonist towards CCK-B receptors. It has powerful inhibitory activity on feeding in rats and in dogs after intranasal administration (Simmons R. D. et al., Pharmacol. Biochem. Behav., 1994, 47 (3), 701-708; Kaiser E. F. et al., Faseb, 1991, 5, A864). Similarly, it has been shown that A-71623, a tetrapeptide agonist which is selective for CCK-A receptors, is effective in models of anorexia over a period of 11 days and leads to a significant reduction in weight gain when compared with the control in rodents and cynomologous monkeys (Asin K. E. et al., Pharmacol. Biochem. Behav., 1992, 42, 699-704). Similarly, structural analogues of A 71623, which have good efficacy and selectivity for CCK-A receptors, have powerful anorexigenic activity in rats (Elliott R. L. et al., J. Med. Chem., 1994, 37, 309-313; Elliott R. L. et al., J. Med. Chem., 1994, 37, 1562-1568). GW 7854 (Hirst G. C. et al., J. Med. Chem., 1996, 38, 5236-5245), a-1,5-benzodiazepine, is an in vitro CCK-A receptor agonist. This molecule is also active orally on the contraction of the gallblader in mice and on feeding in rats.

It has now been found, surprisingly, that a series of triazole derivatives has partial or total agonist activity towards CCK-A receptors.

The compounds according to the invention underwent systematic studies in order to characterize:

their ability to displace [¹²⁵I]-CCK from its binding sites present on rat pancreatic membranes (CCK-A receptor) or 3T3 cells which express the human CCK-A recombinant receptor;

their affinity towards the CCK-B receptor, present on guinea pig cortex membranes, some of the compounds being selective or unselective CCK-A receptor ligands;

their CCK-A receptor agonist property by means of their capacity to: induce in vitro a mobilization of intracellular calcium in 3T3 cells which express human CCK-A receptor.

The triazole derivatives according to the present invention are CCK-A agonists since they are capable of stimulating partially, or totally like CCK, the mobilization of intracellular calcium in a cell D line which expresses human CCK-A recombinant receptor. They are, surprisingly, much more powerful than the thiazole derivatives described in patent applications EP 518,731 and EP 611,766, than the thiadiazole derivatives described in patent application EP 620,221, or than the benzodiazepin derivatives described in patent EP 667,344.

The reason for this is that these thiazole, thiadiazole and benzodiazepine derivatives are incapable of inducing this mobilization of intracellular calcium mediated by the CCK-A receptor.

The triazole derivatives according to the invention are also much more powerful than these thiazole, thiadiazole or benzodiazepine derivatives by virtue of their capacity to block in vivo, via the intraperitoneal route, gastric emptying in mice.

Thus, the CCK-A agonist properties were studied in vivo, by assessing their capacity to block gastric emptying in mice or to bring about, again in vivo, emptying of the gallblader in mice.

Certain derivatives also have CCK-B receptor antagonist activity.

Thus, the present invention relates to compounds of formula:

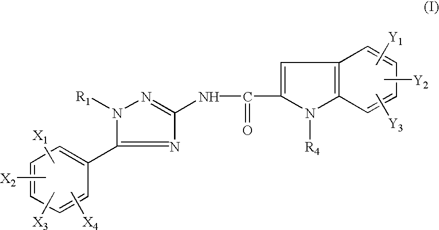

in which:

R₁ represents a (C₂-C₆)alkyl; a group —(CH₂)ₙ-G with n ranging from 0 to 5 and G representing a non-aromatic C₃-C₁₃ mono- or polycyclic hydrocarbon group optionally substituted with one or more (C₁-C₃)alkyl; a phenyl(C₁-C₃)alkyl in which the phenyl group is optionally substituted one or more times with a halogen, with a (C₁-C₃)alkyl or with a (C₁-C₃) alkoxy; a group —(CH₂)ₙNR₂R₃ in which n represents an integer from 1 to 6 and R₂ and R₃, which may be identical or different, represent a (C₁-C₃)alkyl or constitute, with the nitrogen atom to which they are attached, a morpholino, piperidino, pyrrolidinyl or piperazinyl group;

X₁, X₂, X₃ or X₄ each independently represents a hydrogen or halogen atom, a (C₁-C₆)alkyl, a (C₁-C₃)alkoxy or a trifluoromethyl; it being understood that only one from among X₁, X₂, X₃ and X₄ possibly represents a hydrogen atom;

R₄ represents hydrogen, a group —(CH₂) COOR₅ in which n is as defined above and R₅ represents a hydrogen atom, a (C₁-C₆)alkyl or a (C₆-C₁₀)aryl-(C₁-C₆)alkyl; a (C₁-C₆)alkyl; a group —(CH₂)ₙOR₅ or a group —(CH₂)ₙNR₂R₃ in which n, R₂, R₃ and R₅ are as defined above; a group —(CH₂)ₙ-tetrazolyl in which n is as defined above,

or R₄ represents one of these groups in the form of an alkali-metal or alkaline-earth metal salt;

Y₁, Y₂ and Y₃ independently represent a hydrogen, a halogen, a (C₁-C₃)alkyl, a (C₁-C₃)alkoxy, a nitro, cyano, (C₁-C₆)acylamino, carbamoyl, trifluoromethyl, a group COOR₆ in which R₆ represents hydrogen, or (C₁-C₃)alkyl;

or one of the salts or solvates thereof.

According to the present invention, “(C₁-C₆)alkyl” or “(C₂-C₆)alkyl” is understood to mean a straight or branched alkyl having 1 to 6 carbon atoms or 2 to 6 carbon atoms respectively.

The alkoxy radical denotes an alkyloxy radical in which alkyl is as defined above.

The acyl radical denotes an alkyl carbonyl radical in which alkyl is as defined above. (C₁-C₆)acylamino is a (C₁-C₆)alkylcarbonylamino.

The non-aromatic C₃-C₁₃ hydrocarbon groups 3 comprise saturated or unsaturated, fused or bridged, mono- or polycyclic radicals, which may be terpenic. These radicals are optionally mono- or polysubstituted with a (C₁-C₃)alkyl. The monocyclic radicals include cycloalkyls, for example cyclopropyl, cyclobutyl, cyclopentyl, cyclohexyl, cycloheptyl, cyclooctyl and cyclododecyl. The polycyclic radicals include, for example, norbornane, adamantane, hexahydroindane, norbornene, dihydrophenalene, bicyclo[2.2.1]heptane, bicyclo[3.3.1]nonane and tricyclo[5.2.1.0^{2.6}]decane.

According to the present invention, the term halogen is understood to mean an atom chosen from fluorine, chlorine, bromine and iodine, preferably fluorine or chlorine.

Examples of aryl groups are phenyl and naphthyl.

The alkali-metal or alkaline-earth metal cations are preferably chosen from those of sodium, potassium and calcium.

When a compound according to the invention has one or more asymmetric carbons, the optical isomers of this compound form an integral part of the invention.

When a compound according to the invention has stereoisomerism, for example of axial-equatorial type, the invention comprises all the stereoisomers of this compound. The salts of the compounds of formula (I) according to the present invention comprise those with inorganic or organic acids which allow a suitable separation or crystallization of the compounds of formula (I), such as picric acid, oxalic acid or an optically active acid, for example a tartaric acid, a dibenzoyltartaric acid, a mandelic acid or a camphorsulphonic acid, and those which form physiologically acceptable salts, such as the hydrochloride, hydrobromide, sulphate, hydrogensulphate, dihydrogenphosphate, maleate, fumarate, 2-naphthalenesulphonate or para-roluenesulphonate.

The salts of the compounds of formula (I) also comprise salts with organic or inorganic bases, for example alkali-metal or alkaline-earth metal salts, such as sodium, potassium or calcium salts, sodium and potassium salts being preferred, or with an amine, such as trometamol, or alternatively arginine or lysine salts or salts of any physiologically acceptable amine.

The functional groups optionally present in the molecule of the compounds of formula (I) and in the reaction intermediates can be protected, either in permanent form or in temporary form, with protecting groups which ensure an unequivocal synthesis of the expected compounds.

The expression temporary protecting group for the amines, alcohols or carboxylic acids is understood to mean protecting groups such as those described in “Protective Groups in Organic Synthesis, Greene T. W. and Wuts P. G. M., published John Wiley and Sons, 1991, and in Protecting Groups, Kocienski P. J., 1994, Georg Thieme Verlag.

The compounds (I) can contain precursor groups for other functions which are generated subsequently in one or more other steps.

The compounds of formula (I) in which R, represents a cyclohexyl-(C₁-C₃)alkyl are preferred compounds.

Also preferred are the compounds of formula (I) in which the phenyl in position 5 of the triazole is trisubstituted, preferably with a methoxy in positions 2 and 6 and with a methyl in position 4.

Even more preferred are the compounds of formula (I) in which the phenyl in position 5 of the triazole is trisubstituted, preferably with a methoxy in positions 2 and 5 and with a methyl or a chlorine in position 4.

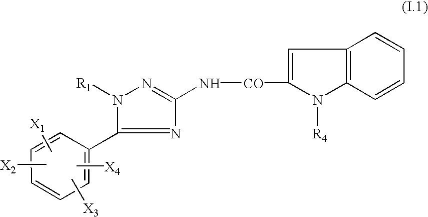

in which R₁, R₄, X₁, X₂, X₃ and X₄ are as defined for (I); a salt or solvate thereof, are preferred.

Among these compounds, those in which

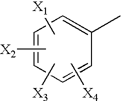

represents 2,6-dimethoxy-4-methylphenyl are preferred.

The compounds of formula:

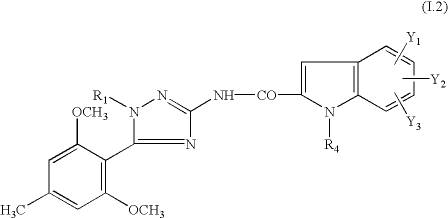

in which R₁ and R₄ are as defined for (I); a salt or solvate thereof, are more particularly preferred.

The compounds of formula:

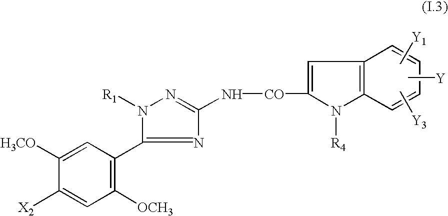

in which R₁, R₄, Y₁, Y₂ and Y₃ are as defined for (1), and X₂ represents methyl or a chlorine atom, a salt or solvate thereof, are most particularly preferred.

The subject of the present invention is also a process for the preparation of the compounds of formula (I), comprising the reaction of an aminotriazole, of formula:

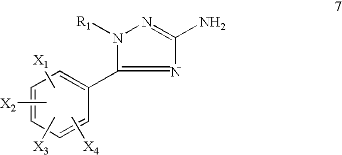

in which R₁, X₁, X₂, X₃ and X₄ are as defined for (I) either with an indolecarboxylic acid derivative of formula:

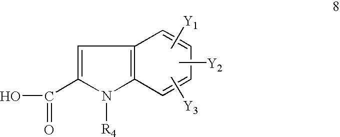

in which R₄, Y₁, Y₂ and Y₃ are as defined above for (1), or with an indolecarboxylic acid derivative of formula:

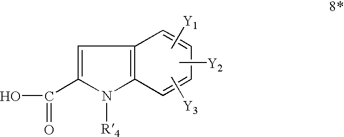

in which Y₁, Y₂ and Y₃ are as defined above for (I) and R′₄ is a precursor group of R₄, in which case the compound of formula:

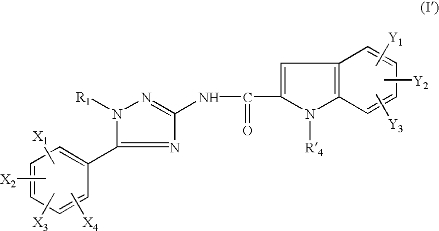

in which R₁, X₁, X₂, X₃, X₄, Y₁, Y₂ and Y₃ are as defined for (I) and R′₄ is a precursor group of R₄, R₄ being as defined for (I);

is formed as an intermediate in order to obtain the compounds of formula (I) or a salt or solvate thereof.

The intermediate compounds (I′) lead to the compounds of formula (I) by conversion of the group R′₄ into R₄, which is carried out in a manner which is known per se according to conventional processes of organic chemistry.

The aminotriazoles of formula 7 constitute novel key intermediates which are useful for the preparation of the compounds (I) and form a subject of the invention.

The starting materials are commercially available or are prepared according to the methods below.

Scheme 1 below illustrates a route for synthesizing the compounds of formula 7.

Scheme 2 below illustrates the preparation of the compounds of formula (I) from the aminotriazoles of formula 7.

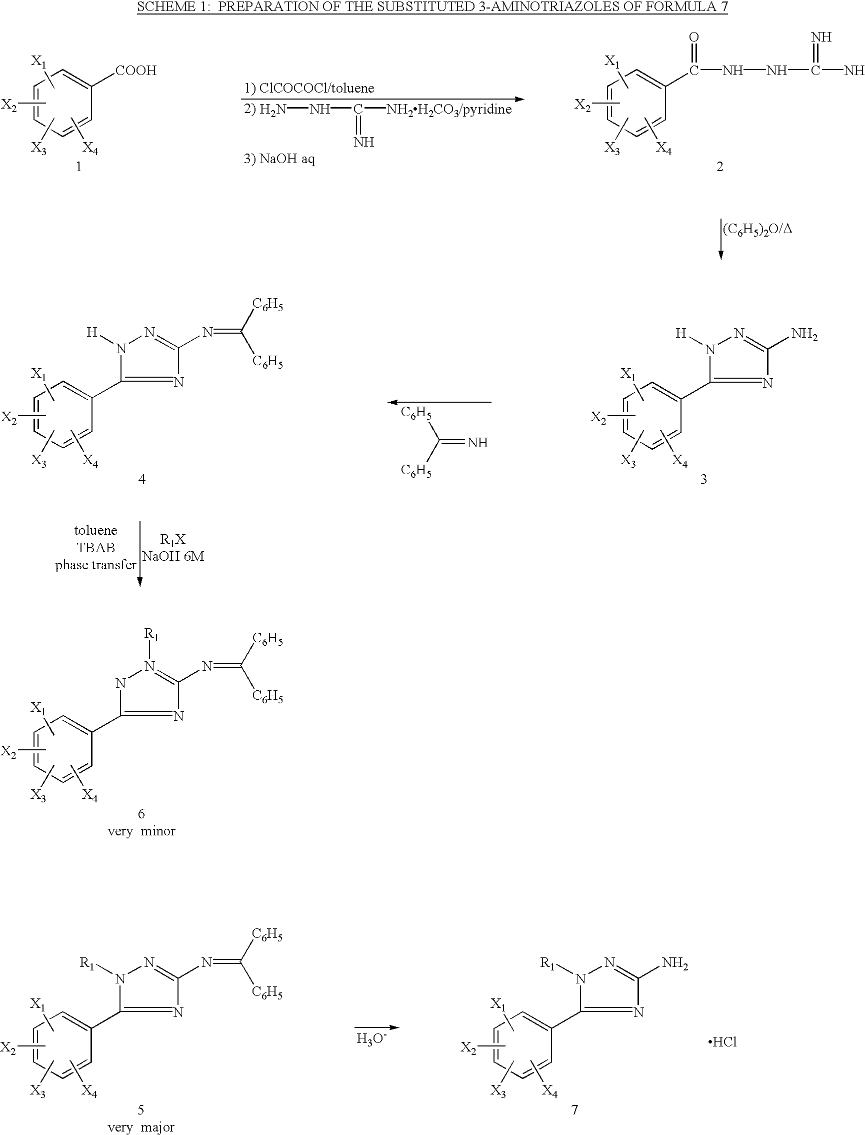

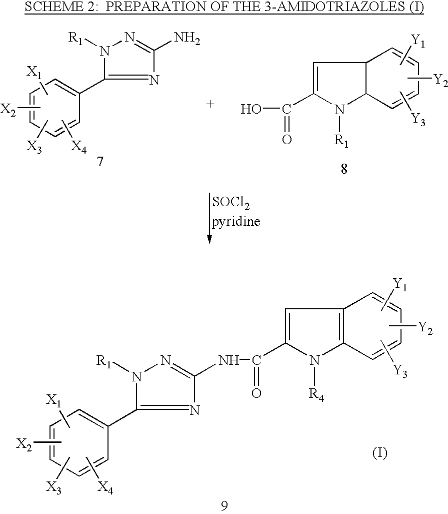

When R₄=—(CH₂)ₙCOOH, the compounds (I) are obtained from the corresponding T esters, which are themselves obtained from Scheme 2.

When R₄=—(CH₂)ₙ-tetrazolyl, the compounds (I) are obtained from the corresponding nitrites of formula:

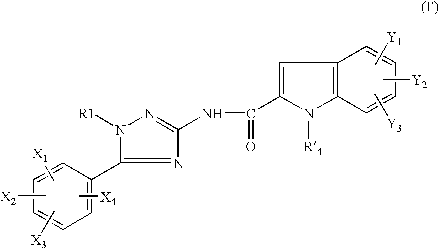

in which R′₄=—(CH₂)ₙ—C≡N

by reacting azidotrimethylsilane in the presence of dibutyltin oxide according to the process described in J. Org. Chem. 1993, 58, 4139-4141.

The compounds of formula (I′) are obtained according to Scheme 2, from compounds 7 and 8′ of formula:

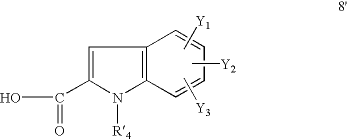

in which R′₄=—(CH₂)ₙ—C≡N.

The substituted benzoic acids are commercially available or are prepared by adaptation of the processes described in the literature, for example:

1) by regioselective, lithiation of substituted benzenes, followed by carboxylation of the lithiated derivative with CO₂, according to Scheme 3:

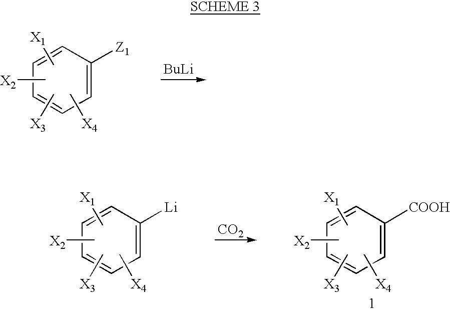

with Z₁=Br or H depending on the nature and/or position of the substituents X₁, X₂, X₃ and X₄, according to N. S. Narasimhan et al., Indian J. Chem., 1973, 11, 1192; R. C. Cambie et al., Austr. J. Chem., 1991, 44, 1465; T. de Paulis et al., J. Med. Chem., 1986, 29, 61; or alternatively

2) by regioselective formylation of substituted benzenes, followed by oxidation of the substituted benzaldehyde with KMnO₄, according to Scheme 4:

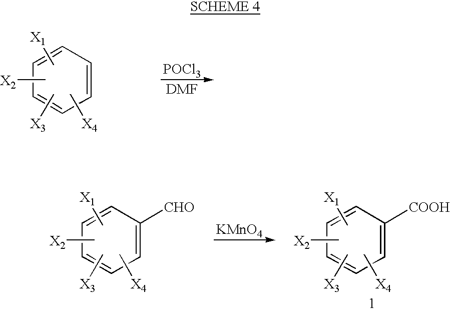

according to the method described by S. B. Matin et al., J. Med. Chem., 1974, 17, 877; or alternatively

3) by haloform oxidation, according to R. Levine et acylation of substituted benzenes (C.A. Bartram et al., J. Chem. Soc., 1963, 4691) or by Fries rearrangement of substituted acyloxybenzenes according to S. E. Cremer et al., J. Org. Chem., 1961, 26, 3653, according to Schemes 5 and 6 below:

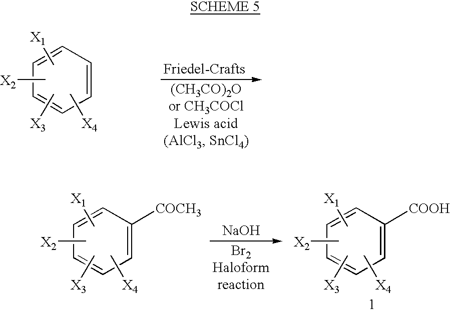

The acids substituted in position 2 with a methoxy can be prepared from a substituted phenol derivative by reaction of acetic anhydride in pyridine, followed by a Fries reaction in the presence of aluminium chloride in order to give the hydroxyacetophenone, on which is reacted methyl iodide in alkaline medium in order finally to obtain, by a haloform reaction, the expected acid 1′ according to Scheme 6 below:

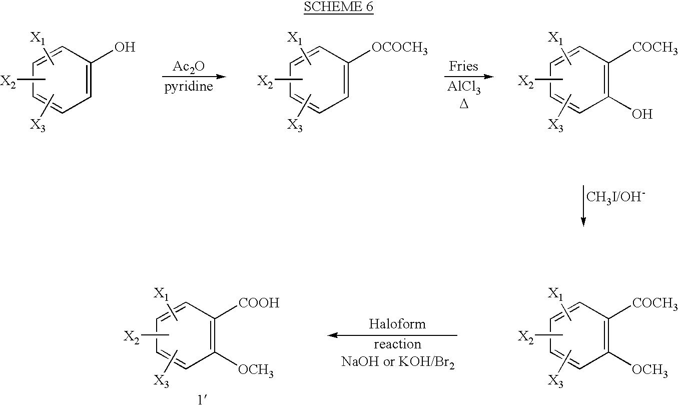

The benzamidoguanidine 2 is obtained by acylation of aminoguanidine hydrogen carbonate with the benzoyl chloride obtained from benzoic acid 1 by standard processes (SOCl₂, oxalyl chloride in an inert solvent), according to an adaptation of the process described by E. Hoggarth, J. Chem. Soc., 1950, 612. It Pan also be obtained according to the alternative route described in this same publication according to Scheme 7 below:

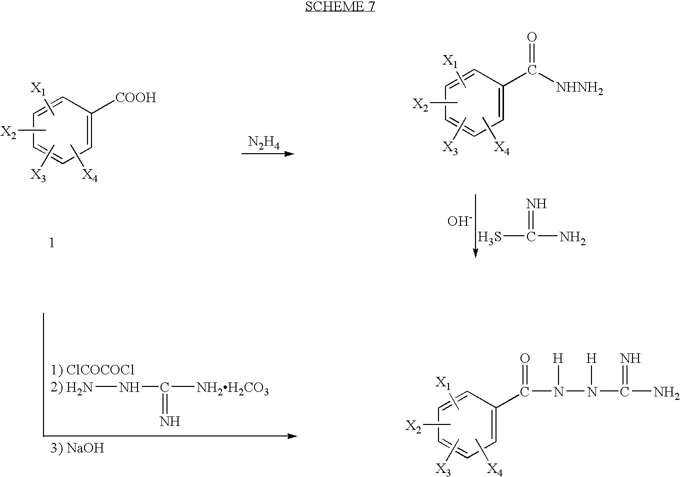

The thermal cyclization of the benzamidoguanidine 2 in a solvent with a high boiling point, such as diphenyl ether, leads to the aryl-5-amino-3-triazole 3 according to an adaptation of the process described by E. Hoggarth, J. Chem. Soc., 1950, 612.

The protection of the primary amino function of the triazole 3 in the form of diphenylimine leads to the N-protected triazole 4, according to an adaptation of a process described by M. J. O'Donnell et al., J. Org. Chem., 1982, 47, 2663.

The compound 4 can also be obtained according to an alternative route which consists in treating the triazole 3, which has been converted beforehand into the hydrochloride 3′, with diphenylimine, according to Scheme 8 below:

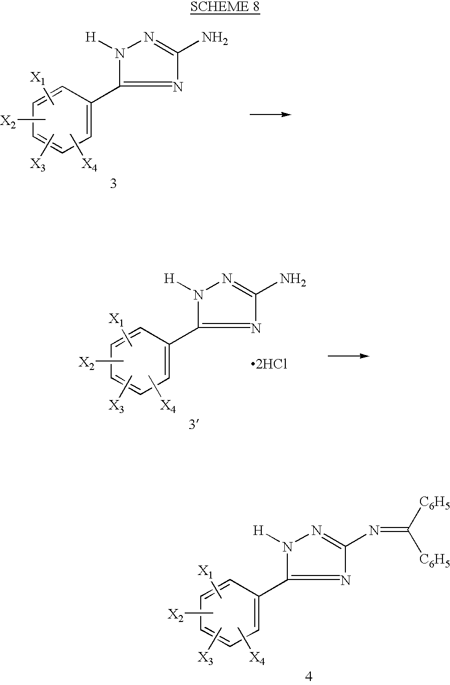

The N-alkylation of the diphenyliminotriazole 4 with an alkyl halide R₁X, under phase transfer conditions (strong base in concentrated aqueous solution, in the presence of an immiscible organic co-solvent and a quaternary ammonium catalyst) leads predominately to the triazole 5, accompanied by a very small amount of the triazole 6. The strong bases used can be aqueous NaOH or KOH solutions at concentrations of 6M to 12M. The cosolvent can be toluene or benzene and the quaternary ammonium can be selected from any quaternary ammonium salt, and more particularly TBAB (tetrabutylammonium bromide).

a) The N-alkylation of the diphenyliminotriazole 4 can be carried out in a non-aqueous medium (dimethylformamide or tetrahydrofuran for example) in the presence of a strong base such as K₂CO₃ or 23 NaH.

b) An alternative route can also be selected, such as the one described by E. Akerblom, Acta Chem. Scand., 1965, 19, 1142, in which an alkylating agent is used in an alcohol such as ethanol in the presence of a solid strong base such as KOH or NaOH.

The triazole 5 is very easily separated from its isomer 6 by chromatography on a column of silica or flash chromatography, depending on the nature of the group R₁. Cleavage of the product 5, obtained after separation from its minor isomer, is carried out in an aqueous acid medium such as 1N HCl, according to an adaptation of the process described by J. Yaozhong et al., Tetrahedron, 1988, 44, 5343 or M. J. O'Donnell et al., d. Org. Chem., 1982, 47, 2663. It allows the amino-3-triazoles N-alkylated in position 1, of formula 7, to be obtained.

The indolcarboxylic compounds of formula 8 were prepared according to processes described in Patent No. EP 611,766 according to Scheme 9 below:

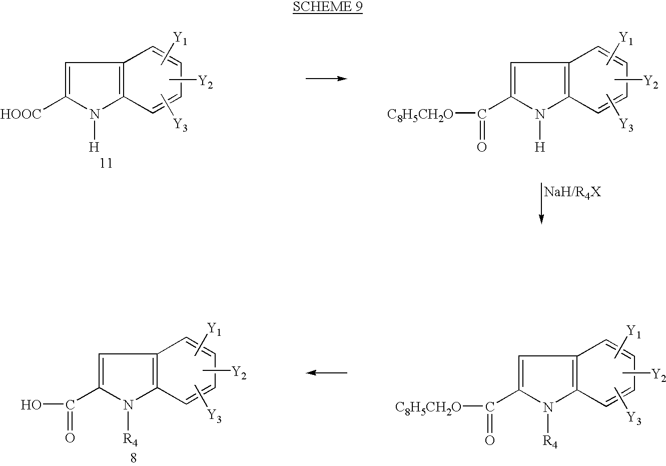

The carboxylic indoles 8 in which R′₄=—(CH₂)ₙ—C≡N

were prepared according to an analogous process presented in Scheme 9a below:

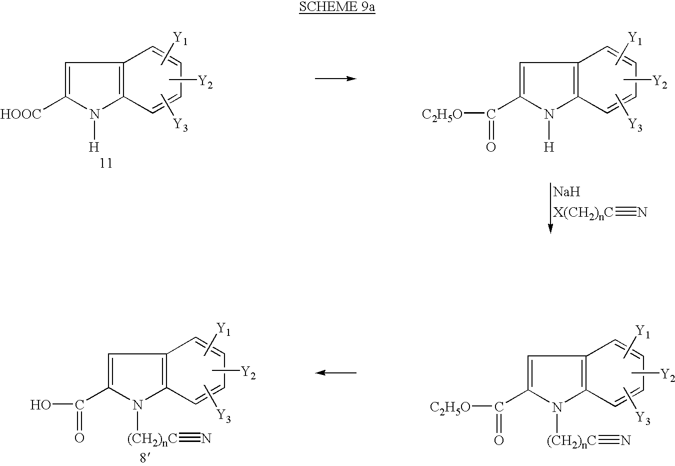

The indoles 11 are commercially available or are prepared by adaptation of the processes described in the literature, for example according to L. Henn et D al., J. Chem. Soc. Perkin Trans. I, 1984, 2189 according to Scheme 10 below:

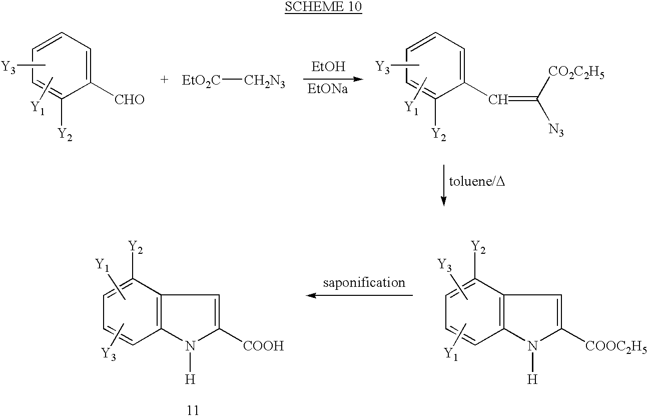

or alternatively, for example, according to the Fischer synthesis (V. Prelog et al., Helv. Chim. Acta., 1948, 31, 1178) according to Scheme 11 below:

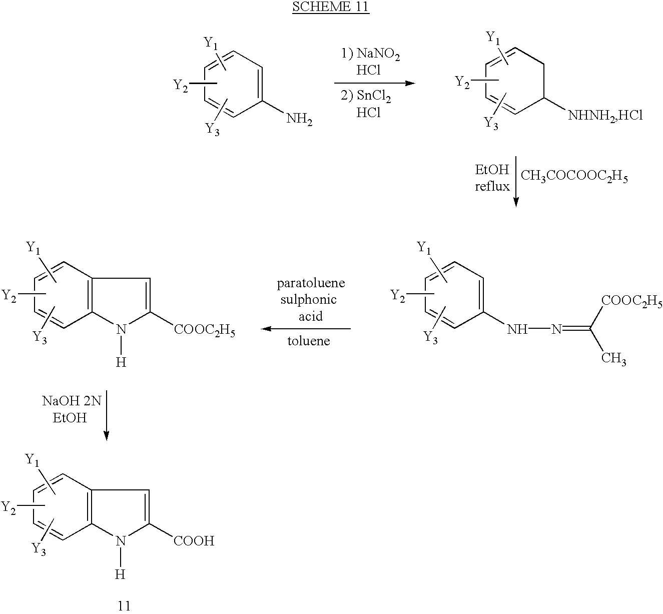

or according to the Japp-Klingemann synthesis (H. Ishii et al., J. Chem. Soc. Perkin. Trans. 1, 1989, 2407) according to Scheme 12 below:

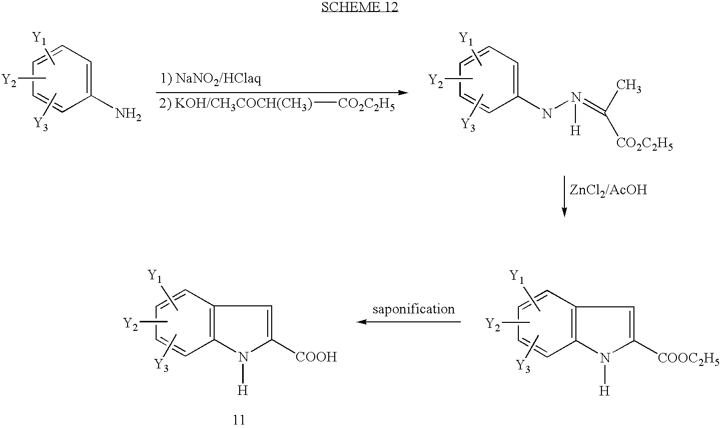

The compounds of formula (I) above also comprise those in which one or more hydrogen, carbon or halogen, in particular chlorine or fluorine atoms have been replaced by their radioactive isotope, for example tritium or carbon-14. Such labelled compounds are useful in research, metabolism or pharmacokinetics studies, in biochemical tests as receptor ligands.

The compounds of formula (I) underwent studies of in vitro binding to the CCK-A and CCK-B receptors, using the method described in Europ. J. Pharmacol. 1993, 232, 13-19.

The agonist activity of the compounds towards the CCK-A receptors was evaluated in vitro in 3T3 cells expressing the human CCK-A receptor, by measuring the mobilization of the intracellular calcium ([Ca⁺⁺]ᵢ), according to a technique derived from that of Lignon M F et al., Eur. J. Pharmacol., 1993, 245, 241-245. The calcium concentration [Ca⁺⁺]ᵢ is evaluated with Fura-2 by the method of the double excitation wavelength. The ratio of the fluorescence emitted at two wavelengths gives the concentration of [Ca⁺⁺]ᵢ after calibration (Grynkiewiez G. et al., J. Biol. Chem., 1985, 260, 3440-3450).

The compounds of the invention stimulate the [Ca⁺⁺]ᵢ partially, or totally such as CCK, and thus behave as CCK-A receptor agonists.

A study of the agonist effect of the compounds on gastric emptying was carried out as follows. Female Swiss albino CD1 mice (20-25 g) are placed on a solid fast for 18 hours. On the day of the experiment, the products (as a suspension in 1% carboxymethyl cellulose solution or in 0.6% methylcellulose solution) or the corresponding vehicle are administered intraperitoneally, 30 minutes before administering a charcoal meal (0.3 ml per mouse of a suspension in water of 10% charcoal powder, 5% gum arabic and 1% carboxymethyl cellulose) or orally one hour earlier. The mice are sacrificed five minutes later by cervical dislocation, and gastric emptying is defined as the presence of charcoal in the intestine beyond the pyloric sphincter (Europ. J. Pharmacol., 1993, 232, 13-19). The compounds of formula (I) partially or completely block gastric emptying, like CCK itself, and thus behave as CCK-receptor agonists. Some of them have ED₅₀ (the effective dose which induces 50% of the effect of CCK) values of less than 0.1 mg/kg intraperitoneally.

A study of the agonist effect of the compounds on gallblader contraction was carried out as follows.

Female Swiss albino CD1 mice (20-25 g) are placed on a solid fast for 24 hours. On the day of the experiment, the products (as a suspension in 1% carboxymethyl cellulose solution or in 0.6% methyl cellulose solution) or the corresponding vehicle are administered orally. The mice are sacrificed by cervical dislocation one hour after administering the products, and the gallbladers are removed and weighed. The results are expressed in mg/kg of body weight (Europ. J. Pharmacol., 1993, 232, 13-19).

The compounds of formula (I) partially or totally contract the gallblader, like CCK itself, and thus behave as CCK-receptor agonists. Some of them have ED₅₀ (the effective dose which induces 50% of the weight decrease of the vesicles observed with CCK) of less than 0.1 mg/kg orally.

Consequently, the compounds of formula (I) are D used as type-A CCK-receptor agonists, for the preparation of medicines intended to combat diseases whose treatment requires stimulation by total or partial agonism of the CCK-A receptors of cholecystokinin. More particularly, the compounds of formula (I) are used for the manufacture of medicines intended for the treatment of certain disorders of the gastrointestinal sphere (prevention of gallstone, irritable bowel syndrome), eating disorders and obesity, and associated pathologies such as diabetes and hypertension. The compounds (I) induce a state of satiety and are thus used to treat eating behaviour disorders, to regulate the appetite and to reduce food intake, to treat bulimia and obesity and to bring about weight loss. The compounds (I) are also useful in emotional and sexual behaviour disorders and memory disorders, in psychosis, and in particular schizophrenia, Parkinson's disease and tardive dyskinesia. They can also serve in the treatment of appetite disorders, i.e. to regulate the desire for eating, in particular the consumption of sugars, carbohydrates, alcohol or drugs and more generally of appetizing ingredients.

The compounds of formula (I) have little toxicity; their toxicity is compatible with their use as medicines for the treatment of the above diseases and disorders.

No signs of toxicity are observed with these compounds at the pharmacologically active doses, and their toxicity is thus compatible with their medical use as medicines.

The subject of the present invention is thus also pharmaceutical compositions containing an effective dose of a compound according to the invention or of a pharmaceutically acceptable salt thereof, and suitable excipients. The said excipients are chosen according to the pharmaceutical composition and the desired mode of administration.

In the pharmaceutical compositions of the present invention for oral, sublingual, subcutaneous, intramuscular, intravenous, topical, intratracheal, intranasal, transdermal, rectal or intraocular administration, the active principles of formula (I) above, or the optional salts thereof, can he administered in unit forms of administration, mixed with standard pharmaceutical supports, to animals and to humans for the prophylaxis or treatment of the above diseases and disorders. The appropriate unit forms of administration comprise oral forms such as tablets, gelatin capsules, powders, granules and oral suspensions and solutions, sublingual, buccal, intratracheal and intranasal forms of administration, subcutaneous, intramuscular or intravenous forms of administration and rectal forms of administration. The compounds according to the invention can be used in creams, ointments, lotions or eye drops for topical administration.

In order to obtain the desired prophylactic or therapeutic effect, the dose of active principle can range between 0.01 and 50 mg per kg of body weight and per day.

Each unit dose can contain from 0.5 to 1000 mg, preferably from 1 to 500 mg, of active ingredients in combination with a pharmaceutical support. This unit dose can be administered 1 to 5 times per day so as to administer a daily dose of from 0.5 to 5000 mg, preferably from 1 to 2500 mg.

When a solid composition in tablet form is prepared, the main active ingredient is mixed with a pharmaceutical vehicle, such as gelatin, starch, lactose, magnesium stearate, talc, gum arabic or the like. The tablets can be coated with sucrose, a cellulose derivative or other suitable materials, or alternatively they can be treated such that they have a sustained or delayed activity and so that they release a predetermined amount of active principle continually.

A preparation in gelatin capsule form is obtained by mixing the active ingredient with a diluent and by pouring the mixture obtained into soft or hard gelatin capsules.

A preparation in syrup or elixir form or for administration in the form of drops can contain the active ingredient together with a sweetener, preferably a calorie-free sweetener, methylparaben and propylparaben as antiseptic, as well as a flavouring agent and a suitable dye. The water-dispersible powders or granules can contain the active ingredient mixed with dispersing agents or wetting agents, or suspension agents such as polyvinylpyrrolidone, as well as with sweeteners or flavour enhancers.

For rectal administration, use is made of suppositories which are prepared with binders that melt at the rectal temperature, for example cocoa butter or polyethylene glycols. Aqueous suspensions, isotonic saline solutions or sterile, injectable solutions which contain pharmacologically compatible dispersing agents and/or wetting agents, for example propylene glycol or butylene glycol, are used for parenteral administration.

The active principle can also be formulated in the form of microcapsules, optionally with one or more supports or additives, or alternatively with matrices such as a polymer or a cyclodextrin (patch, sustained-release forms).

The compositions according to the invention can be used in the treatment or prevention of various complaints in which CCK is of therapeutic value.

The compositions of the present invention can contain, along with the products of formula (I) above or the pharmaceutically acceptable salts thereof, other active principles which can be used in the treatment of the diseases or disorders indicated above.

Advantageously, the compositions of the present invention contain a product of formula (I.1), (I.2) or (I.3) above, or a pharmaceutically acceptable salt, solvate or hydrate thereof.

### Preparation of the Synthetic Intermediates

A. Preparation of the Acids 1 (Variants)

2,5-Dimethoxy-4-methylbenzoic Acid (Compound A.1)

a) 2,5-Dimethoxy-4-methylbenzaldehyde

After stirring a mixture of 8.5 ml of N-methylformanilide (0.068 mol) and 6.3 ml of phosphorus oxytrichloride (0.068 mol) at room temperature for 40 minutes, 17.8 g of 2,5-dimethoxytoluene (0.117 mol) are introduced. The reaction mixture is heated for 6 hours at 50° C. and then, after returning to a temperature of 20° C., it is hydrolysed with 100 ml of aqueous 10% sodium acetate solution, extracted twice with diethyl ether and concentrated. The residue is taken up in aqueous sodium hydrogen sulphite solution and extracted twice with diethyl ether. The aqueous phase is basified (pH=12) in order to give white crystals; m.p.=83° C.; yield=67%.

b) 2,5-Dimethoxy-4-methylbenzoic Acid

23.86 g (0.132 mol) of 2,5-dimethoxy-4-methylbenzaldehyde dissolved in 500 ml of water are heated to 75° C. and 29.3 g (0.185 mol) of potassium permanganate dissolved in 500 ml of water are introduced. The reaction mixture is left for 2 hours at 75° C., the pH is adjusted to 10 with 10% sodium hydroxide solution and the insoluble material is filtered off while hot and rinsed three times with 80 ml of hot water. The filtrate is cooled and the precipitate formed is filtered off and dried under vacuum at 40° C. to give white crystals; m.p.=120° C.; yield=71%.

2,5-Dimethoxy-4-chlorobenzoic Acid (Compound A.2)

a) 2,5-Dimethoxy-4-chlorophenyl Methyl Ketone

162.5 g of aluminium trichloride (1.2 mol) are added, at room temperature, to 2 litres of carbon tetrachloride, followed, at 0° C., by dropwise addition of 82 ml of acetyl chloride (1.2 mol) and then 200 g of 1,4-dimethoxy-2-chlorobenzene (1.2 mol). The reaction mixture is left for 3 and a half hours at 0° C. and is then hydrolysed with 700 ml of water. The organic phase is washed with 2 M sodium hydroxide solution, dried over anhydrous sodium sulphate and concentrated. The semi-crystalline residue is taken up in petroleum ether, filtered and dried to give white crystals; m.p.=96° C.; yield=70%.

b) 2,5-Dimethoxy-4-chlorobenzoic Acid

278 g of potassium hydroxide (4.96 mol) are added to 800 ml of water, followed, at 5° C., by dropwise addition of 84 ml of bromine (1.6 mol). The reaction mixture is left for one hour at room temperature. The aqueous sodium hypobromite solution obtained is added to 107 g of 2,5-dimethoxy-4-chlorophenyl methyl ketone (0.494 mol) dissolved in 1.5 litres of 1,-4-dioxane. After one hour at 20° C., the reaction mixture is heated for one hour at reflux. When the reaction is complete, 100 ml of aqueous sodium hydrogen sulphite solution are introduced and the solvent is then evaporated off. The residue is acidified with 6 N hydrochloric acid solution and is then extracted twice with ethyl acetate. The organic phase is dried over anhydrous sodium sulphate and concentrated. The residue is solidified in diisopropyl ether, to give white crystals; m.p.=160° C.; yield=91%.

2,6-Dimethoxy-4-methylphenylbenzoic Acid (Compound A.3)

231.6 g (1.5 mol) of 3,5-dimethoxytoluene are dissolved in 1 litre of diethylether, followed by dropwise addition, under nitrogen and at room temperature, of 1 litre of a 1.6 N solution of butyllithium (1.6 mol) in hexane. The reaction mixture is left for 18 hours at room temperature and then, after cooling to −30° C., 1 litre of diethyl ether is added and carbon dioxide is bubbled through for one hour, while maintaining the temperature at −30° C. The reaction mixture is taken up in 6 litres of 2 M sodium hydroxide solution, the aqueous phase is separated out after settling has taken place and is acidified with 6 N hydrochloric acid solution. The precipitate formed is filtered off, rinsed with water and dried under vacuum at 40° C. in order to obtain white crystals; m.p.=187° C.; yield=88%.

B. Preparation of Substituted Indoles and Variants Thereof

Preparation of Ethyl 5-methyl-1H-2-indole Carboxylate (Compound B.1)

1st Method: (Japp-Klingemann Method):

7.2 g (0.104 mol) of sodium nitrite dissolved in 40 ml of water are added, at −5° C., to a mixture of 10.7 g (0.1 mol) of 4-methylaniline, 74 ml of 12 N hydrochloric acid and 140 ml of water. The reaction mixture is stirred for 15 minutes at −5° C. and is neutralized by addition of 8.1 g of sodium acetate. 12.33 g (0.085 mol) of ethyl α-methyl-acetoacetate and 80 ml of ethanol are introduced into a three-necked flask, followed, at 0° C., by 4.8 g (0.085 mol) of potassium hydroxide dissolved in 20 ml of water and 100 g of ice. The diazonium solution prepared above is added dropwise, at 0° C., to this reaction mixture and the resulting mixture is left for 18 hours at 0° C. The aqueous phase is extracted 4 times with 50 ml of ethyl acetate and the organic phases are combined and dried over anhydrous sodium sulphate. The residue is taken up in 100 ml of toluene and 16.3 g (0.085 mol) of para-toluene sulphonic acid monohydrate. The mixture is then heated slowly to 110° C. and maintained at this temperature for 5 hours. After cooling and then addition of saturated sodium carbonate solution, the insoluble material is removed by filtration and the organic phase is separated out after settling has taken place, dried over anhydrous sodium sulphate and concentrated. The residue is chromatographed on a column of silica gel, eluent: 30/70 (v/v) dichloromethane/cyclohexane, to give beige-coloured crystals; m.p.=94° C.; yield=25%.

Preparation of Ethyl 4-methyl-1H-2-indolecarboxylate (Compound B2)

2nd Method:

Step 1: Preparation of the Azide

9.3 g (0.405 mol) of sodium are added portionwise to 200 ml of ethanol. 16.2 g (0.135 mol) of ortho-tolualdehyde dissolved in 52.2 g (0.405 mol) of ethyl azidoacetate are introduced dropwise, at −20° C., into this solution of ethoxide in ethanol. After 2 hours at −10° C., the reaction mixture is poured onto 400 ml of water and the precipitate formed is filtered off. It is dried for 18 hours at 40° C. under vacuum in order to obtain white crystals; m.p.=55° C.; yield=78%.

Step 2: Cyclization of the Azide

19.5 g (0.0844 mol) of the azide prepared according to Step 1 are added portionwise to 100 ml of xylene heated to 140° C. Once the addition is complete, the reaction mixture is left for 1 hour at 140° C. The xylene is concentrated and the residue is taken up in isopropyl ether, filtered and dried for 18 hours under vacuum at 40° C., in order to obtain white crystals; m.p.=141° C.; yield=62%.

Preparation of 5-ethyl-1H-2-indolecarboxylic Acid (According to the Fischer Method)—(Compound B.3)

3rd Method:

Step 1: 4-Ethylphenylhydrazine Hydrochloride.

150 ml of water and 160 ml of 12N hydrochloric acid are added to 24.2 g (0.2 mol) of 4-ethylaniline. The mixture is cooled to 0° C. and 14 g (0.2 mol) of sodium nitrite dissolved in 140 ml of water are then introduced dropwise. After 1 hour at 0° C., 112 g (0.496 mol) of stannous chloride dihydrate dissolved in 90 ml of 12 N hydrochloric acid are added to the reaction mixture, at −10° C. After 1 hour 30 at −10° C., the reaction mixture is filtered in order to obtain a brown solid, m.p.=198° C.; yield=95%.

Step 2: Ethyl 2-[2-(4-ethylphenyl)-hydrazono]propanoate

23 ml (0.2 mol) of ethyl pyruvate are added to 34.5 g (0.2 mol) of 4-ethylphenylhydrazine hydrochloride prepared above in suspension in 500 ml of ethanol, and the reaction mixture is heated for 3 hours 30 at reflux. The mixture is then cooled to a temperature of 20° C. and the ethanol is evaporated off. The solid residue is washed with pentane and dried at 40° C. under vacuum in order to obtain a colourless liquid; yield=94%.

Step 3: Ethyl 5-ethyl-1H-2-indolecarboxylate

19 g (0.1 mol) of para-toluene sulphonic acid monohydrate are added portionwise, over 7 hours at reflux, to 44 g (0.188 mol) of hydrazone prepared above, suspended in 300 ml of toluene. The mixture is cooled to a temperature of 20° C. and an insoluble material is separated out by filtration and rinsed with toluene. The filtrate is flashed with saturated aqueous potassium carbonate solution; the phases are separated after settling has taken place and the organic phase is dried over anhydrous sodium sulphate and concentrated. The residue is purified by chromatography on a column of silica gel with the eluent: 5/5 (v/v) dichloromethane/cyclohexane, in order to obtain beige-coloured crystals; m.p.=94° C.; yield=51%.

step 4: 5-Ethyl-1H-2-indolecarboxylic Acid

15.8 g (0.073 mol) of ethyl 5-ethyl-2-indolecarboxylate prepared according to Step 3 are added to 150 ml of 1,4-dioxane, followed by 45 ml of 2 M sodium hydroxide solution (0.09 mol). The reaction mixture is left for 48 hours at room temperature. After evaporation of the 1,4-dioxane, the residue is taken up in 6 N hydrochloric acid solution and the precipitate formed is filtered off and dried under vacuum at 60° C. in order to give the 5-ethyl-1H-2-indolecarboxylic acid in the form of white crystals; m.p.=184° C.; yield=92%.

Preparation of the N-Alkyl 1H-2-Indolecarboxylic Acids

5-Ethyl-1-(methoxycarbonylmethyl)-1H-2-indolecarboxylic Acid—(Compound B.4)

Step 1: Benzyl 5-ethyl-1H-2-indolecarboxylate

12.7 g (0.067 mol) of 5-ethyl-1H-2-indolecarboxylic acid and 10 ml of 1,8-diazabicyclo[5.4.0]undec-7-ene (0.067 mol) are successively added to 70 ml of dimethylformamide. The reaction mixture is left for 40 minutes at 0° C., after which 10.6 ml of benzyl bromide (0.089 mol) are introduced dropwise. After reaction for 18 hours at room temperature, the reaction mixture is poured onto 300 ml of water and the precipitate formed is filtered off, rinsed with water and then dried for 18 hours at 50° C. under vacuum in order to give yellow crystals: m.p.=99° C.; yield=90%.

Step 2: Benzyl 5-ethyl-1-(methoxycarbonylmethyl)-1H-2-indolecarboxylate

75 ml of dimethylformamide are added to 1.5 g (0.031 mol) of sodium hydride as a 50° suspension in oil, followed by portionwise addition of 7.9 g (0.0283 mol) of benzyl 5-ethyl-1H-2-indolecarboxylate prepared according to Step 1. After 40 minutes at 0° C., 3.5 ml (0.0315 mol) of methyl bromoacetate are introduced dropwise and the reaction mixture is left for 2 hours at 20° C. 300 ml of ethyl acetate are added, the mixture is washed with 2×300 ml of water, the phases are then separated after settling has taken place and the organic phase is dried over anhydrous sodium sulphate and concentrated. 9.5 g of colourless oil are obtained; yield=95%.

Step 3: 5-Ethyl-1-(methoxycarbonylmethyl)-1H-2-indolecarboxylic Acid

2.5 g of 10% Pd/C are added to 9.5 g (0.0269 mol) of benzyl 5-ethyl-1-(methoxycarbonylmethyl)-1H-2-indolecarboxylate prepared according to Step 2, dissolved in 150 ml of ethanol, followed by addition of 40 ml of cyclohexene (0.395 mol). The reaction mixture is heated for 2 hours at 70° C. and is then cooled to a temperature of 20° C. The reaction mixture is filtered through talc and the filtrate is evaporated to dryness. The residue is dried for 18 hours at 40° C. under vacuum, in order to give beige-coloured crystals; m.p.=181° C.; yield=90%.

Compounds B5 to B70 described in Table I below are synthesized by working according to the above Preparations, starting with appropriate synthetic intermediates.

TABLE I

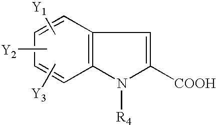

| COMPOUND No. | Y₁ | Y₂ | Y₃ | R₄ | m.p.: ° C. |
|---|---|---|---|---|---|
| B5 | 5-C₂H₅ | H | H | —(CH₂)₂CO₂CH₃ | 128 |
| B6 | 5-C₂H₅ | H | H | —(CH₂)₃CO₂C₂H₅ | 94 |
| B7 | 5-C₂H₅ | H | H | —(CH₂)₄CO₂C₂H₅ | oil |
| B8 | 4-CH₃ | 5-CH₃ | H | —(CH₂CO₂CH₃ | 208 |
| B9 | 4-CH₃ | 5-CH₃ | H | —(CH₂)₂CO₂CH₃ | 170 |
| B10 | 4-CH₃ | 5-CH₃ | H | —(CH₂)₃CO₂C₂H₅ | 183 |
| B11 | 5-C₂H₅ | H | H | —(CH₂)₃CO₂C₂H₅ | oil |
| B12 | 5-Cl | H | H | —CH₂CO₂CH₃ | 207 |
| B13 | 5-Cl | H | H | —(CH₂)₂CO₂CH₃ | 175 |
| B14 | 5-Cl | H | H | —(CH₂)₃CO₂C₂H₅ | 152 |
| B15 | 5-Cl | H | H | —(CH₂)₄CO₂C₂H₅ | 99 |
| B16 | 5-Cl | H | H | —(CH₂)₃CO₂C₂H₅ | 93 |
| B17 | 5-CH₃ | H | H | —CH₂CO₂CH₃ | 211 |
| B18 | 5-CH₃ | H | H | —(CH₂)₂CO₂CH₃ | 174 |
| B19 | 5-CH₃ | H | H | —(CH₂)₄CO₂C₂H₅ | 188 |
| B21 | 5-CH₃ | H | H | —(CH₂)₃CO₂C₂H₅ | 91 |
| B22 | 4-OCH₃ | 5-CH₃ | 6-OCH₃ | —CH₂CO₂CH₃ | 220 |
| B23 | 4-OCH₃ | 5-CH₃ | 6-OCH₃ | —CH₂CH₂CO₂CH₃ | 200 |
| B24 | 4-OCH₃ | 5-CH₃ | 6-OCH₃ | —(CH₂)₃CO₂C₂H₅ | 134 |
| B25 | 5-OCH₃ | H | H | —CH₂CO₂CH₃ | 195 |
| B26 | 5-OCH₃ | H | H | —(CH₂)₂CO₂CH₃ | 157 |
| B27 | 5-OCH₃ | H | H | —(CH₂)₃CO₂C₂H₅ | 119 |
| B28 | 5-OCH₃ | H | H | —(CH₂)₄CO₂C₂H₅ | 87 |
| B29 | 5-OCH₃ | H | H | —(CH₂)₃CO₂C₂H₅ | 70 |
| B30 | 5-CH₃ | H | H | —CH₃ | 230 |
| B31 | 5-CH₃ | H | H | —CH₂CH₃ | 206 |
| B32 | 5-CH₃ | H | H | —CH₂CH₂OCH₃ | 158 |
| B33 | 5-OCH₃ | H | H | —CH₂CH₂OCH₃ | 170 |
| B34 | 4-CH₃ | H | H | —CH₂CO₂CH₃ | 206 |
| B35 | 4-CH₃ | H | H | —(CH₂)₂CO₂CH₃ | 118 |
| B36 | 5-OC₂H₅ | H | H | —CH₂CO₂CH₃ | 188 |
| B37 | 5-OC₂H₅ | H | H | —(CH₂)₂CO₂CH₃ | 158 |
| B38 | 5-OC₂H₅ | H | H | —(CH₂)₃CO₂C₂H₅ | 131 |
| B39 | 4-OCH₃ | 6-OCH₃ | H | —CH₂COOCH₃ | 195 |
| B40 | 4-OCH₃ | 6-OCH₃ | H | —(CH₂)₂COOCH₃ | 191 |
| B41 | 4-OCH₃ | 6-OCH₃ | H | —(CH₂)₃COOC₂H₅ | 154 |
| B42 | 4-OCH₃ | 5-CH₃ | 6-OCH₃ | —(CH₂)₃CO₂C₂H₅ | 132 |
| B43 | 5-Cl | H | H | —CH₃ | 248 |
| B44 | 5-CH₃ | H | 7-CH₃ | —CH₂CO₂CH₃ | 208 |
| B45 | 5-CH₃ | H | 7-CH₃ | —(CH₂)₂CO₂CH₃ | — |
| B46 | 5-CH₃ | H | 7-CH₃ | —(CH₂)₃CO₂C₂H₅ | 183 |
| B47 | 5-Cl | H | H | —(CH₂)₂OCH₃ | 182 |
| B48 | 4-CH₃ | 5-CH₃ | 6-OCH₃ | —CH₂CO₂CH₃ | 185 |
| B49 | 4-CH₃ | 5-CH₃ | 6-OCH₃ | —CH₂CH₂CO₂CH₃ | 197 |
| B50 | 4-CH₃ | 5-CH₃ | 6-OCH₃ | —(CH₂)₃COOC₂H₅ | 143 |
| B51 | 4-CH₃ | H | 7-CH₃ | —CH₂COOCH₃ | 118 |
| B52 | 4-CH₃ | H | 7-CH₃ | —(CH₂)₃COOC₂H₅ | 108 |
| B53 | 5-OCH₃ | H | 7-CH₃ | —CH₂COOCH₃ | 215 |
| B54 | 4-CH₃ | 6-CH₃ | H | —CH₂COOCH₃ | 112 |
| B55 | 4-CH₃ | 6-CH₃ | H | —(CH₂)₂COOC₂H₅ | 152 |
| B58 | 6-C₂H₆ | H | H | —CH₂COOCH₃ | 158 |
| B57 | 6-C₂H₆ | H | H | —(CH₂)₃COOC₂H₅ | 142 |
| B58 | 5-OCH₃ | H | 7-CH₃ | —(CH₂)₃COOC₂H₅ | oil |
| B59 | 6-C₂H₅ | H | H | —(CH₂)₂COOCH₃ | 166 |
| B60 | 5-Cl | H | 7-CH₃ | —CH₂COOCH₃ | 209 |
| B61 | 5-OCH₃ | H | 7-OCH₃ | —CH₂COOHCH₃ | 186 |
| B62 | 5-OCH₃ | H | 7-OCH₃ | —(CH₂)₃COOC₂H₅ | 138 |
| B63 | 5-OCH₃ | 6-OCH₃ | H | —CH₂COOCH₃ | 202 |
| B64 | 5-F | H | 7-CH₃ | —CH₂COOCH₃ | 242 |
| B65 | 5-F | H | 7-CH₃ | —(CH₂)₃COOC₂H₅ | 142 |
| B66 | 5-Cl | H | 7-CH₃ | —(CH₂)₃COOC₂H₅ | 181 |
| B67 | 5-OCH₃ | 6-OCH₃ | H | —(CH₂)₂COOCH₃ | 166 |
| B68 | 5-OCH₃ | 6-OCH₃ | H | —(CH₂)₃COOC₂H₅ | oil |
| B69 | 5-CH₃ | 7-Cl | H | —CH₂COOCH₃ | 210 |
| B70 | 4-CH₃ | 6-OCH₃ | 7-CH₃ | —CH₂COOCH₃ | 211 |

4,5-Dimethyl-1-(3-cyanopropyl)-1H-2-indolecarboxylic acid (Compound B71)

Step 1: Ethyl 4,5-dimethyl-1-(3-cyanopropyl)-1H-2-indolecarboxylate

75 ml of dimethylformamide are added to 1.92 g (0.040 mol) of sodium hydride as a 50% suspension in oil, followed by portionwise addition of 7.9 g (0.0363 mol) of ethyl 4,5-dimethyl-1H-2-indole-carboxylate. After stirring for 40 minutes at 0° C., 4.0 ml (0.040 mol) of 4-bromobutyronitrile are introduced dropwise and the reaction mixture is maintained for 2 hours at 20° C. 300 ml of ethyl acetate are added, the mixture is washed with twice 300 ml of water, the phases are separated after settling has taken place and the organic phase is then dried over anhydrous sodium sulphate and concentrated. 9.8 g of colourless oil are obtained; Yield=95%.

Step 2: 4,5-Dimethyl-1-(3-cyanopropyl)-1H-2-indole-carboxylic Acid

9.8 g (0.0345 mol) of ethyl 4,5-dimethyl-1-(3-cyanopropyl)-1H-2-indolecarboxylate are added to 150 ml of 1,4-dioxane, followed by addition of 25 ml of 2 M sodium hydroxide solution (0.05 mol). The reaction mixture is maintained for 48 hours at room temperature. After evaporation of the 1,4-dioxane, the residue is taken up in 6 M hydrochloric acid solution and the precipitate formed is filtered off and dried under reduced pressure at 60° C. in order to give the 4,5-dimethyl-1-(3-cyanopropyl)-1H-2-indole-carboxylic acid in the form of white crystals; m.p.=175° C., yield=92%.

Compounds B72 to B75 presented in Table Ia below are prepared in the same way.

TABLE Ia

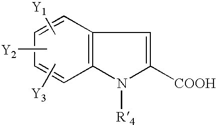

| COMPOUND | | | | | m.p.: |
|---|---|---|---|---|---|
| No. | Y₁ | Y₂ | Y₃ | R′₄ | ° C. |
| B72 | 5-C₃H₅ | H | H | —(CH₂)₃—C≡N | 137 |
| B73 | 5-C₂H₅ | H | H | —CH₂—C≡N | 229 |
| B74 | 5-OCH₃ | H | H | —CH₂—C≡N | 190 |
| B75 | 5-CH₃ | 6-CH₃ | 7-OCH₃ | —(CH₂)₃—C≡N | 181 |

C. Preparation of the Benzamidoguanidine Derivatives Preparation of 2,6-dimethoxy-4-methylbenzamidoguanidine (Compound C.1)

1 ml of dimethylformamide is added to 353 g (1.8 mol) of 2,6-dimethoxy-4-methylbenzoic acid suspended in 1.5 litres of toluene, followed by dropwise addition of 190 ml of oxalyl chloride (2.16 mol). The reaction mixture is left for two hours at room temperature and is then evaporated to dryness. The crystalline residue is added portion-wise to a suspension of 293.8 g of aminoguanidine hydrogen carbonate (2.16 mol) in 2.5 litres of pyridine at ±5° C. and is left for 18 hours at 20° C. The reaction mixture is evaporated to dryness and the residue is then taken up in 1 litre of 2 M sodium hydroxide solution. The precipitate is filtered off and is rinsed with a minimum amount of water and then dried under vacuum at 60° C. in order to obtain a crystalline residue; m.p.=222° C.; yield=81%.

D. Preparation of the 3-Aminotriazole Derivatives

3-Amino-5-(2,6-dimethoxy-4-methylphenyl)-1,2,4-triazole (Compound D.1)

2 litres of diphenyl ether are added to 230 g (0.91 mol) of 2,6-dimethoxy-4-methylbenzamidoguanidine, after which the reaction mixture is heated for 5 minutes at 220° C. The mixture is cooled to 80° C. and the precipitate is then filtered off, rinsed with diisopropyl ether and dried under vacuum at 60° C. in order to obtain crystals; m.p.=286° C.; yield=93%.

Compound D2 to D11 described in Table II below are synthesized in the same way, by working according to this Preparation and using the appropriate starting materials.

TABLE II

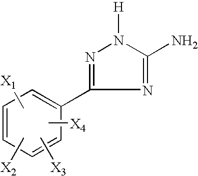

| COMPOUND | | | | | m.p.: |
|---|---|---|---|---|---|
| No. | X₁ | X₂ | X₃ | X₄ | ° C. |
| D2 | 2-OCH₃ | 4-OCH₃ | 6-OCH₃ | H | 297 |
| D3 | 2-OCH₃ | 4-OCH₃ | 5-OCH₃ | H | 240 |
| D4 | 2-OCH₃ | 4-CH₃ | 5-OCH₃ | H | 248 |
| D5 | 2-OCH₃ | 4-Cl | 5-OCH₃ | H | 282 |
| D6 | 2-OCH₃ | 4-CH₃ | 6-CH₃ | H | 286 |
| D7 | 2-OCH₃ | 4-OCH₃ | 5-CH₃ | H | 248 |
| D8 | 2-OCH₃ | 4-CH₃ | 5-CH₃ | H | 286 |
| D9 | 2-OCH₃ | 3-Cl | 6-OCH₃ | H | 215 |
| D10 | 2-OCH₃ | 3-CH₃ | 6-OCH₃ | H | 236 |
| D11 | 2-OCH₃ | 4-CH₃ | 5-CH₃ | 6-OCH₃ | 237 |

E. Preparation of the Diphenylimino Derivatives

Preparation of N-[3-(2,6-dimethoxy-4-methylphenyl)-1H-1,2,4-triazol-5-yl]-N-diphenylmethyleneamine (Compound E.1)

105 g (0.45 mol) of 3-amino-5-(2,6-dimethoxy-4-methylphenyl)-1,2,4-triazole suspended in 200 ml of xylene and 150 g (0.9 mol) of benzophenoneimine are heated at 140° C. for 48 hours under a stream of argon. The reaction mixture is cooled to a temperature of 80° C. and is then poured into 4 litres of isopropyl ether and the precipitate formed is filtered off, rinsed with diisopropyl ether and dried for 18 hours at 50° C.; m.p.=126° C.; yield 90%.

TABLE III

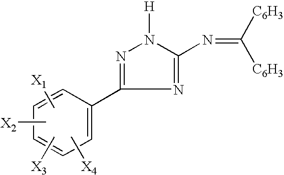

| COMPOUND No. | X₁ | X₂ | X₃ | X₄ | m.p.:° C. |
|---|---|---|---|---|---|
| E2 | 2-OCH₃ | 4-OCH₃ | 6-OCH₃ | H | 143 |
| E3 | 2-OCH₃ | 4-OCH₃ | 5-OCH₃ | H | 235 |
| E4 | 2-OCH₃ | 4-CH₃ | 5-OCH₃ | H | 228 |
| E5 | 2-OCH₃ | 4-Cl | 5-OCH₃ | H | 236 |
| E6 | 2-OCH₃ | 4-CH₃ | 5-CH₃ | H | 171 |
| E7 | 2-OCH₃ | 4-CH₃ | 5-CH₃ | H | 240 |
| E8 | 2-OCH₃ | 3-Cl | 6-OCH₃ | H | 152 |
| E9 | 2-OCH₃ | 3-CH₃ | 6-OCH₃ | H | 169 |
| E10 | 2-OCH₃ | 4-CH₃ | 5-CH₃ | 6-OCH₃ | 110 |

F. Preparation of the 1-Substituted 3-Amino Triazoles

Preparation of 1-(2-cyclohexylethyl)-5-(2,6-dimethoxy-4-methylphenyl)-1H-1,2,4-triazol-3-amine (Compound F.1)

a) N-Alkylation of the Triazole

300 ml of aqueous 6 N sodium hydroxide solution, 24 g (0.06 mol) of N-[3-(2,6-dimethoxy-4-methylphenyl)-1H-1,2,4-triazol-5-yl]-N-diphenylmethyleneamine and 2.7 g of tetrabutylammonium bromide are added successively to 400 ml of toluene. 17 g (0.09 mol) of 2-bromoethyl cyclohexane are added dropwise to the reaction mixture, heated to 70° C. The reaction is continued for two hours at 80° C. The organic phase is separated out after settling has taken place and is dried over anhydrous sodium sulphate and evaporated to dryness. The residue is chromatographed on a column of silica gel with the eluent: 90/10 (v/v) toluene/ethyl acetate. 21.4 g of colourless oil are obtained; yield=70%.

b) Hydrolysis of the Diphenylimine Function

100 ml of 1N hydrochloric acid solution are added to 10.3 g (0.02 mol) of N-[1-(2-cyclohexylethyl)-5-(2,6-dimethoxy-4-methylphenyl)-1H-1,2,4-triazol-3-yl]-N-diphenylmethyleneamine dissolved in 200 ml of methanol. The reaction mixture is left for 18 hours at room temperature and is then evaporated to dryness. The oily residue is solidified in diethyl ether and the precipitate obtained is filtered off and dried under vacuum at 40° C.; m.p.=136° C. (hydrochloride); yield=90%.

TABLE IV

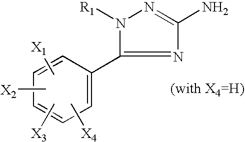

| | | | | | m.p.: ° C. |
|---|---|---|---|---|---|
| COMPOUND No. | X₁ | X₂ | X₄ | R₁ | (hydrochloride) |
| F2 | 2-OCH₃ | 4-CH₃ | 6-OCH₃ | 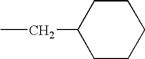 | 135 |
| F3 | 2-OCH₃ | 4-CH₃ | 6-OCH₃ | —CH₂—C₆H₅ | 215 |
| F4 | 2-OCH₃ | 4-CH₃ | 6-OCH₃ | —(CH₂)₄—CH₃ | 143 |
| F5 | 2-OCH₃ | 4-CH₃ | 6-OCH₃ | 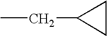 | 238 |
| F6 | 2-OCH₃ | 4-CH₃ | 6-OCH₃ | —CH₂CH₂—CH₆H₅ | 200 |
| F7 | 2-OCH₃ | 4-CH₃ | 6-OCH₃ | —(CH₂)₄—CH—(CH₂)₂ | 172 |
| F8 | 2-OCH₃ | 4-CH₃ | 6-OCH₃ | 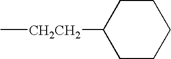 | 187 |
| F9 | 2-OCH₃ | 4-CH₃ | 6-OCH₃ | 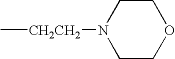 | 180 |
| F10 | 2-OCH₃ | 4-CH₃ | 6-OCH₃ | —(CH₂)₂—N(CH₂)₂ | 148 |
| F11 | 2-OCH₃ | 4-CH₃ | 6-OCH₃ | 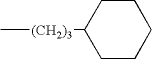 | 190 |
| F12 | 2-OCH₃ | 4-CH₃ | 6-OCH₃ | —(CH₂)₃—CH₃ | 212 |
| F13 | 2-OCH₃ | 4-CH₃ | 6-OCH₃ | 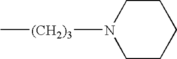 | 198 |
| F14 | 2-OCH₃ | 4-CH₃ | 6-OCH₃ | 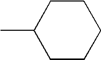 | 219 |
| F15 | 2-OCH₃ | 4-CH₃ | 6-OCH₃ | —CH₂—CH—(C₆H₅)₂ | 132 |
| F16 | 2-OCH₃ | 4-CH₃ | 6-OCH₃ | 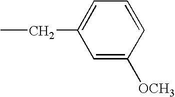 | 197 |
| F17 | 2-OCH₃ | 4-CH₃ | 6-OCH₃ | 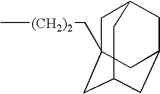 | 217 |
| F18 | 2-OCH₃ | 4-CH₃ | 6-OCH₃ | 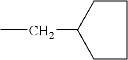 | 208 |
| F19 | 2-OCH₃ | 4-CH₃ | 6-OCH₃ | 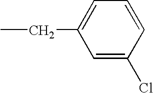 | 136 |
| F20 | 2-OCH₃ | 4-CH₃ | 6-OCH₃ | 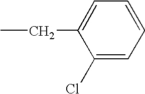 | 204 |
| F21 | 2-OCH₃ | 4-CH₃ | 6-OCH₃ | 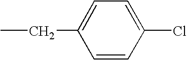 | 202 |
| F22 | 2-OCH₃ | 4-CH₃ | 6-OCH₃ | 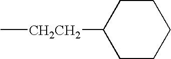 | 196 |
| F23 | 2-OCH₃ | 4-CH₃ | 5-OCH₃ | 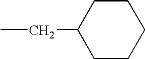 | 148 |
| F24 | 2-OCH₃ | 4-CH₃ | 5-OCH₃ | 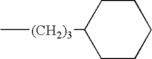 | 192 |
| F25 | 2-OCH₃ | 4-CH₃ | 5-OCH₃ | 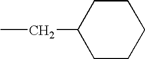 | 188 |
| F26 | 2-OCH₃ | 4-CH₃ | 5-OCH₃ | 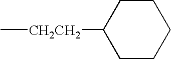 | 168 |
| F27 | 2-OCH₃ | 4-CH₃ | 6-OCH₃ | 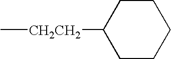 | 189 |
| F28 | 2-OCH₃ | 4-CH₃ | 6-OCH₃ | 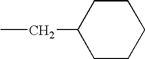 | 180 |
| F29 | 2-OCH₃ | 4-CH₃ | 6-OCH₃ | 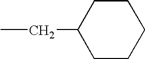 | 168 |
| F30 | 2-OCH₃ | 4-CH₃ | 6-OCH₃ | 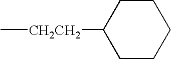 | 188 |
| F31 | 2-OCH₃ | 4-CH₃ | 5-OCH₃ | 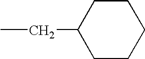 | 200 |
| F32 | 2-OCH₃ | 4-CH₃ | 5-OCH₃ | 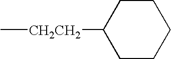 | 206 |
| F33 | 2-OCH₃ | 4-CH₃ | 6-OCH₃ | —CH₂CH₂CN | 244 |
| F34 | 2-OCH₃ | 4-CH₃ | 5-OCH₃ | 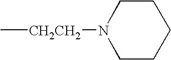 | 218 |
| F35 | 2-OCH₃ | 4-Cl | 5-OCH₃ | 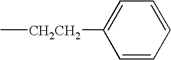 | 127 |
| F36 | 2-OCH₃ | 4-Cl | 6-OCH₃ | 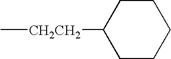 | 159 |
| F37 | 2-OCH₃ | 3-CH₃ | 6-OCH₃ | 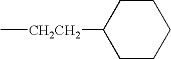 | 168 |

1-(2-Cyclohexylethyl)-5-(2,6-dimethoxy-4,5-di-methylphenyl)-1H-1,2,4-triazol-3-amine (Compound F38) is prepared in a similar manner, starting with Compound E10; m.p.=180° C.

G. Preparation of the Amidotriazole Derivatives with Non-N-Substituted Indoles

Synthesis of N-[1-(2-chlorobenzyl)-5-(2,6-dimethoxy-4-methylphenyl)-1H-1,2,4-triazol-3-yl]-5-chloro-1H-2-indolecarboxamide (Compound G.1).

0.2 ml of thionyl chloride (0.0028 mol) is added, at 0° C., to a solution of 1 ml of pyridine (0.013 mol) in 30 ml of methylene chloride. After 15 minutes at 0° C., 500 mg (0.0025 mol) of 5-chloroindolecarboxylic acid are introduced and the reaction mixture is left for 30 minutes at 0° C. 0.91 g (0.0028 mol) of 1-[(2-chlorophenyl)methyl]-5-(2,6-dimethoxy-4-methylphenyl)-1H-1,2,4-triazole-3-amine hydrochloride is added to the acyl chloride formed and the mixture is left for 18 hours at 20° C.

The reaction mixture is washed with 1 M sodium hydroxide solution. The organic phase is dried over anhydrous sodium sulphate and evaporated to dryness. The residue is chromatographed on silica gel with the eluent: 95/5 (v/v) dichloromethane/methanol, to give 0.980 g of crystals: m.p.=262° C.; yield=73%.

TABLE V

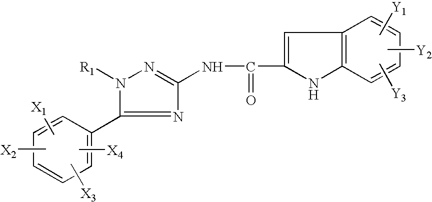

| COMPOUND No. | 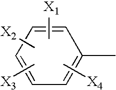 | R₁ | 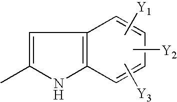 | m.p.:° C. |
|---|---|---|---|---|
| G2 | 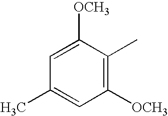 | 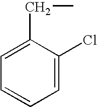 | 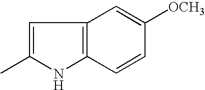 | 271 |
| G3 | 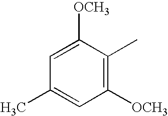 | 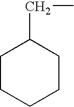 | 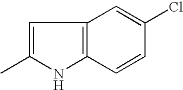 | 301 |
| G4 | 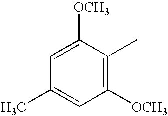 | 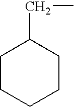 | 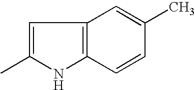 | 251 |
| G5 | 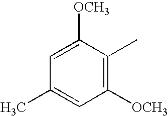 | 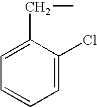 | 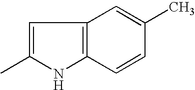 | 248 |
| G8 | 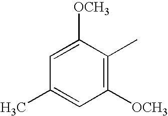 | 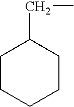 | 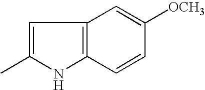 | 283 |
| G7 | 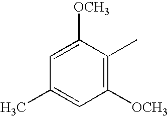 | 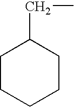 | 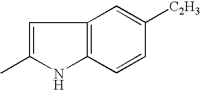 | 253 |
| G8 | 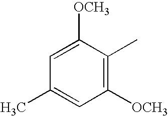 | 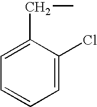 | 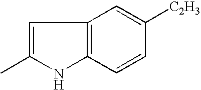 | 229 |
| G9 | 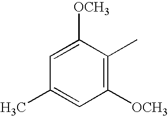 | 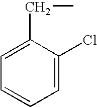 | 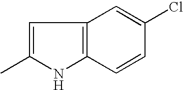 | 262 |
| G10 | 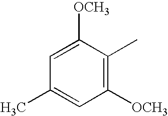 | 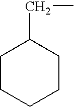 | 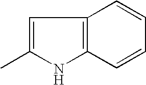 | 270 |
| G11 | 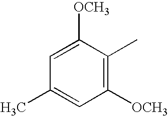 | 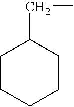 | 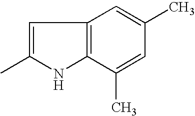 | 245 |
| G12 | 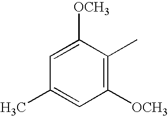 | 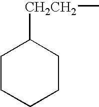 | 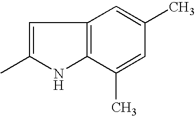 | 139 |
| G13 | 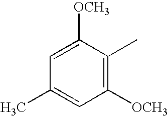 | 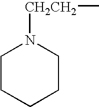 | 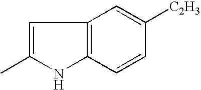 | 210 (HCl) |
| G14 | 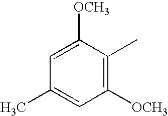 | 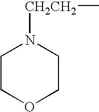 | 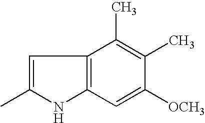 | 210 (HCl) |
| G15 | 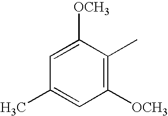 | 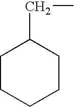 | 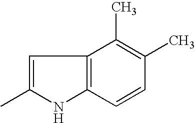 | 252 |
| G18 | 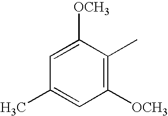 | 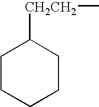 | 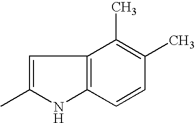 | 181 |

H. Preparation of the Aminotriazole Derivatives with N-Substituted Indoles

### EXAMPLE 1

Methyl 2-[2-({[1-(2-cyclohexylethyl)-5-(2,6-dimethoxy-4-methylphenyl)-1H-1,2,4-triazol-3-yl]amino}carbonyl)-5-ethyl-1H-indol-1-yl]acetate

1 ml of pyridine (0.013 mol) and 0.21 ml of thionyl chloride (0.00029 mol) are added successively to 15 ml of dichloromethane. After 15 minutes at 0° C., 0-0.627 g of 5-ethyl-1-methoxycarbonylmethyl-1H-2-indolecarboxylic acid (0.0024 mol) is introduced, followed by 0.9 g of 1-(2-cyclohexylethyl)-5-(2,6-dimethoxy-4-methylphenyl)-1H-1,2,4-triazole-3-amine hydrochloride. The reaction mixture is left for 18 hours at room temperature, after which an acidic washing and then a basic washing are carried out. The organic phase is dried over anhydrous sodium sulphate and concentrated. The oily residue is chromatographed on silica gel with the eluent: 98.5/1.5 (v/v) dichloromethane/methanol, to give a white powder; m.p.=191° C.; yield=87%.

### EXAMPLE 2

2-[2-({[1-(2-Cyclohexylethyl)-5-(2,6-dimethoxy-4-methylphenyl)-1H-1,2,4-triazol-3-yl]amino}carbonyl)-5-ethyl-1H-indol-1-yl]acetic Acid

1.8 ml (0.0018 mol) of 1 N sodium hydroxide solution are added to 530 mg (0.0009 mol) of methyl 2-[2-({[1-(2-cyclohexylethyl)-5-(2,6-dimethoxy-4-methylphenyl)-1H-1,2,4-triazol-3-yl]amino}carbonyl)-5-ethyl-1H-indol-1-yl] prepared according to Example 1, dissolved in 50 ml of methanol. After 18 hours at room temperature, the reaction mixture is evaporated to dryness. The residue is taken up in ethyl acetate and 0.5 N hydrochloric acid solution. The organic phase is separated out after settling has taken place, dried over anhydrous sodium sulphate and concentrated. The residue is purified by chromatography on a column of silica gel with the eluent: 92/8 (v/v) dichloromethane/methanol, to give white crystals; m.p.=198° C.; yield=91%.

Examples 3 to 511 described in Tables VI and VII below are prepared in the same way, by working according to Examples 1 and 2 above, starting with appropriate intermediates.

TABLE VI

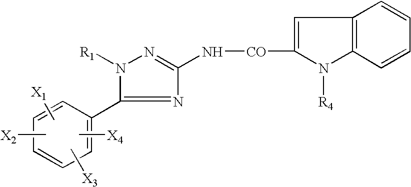

| EXAMPLE No. | 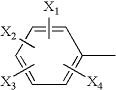 | R₁ | R₄ | m.p.: ° C. (salt) |
|---|---|---|---|---|
| 3 | 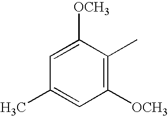 | 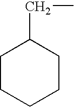 | —CH₂CO₂CH₃ | 185 |
| 4 | 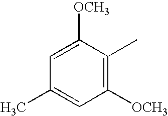 | 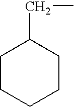 | —CH₂CO₂H | 226 |
| 5 | 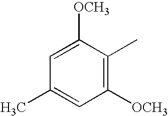 | 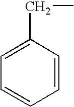 | —CH₂CO₂CH₃ | 118 |
| 6 | 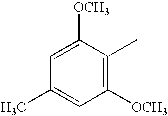 | 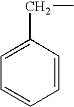 | —CH₂CO₂H | 230 |
| 7 | 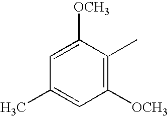 | 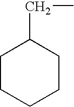 | —CH₂CH₂CO₂CH₃ | 101 |
| 8 | 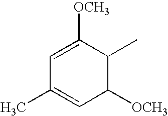 | —(CH₂)₄CH₃ | —CH₂CO₂CH₃ | 192 |
| 9 | 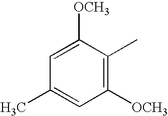 | 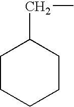 | —CH₂CH₂CO₂H | 210 |
| 10 | 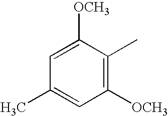 | —(CH₂)₄CH₃ | —CH₂CO₂H | 205 |
| 11 | 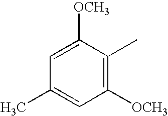 | 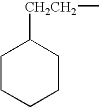 | —CH₂CO₂CH₃ | 189 |
| 12 | 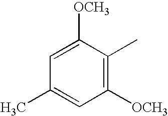 | 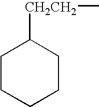 | —CH₂CO₂H | 218 |
| 13 | 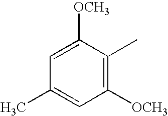 | 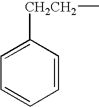 | —CH₂CO₂CH₃ | 138 |
| 14 | 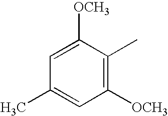 | 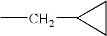 | —CH₂CO₂CH₃ | 115 |
| 15 | 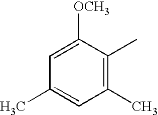 | 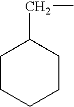 | —CH₂CO₂CH₃ | 167 |
| 16 | 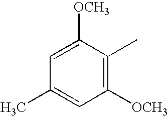 | 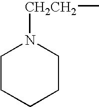 | —CH₂CO₂CH₃ | 180 |
| 17 | 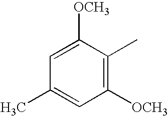 | 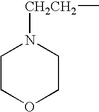 | —CH₂CO₂CH₃ | 203 |
| 18 | 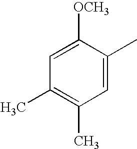 | 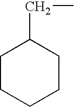 | —CH₂CO₂CH₃ | 158 |
| 19 | 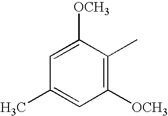 | 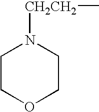 | —CH₂CO₂H | 217 (HCl) |
| 20 | 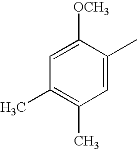 | 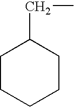 | —CH₂CO₂H | 168 |
| 21 | 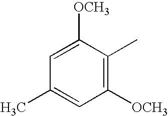 | 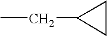 | —CH₂CO₂H | 271 |
| 22 | 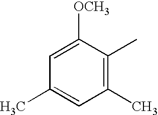 | 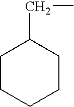 | —CH₂CO₂H | 181 |
| 23 | 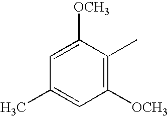 | —(CH₂)₃CH₃ | —CH₂CO₂CH₃ | 220 |
| 24 | 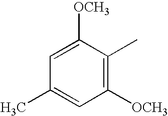 | 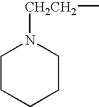 | —CH₂CO₂H | 220 (HCl) |
| 25 | 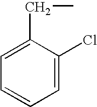 | 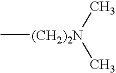 | —CH₂CO₂CH₃ | 165 |
| 26 | 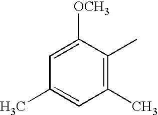 | 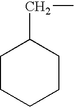 | —CH₂CO₂H | 198 |
| 27 | 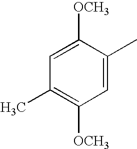 | 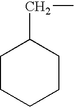 | —CH₂CO₂CH₃ | 132 |
| 28 | 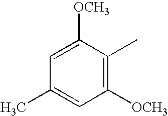 | —(CH₂)₃CH₃ | —CH₂CO₂H | 220 |
| 29 | 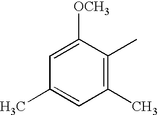 | 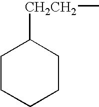 | —CH₂CO₂CH₃ | 144 |
| 30 | 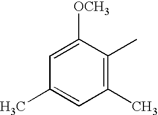 | 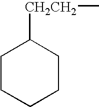 | —CH₂CO₂H | 169 |
| 31 | 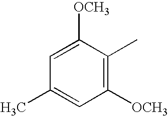 | 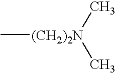 | —CH₂CO₂H | 203 (HCl) |
| 32 | 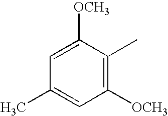 | 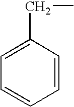 | —CH₂CO₂H | 180 |
| 33 | 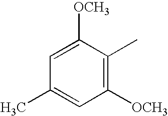 | 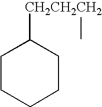 | —CH₂CO₂CH₃ | 172 |
| 34 | 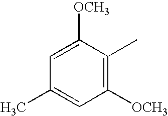 | 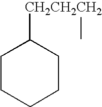 | —CH₂CO₂H | 216 |
| 35 | 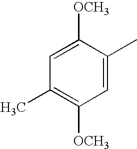 | 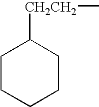 | —CH₂CO₂CH₃ | 128 |
| 36 | 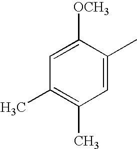 | 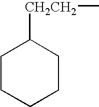 | —CH₂CO₂CH₃ | 158 |
| 37 | 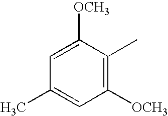 | —(CH₂)₂CH(CH₃)₂ | —CH₂CO₂H | 272 |
| 38 | 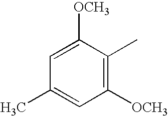 | —CH₂CH(CH₃)₂ | —CH₂CO₂CH₃ | 206 |
| 39 | 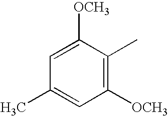 | —CH₂CH(CH₃)₂ | —CH₂CO₂H | 189 |
| 40 | 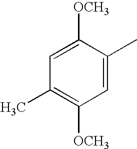 | 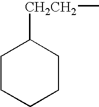 | —CH₂CO₂H | 175 |
| 41 | 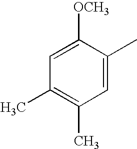 | 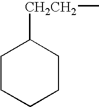 | —CH₂CO₂H | 158 |
| 42 | 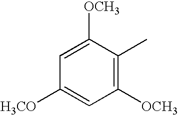 | 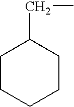 | —CH₂CO₂CH₃ | 180 |
| 43 | 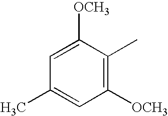 | 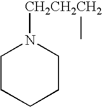 | —CH₂CO₂CH₃ | 161 |
| 44 | 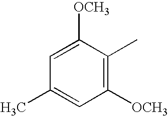 | 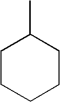 | —CH₂CO₂CH₃ | 210 |
| 45 | 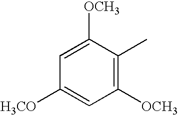 | 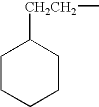 | —CH₂CO₂CH₃ | 191 |
| 46 | 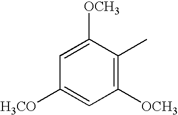 | 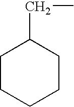 | —CH₂CO₂H | 182 |
| 47 | 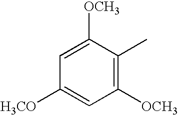 | 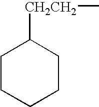 | —CH₂CO₂H | 195 |
| 48 | 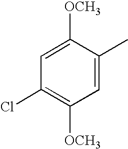 | 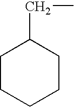 | —CH₂CO₂CH₃ | 201 |
| 49 | 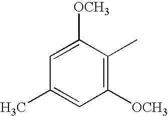 | 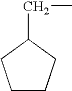 | —CH₂CO₂CH₃ | 194 |
| 50 | 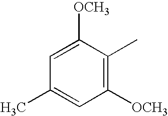 | 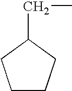 | —CH₂CO₂H | 204 |
| 51 | 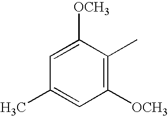 | 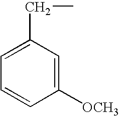 | —CH₂CO₂CH₃ | 129 |
| 52 | 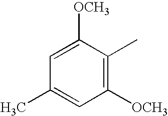 | 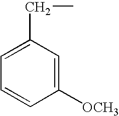 | —CH₂CO₂H | 213 |
| 53 | 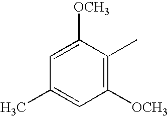 | 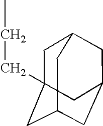 | —CH₂CO₂H | 182 |
| 54 | 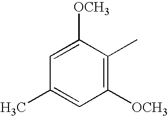 | 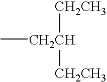 | —CH₂CO₂H | 151 |
| 55 | 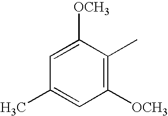 | 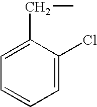 | —CH₂COOCH₃ | 192 |
| 56 | 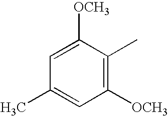 | 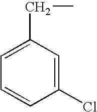 | —CH₂CO₂CH₃ | 173 |
| 57 | 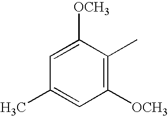 | 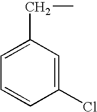 | —CH₂CO₂H | 229 |
| 58 | 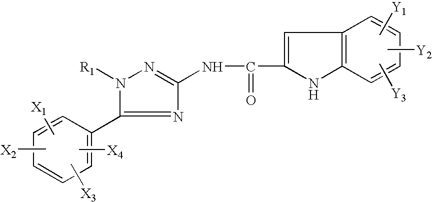 | 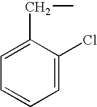 | —CH₂CO₂H | 195 |
| 59 | 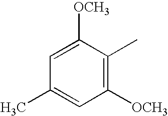 | 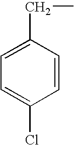 | —CH₂CO₂CH₃ | 133 |
| 60 | 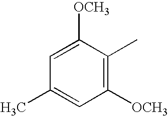 | 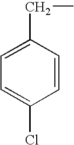 | —CH₂CO₂H | 175 |
| 61 | 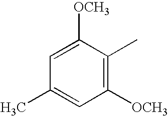 | 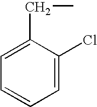 | —CH₂CH₂CO₂CH₃ | 178 |
| 62 | 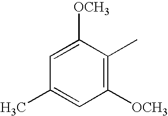 | 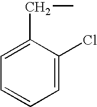 | —CH₂CH₂CO₂H | 235 |
| 63 | 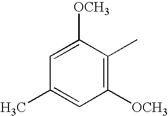 | 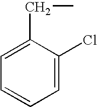 | —(CH₂)₃CO₂C₂H₅ | 144 |
| 64 | 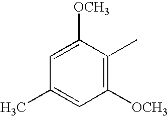 | 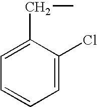 | —(CH₂)₃CO₂H | 141 |
| 65 | 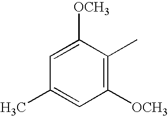 | 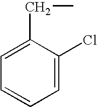 | —(CH₂)₄CO₂C₂H₅ | 95 |
| 66 | 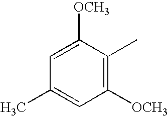 | 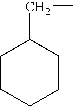 | —(CH₂)₄CO₂C₂H₅ | 101 |
| 67 | 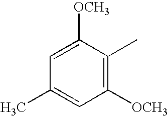 | 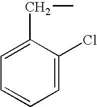 | —(CH₂)₄CO₂H | 266 |
| 68 | 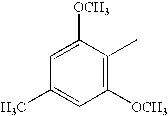 | 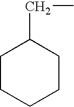 | —(CH₂)₄CO₂H | 157 |
| 69 | 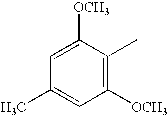 | 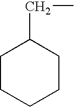 | —(CH₂)₃CO₂C₂H₅ | 114 |
| 70 | 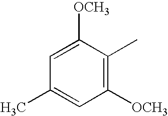 | 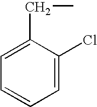 | —(CH₂)₃CO₂C₂H₅ | 76 |
| 71 | 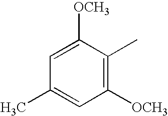 | 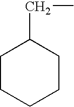 | —(CH₂)₃CO₂C₂H₅ | 85 |
| 72 | 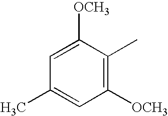 | 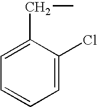 | —(CH₂)₃CO₂H | 243 |
| 73 | 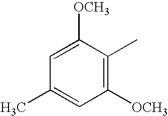 | 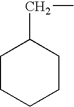 | —(CH₂)₃CO₂H | 138 |
| 74 | 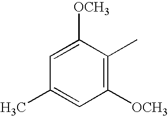 | 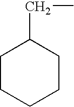 | —(CH₂)₃CO₂H | 150 |
| 75 | 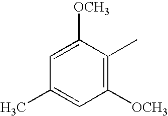 | 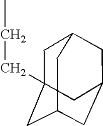 | —CH₂CO₂CH₃ | 201 |
| 76 | 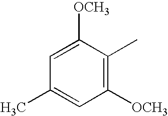 | 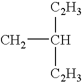 | —CH₂CO₂CH₃ | 162 |
| 77 | 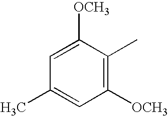 | 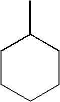 | —CH₂COOH | 200 |
| 78 | 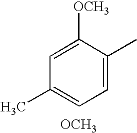 | 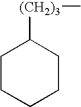 | —CH₂COOH | 168 (HCl) |
| 79 | 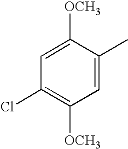 | 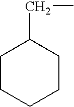 | —CH₂COOH | 211 |
| 80 | 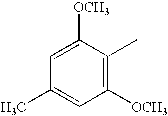 | 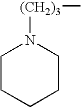 | —CH₂COOH | 243 (HCl) |
| 81 | 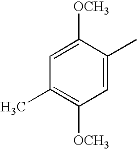 | 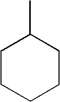 | —CH₂COOH | 188 (2HCl) |
| 82 | 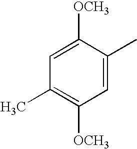 | 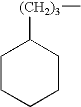 | —CH₂COOCH₃ | 200 |
| 83 | 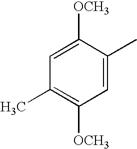 | 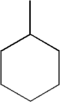 | —CH₂COOCH₃ | 170 |
| 84 | 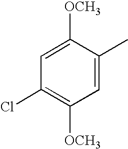 | 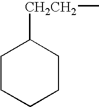 | —CH₂COOCH₃ | 137 |
| 85 | 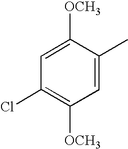 | 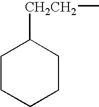 | —CH₂COOH | 168 |
| 86 | 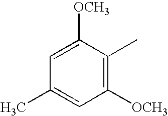 | 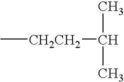 | —CH₂COOCH₃ | 156 |

TABLE VII

| | |
|---|---|
| | (I) |
| 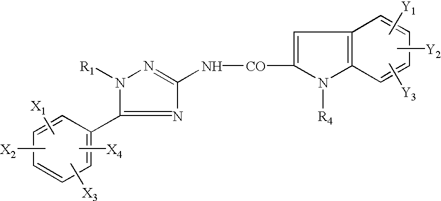 | |

| EXAMPLE No. | 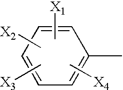 | R₁ | 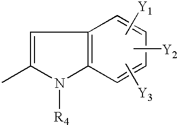 | m.p.: ° C. (salt) |
|---|---|---|---|---|
| 87 | 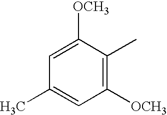 | 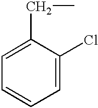 | 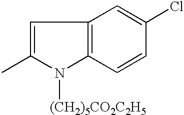 | 145 |
| 88 | 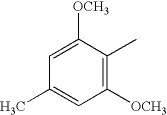 | 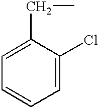 | 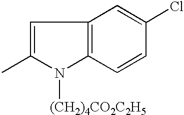 | 147 |
| 89 | 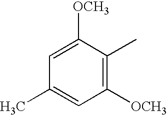 | 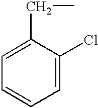 | 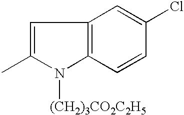 | 156 |
| 90 | 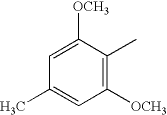 | 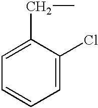 | 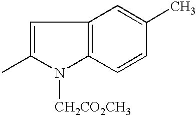 | 221 |
| 91 | 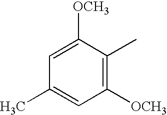 | 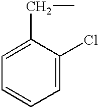 | 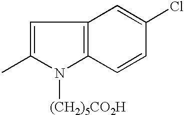 | 243 |
| 92 | 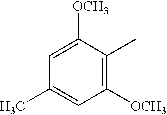 | 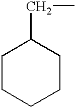 | 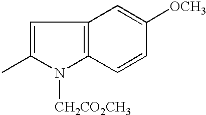 | 207 |
| 93 | 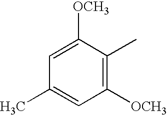 | 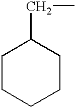 | 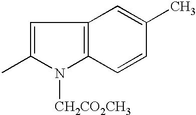 | 196 |
| 94 | 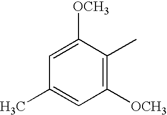 | 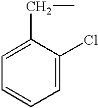 | 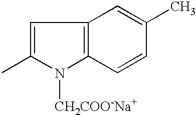 | 310 (Na salt) |
| 95 | 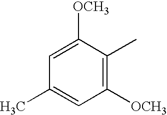 | 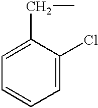 | 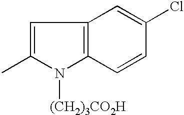 | 221 |
| 96 | 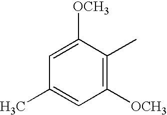 | 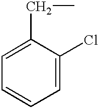 | 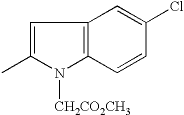 | 214 |
| 97 | 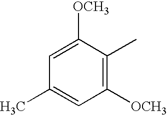 | 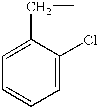 | 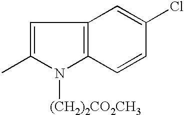 | 202 |
| 98 | 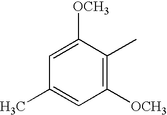 | 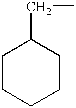 | 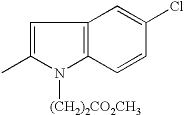 | 194 |
| 99 | 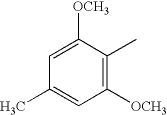 | 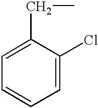 | 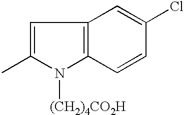 | 285 |
| 100 | 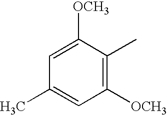 | 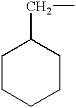 | 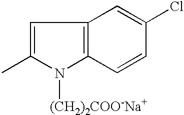 | 194 (Na salt) |
| 101 | 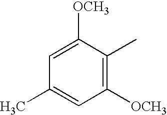 | 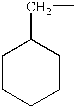 | 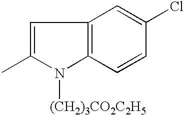 | 132 |
| 102 | 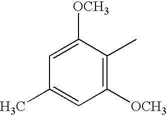 | 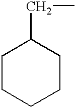 | 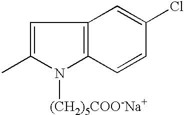 | 277 (Na salt) |
| 103 | 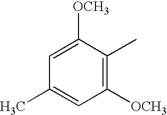 | 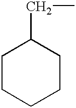 | 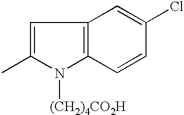 | 195 |
| 104 | 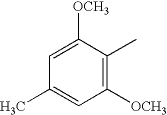 | 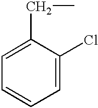 | 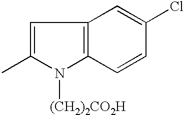 | 264 |
| 105 | 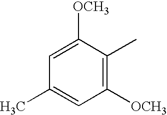 | 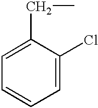 | 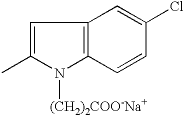 | 266 (Na salt) |
| 106 | 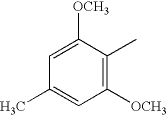 | 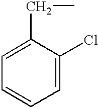 | 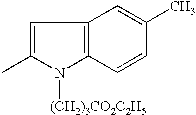 | 161 |
| 107 | 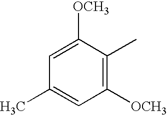 | 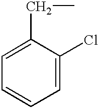 | 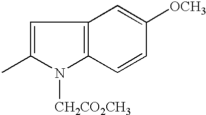 | 196 |
| 108 | 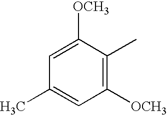 | 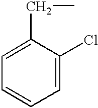 | 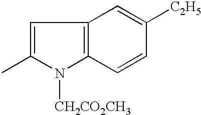 | 174 |
| 109 | 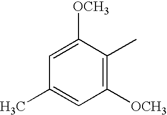 | 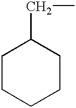 | 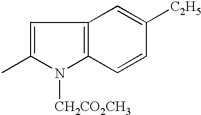 | 190 |
| 110 | 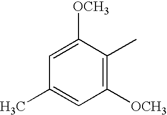 | 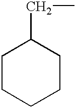 | 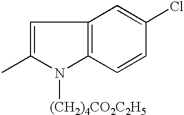 | 83 |
| 111 | 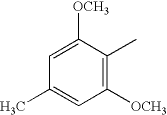 | 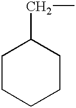 | 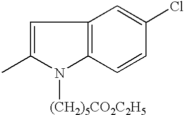 | 242 |
| 112 | 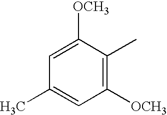 | 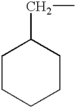 | 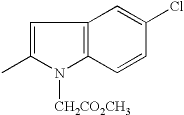 | 223 |
| 113 | 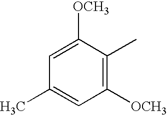 | 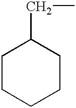 | 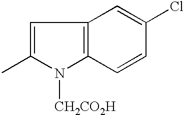 | 205 |
| 114 | 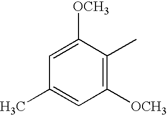 | 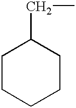 | 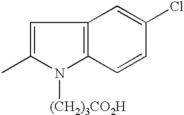 | 191 |
| 115 | 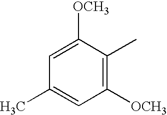 | 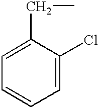 | 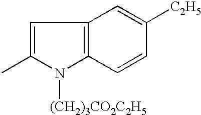 | 154 |
| 116 | 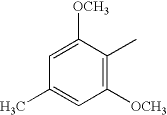 | 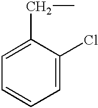 | 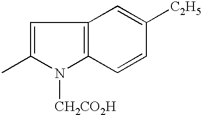 | 233 |
| 117 | 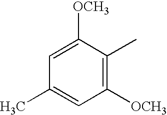 | 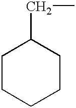 | 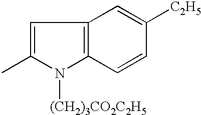 | 82 |
| 118 | 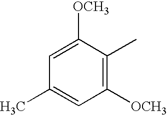 | 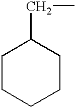 | 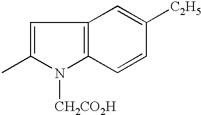 | 257 |
| 119 | 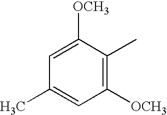 | 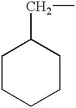 | 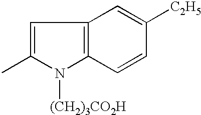 | 181 |
| 120 | 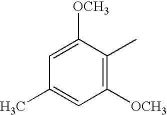 | 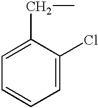 | 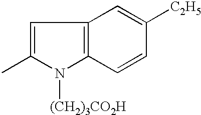 | 275 |
| 121 | 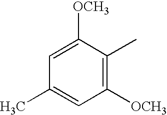 | 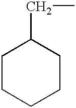 | 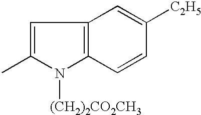 | 132 |
| 122 | 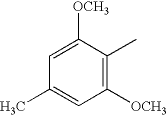 | 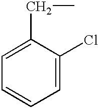 | 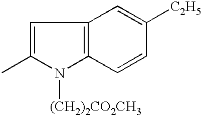 | 135 |
| 123 | 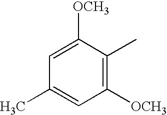 | 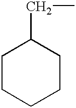 | 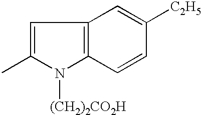 | 263 |
| 124 | 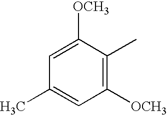 | 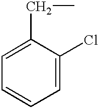 | 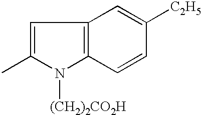 | 250 |
| 125 | 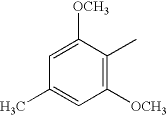 | 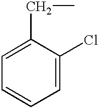 | 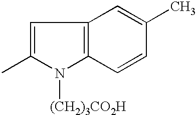 | 154 |
| 126 | 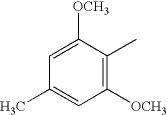 | 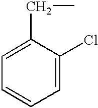 | 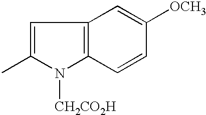 | 184 |
| 127 | 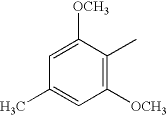 | 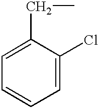 | 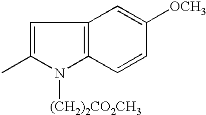 | 207 |
| 128 | 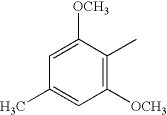 | 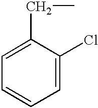 | 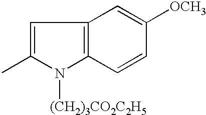 | 179 |
| 129 | 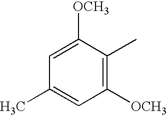 | 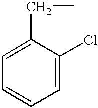 | 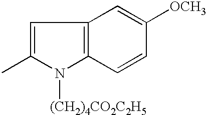 | 175 |
| 130 | 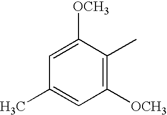 | 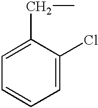 | 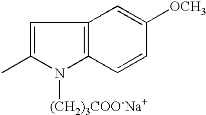 | 188 (Na salt) |
| 131 | 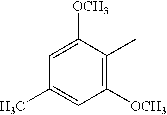 | 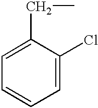 | 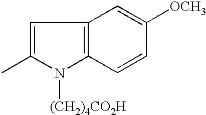 | 235 |
| 132 | 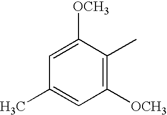 | 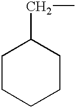 | 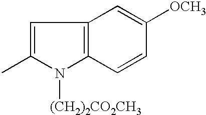 | 177 |
| 133 | 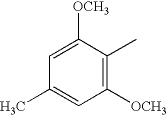 | 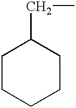 | 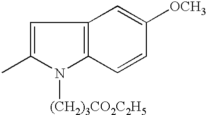 | 141 |
| 134 | 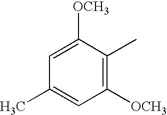 | 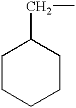 | 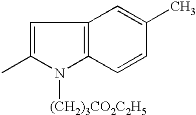 | 108 |
| 135 | 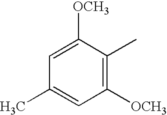 | 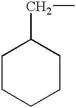 | 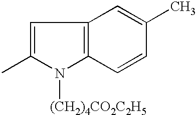 | 144 |
| 136 | 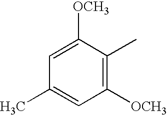 | 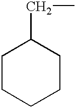 | 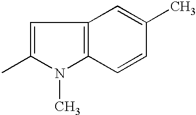 | 196 |
| 137 | 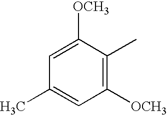 | 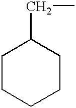 | 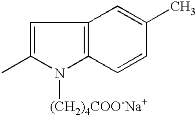 | 249 (Na salt) |
| 138 | 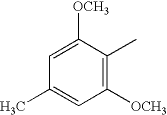 | 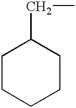 | 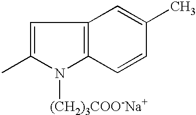 | 176 (Na salt) |
| 139 | 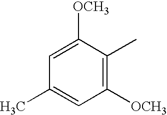 | 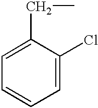 | 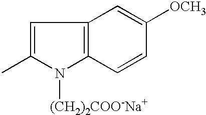 | 198 |
| 140 | 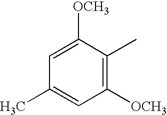 | 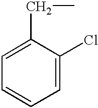 | 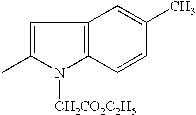 | 212 |
| 141 | 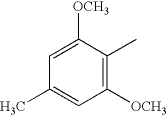 | 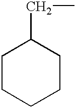 | 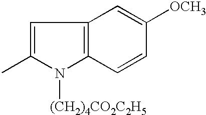 | 140 |
| 142 | 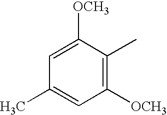 | 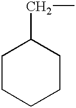 | 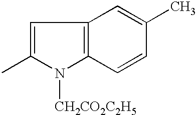 | 159 |
| 143 | 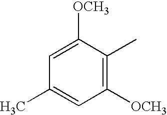 | 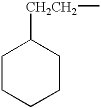 | 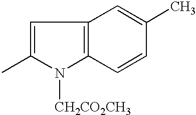 | 121 |
| 144 | 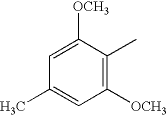 | 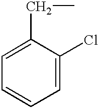 | 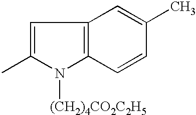 | 158 |
| 145 | 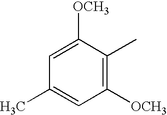 | 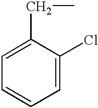 | 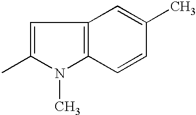 | 220 |
| 146 | 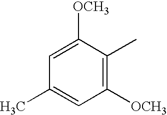 | 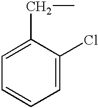 | 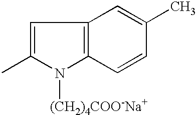 | 266 (Na salt) |
| 147 | 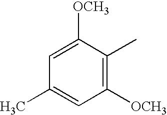 | 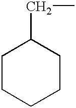 | 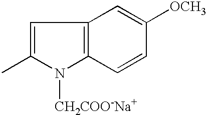 | 206 (Na salt) |
| 148 | 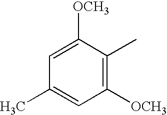 | 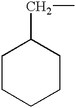 | 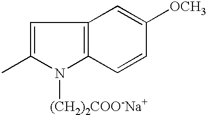 | 210 (Na salt) |
| 149 | 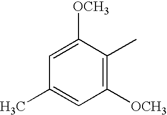 | 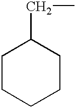 | 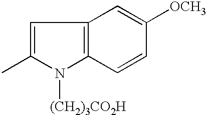 | 213 |
| 150 | 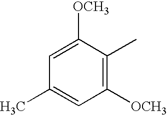 | 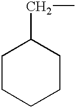 | 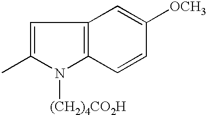 | 247 |
| 151 | 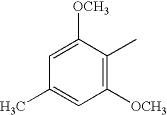 | 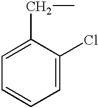 | 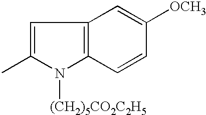 | 183 |
| 152 | 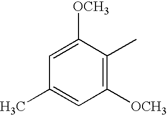 | 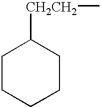 | 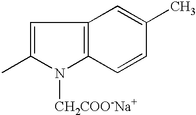 | 230 (Na salt) |
| 153 | 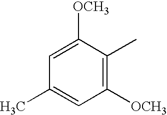 | 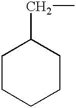 | 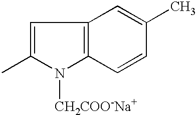 | 252 (Na salt) |
| 154 | 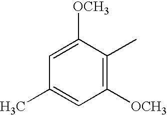 | 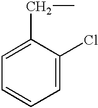 | 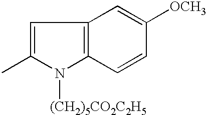 | 132 |
| 155 | 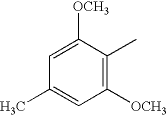 | 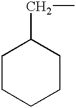 | 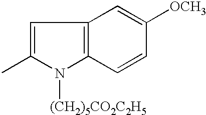 | 138 |
| 156 | 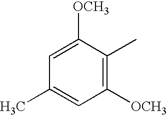 | 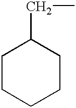 | 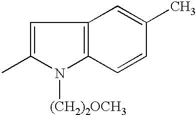 | 188 |
| 157 | 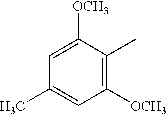 | 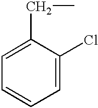 | 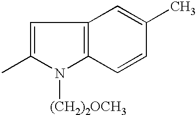 | 196 |
| 158 | 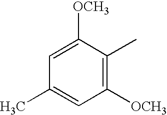 | 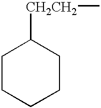 | 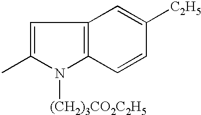 | 82 |
| 159 | 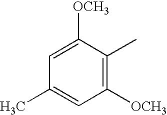 | 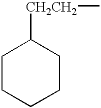 | 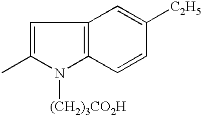 | 215 |
| 160 | 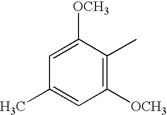 | 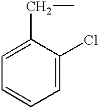 | 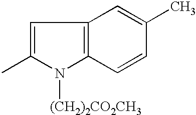 | 177 |
| 161 | 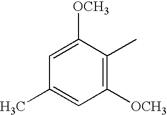 | 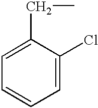 | 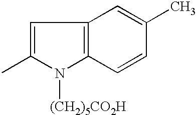 | 233 |
| 162 | 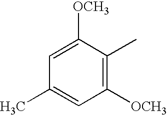 | 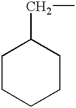 | 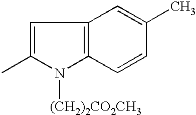 | 131 |
| 163 | 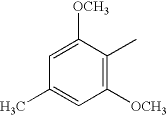 | 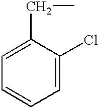 | 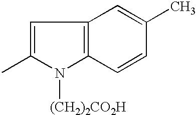 | 241 |
| 164 | 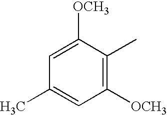 | 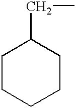 | 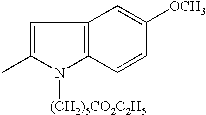 | 120 |
| 165 | 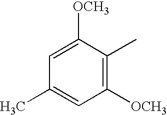 | 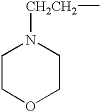 | 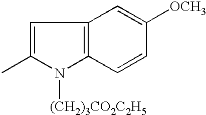 | 145 |
| 166 | 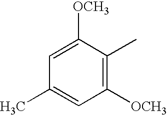 | 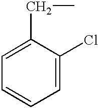 | 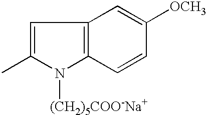 | 144 (Na salt) |
| 167 | 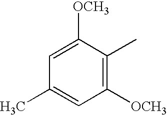 | 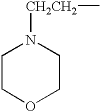 | 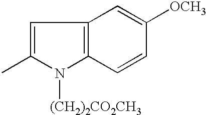 | 114 |
| 168 | 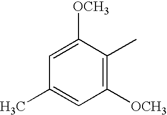 | 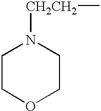 | 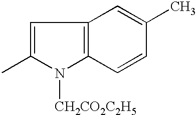 | 148 |
| 169 | 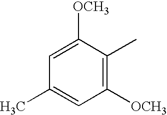 | 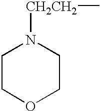 | 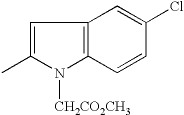 | 202 |
| 170 | 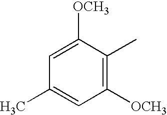 | 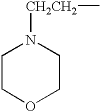 | 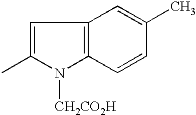 | 231 (HCl) |
| 171 | 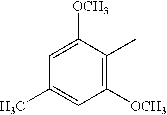 | 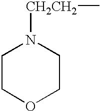 | 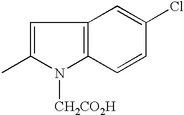 | 237 (2HCl) |
| 172 | 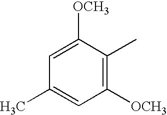 | 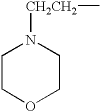 | 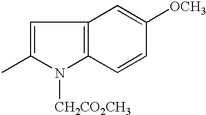 | 208 |
| 173 | 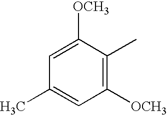 | 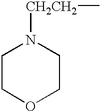 | 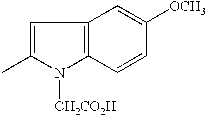 | 231 (2HCl) |
| 174 | 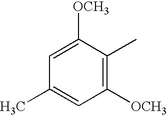 | 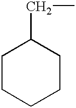 | 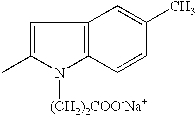 | 268 (Na salt) |
| 175 | 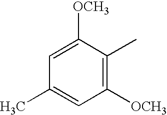 | 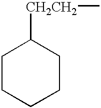 | 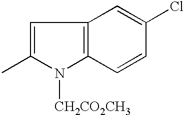 | 195 |
| 176 | 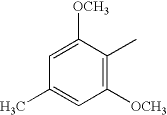 | 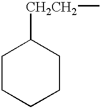 | 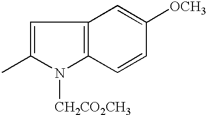 | 164 |
| 177 | 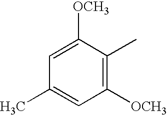 | 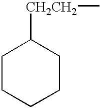 | 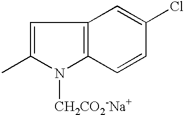 | 215 |
| 178 | 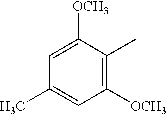 | 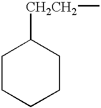 | 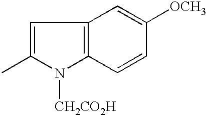 | 232 |
| 179 | 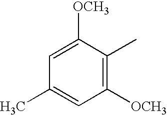 | 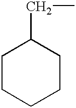 | 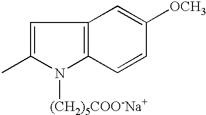 | 200 (Na salt) |
| 180 | 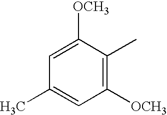 | 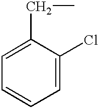 | 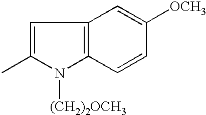 | 199 |
| 181 | 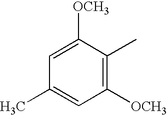 | 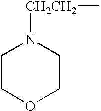 | 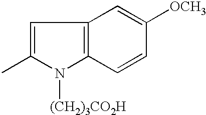 | 233 (HCl) |
| 182 | 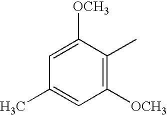 | 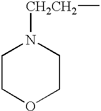 | 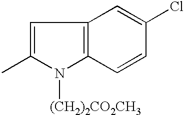 | 101 |
| 183 | 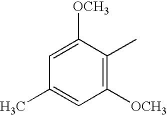 | 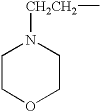 | 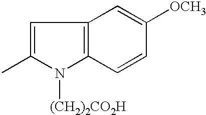 | 246 (HCl) |
| 184 | 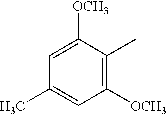 | 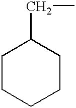 | 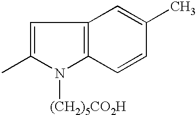 | 217 |
| 185 | 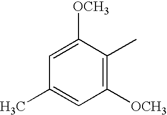 | 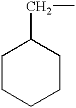 | 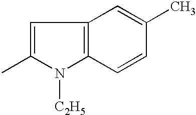 | 108 |
| 186 | 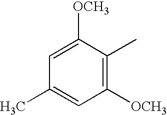 | 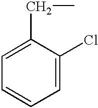 | 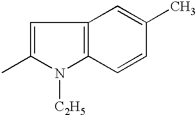 | 219 |
| 187 | 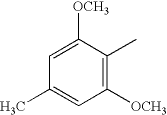 | 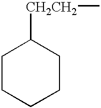 | 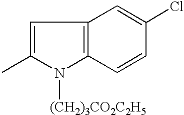 | 87 |
| 188 | 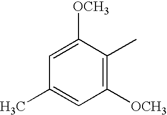 | 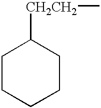 | 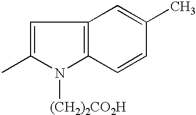 | 263 |
| 189 | 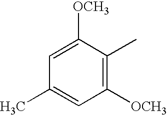 | 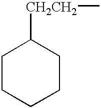 | 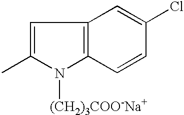 | 184 (Na salt) |
| 190 | 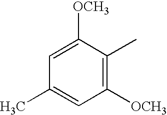 | 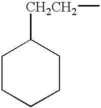 | 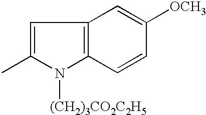 | 140 |
| 191 | 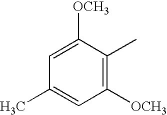 | 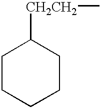 | 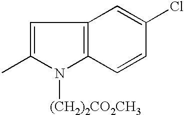 | 187 |
| 192 | 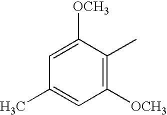 | 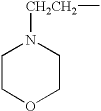 | 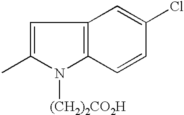 | 208 (HCl) |
| 193 | 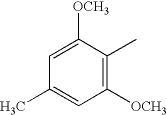 | 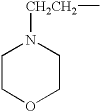 | 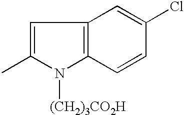 | 200 (HCl) |
| 194 | 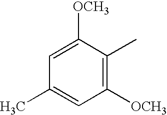 | 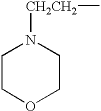 | 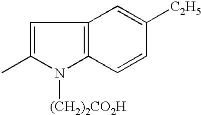 | 197 (2HCl) |
| 195 | 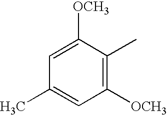 | 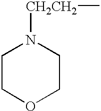 | 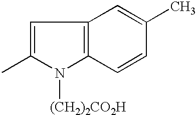 | 186 (2HCl) |
| 196 | 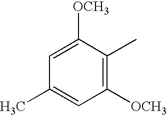 | 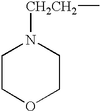 | 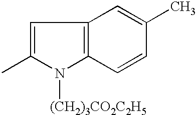 | 148 |
| 197 | 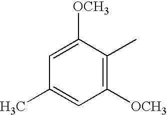 | 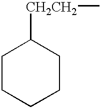 | 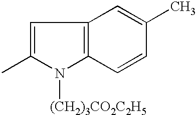 | 136 |
| 198 | 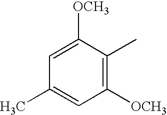 | 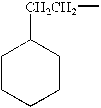 | 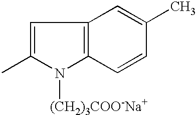 | 170 |
| 199 | 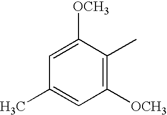 | 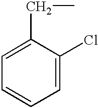 | 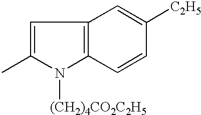 | 130 |
| 200 | 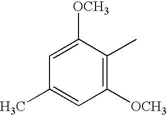 | 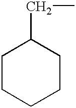 | 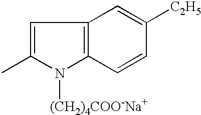 | 282 (Na salt) |
| 201 | 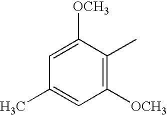 | 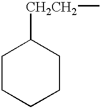 | 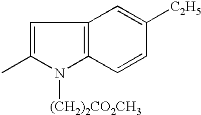 | 101 |
| 202 | 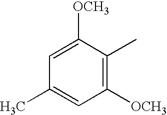 | 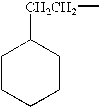 | 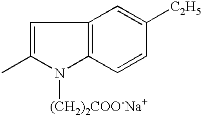 | 273 (Na salt) |
| 203 | 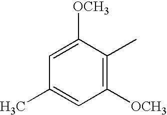 | 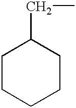 | 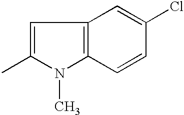 | 231 |
| 204 | 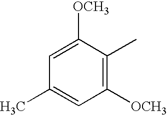 | 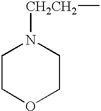 | 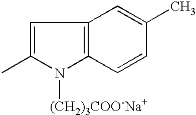 | 225 (Na salt) |
| 205 | 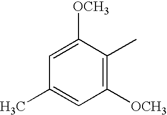 | 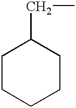 | 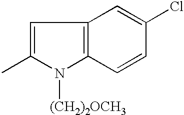 | 112 |
| 206 | 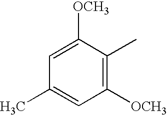 | 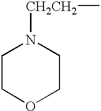 | 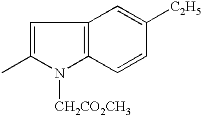 | 108 |
| 207 | 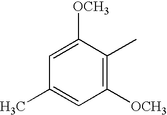 | 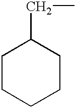 | 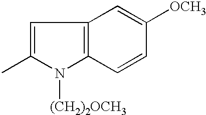 | 122 |
| 208 | 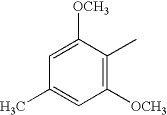 | 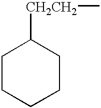 | 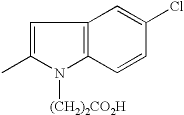 | 155 |
| 209 | 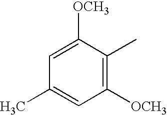 | 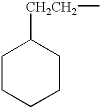 | 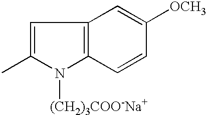 | 162 (Na salt) |
| 210 | 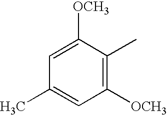 | 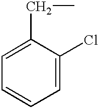 | 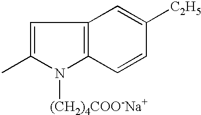 | 225 (Na salt) |
| 211 | 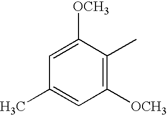 | 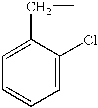 | 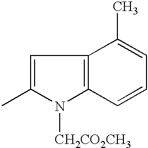 | 130 |
| 212 | 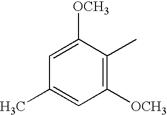 | 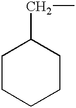 | 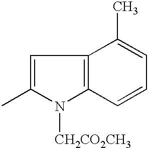 | 141 |
| 213 | 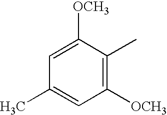 | 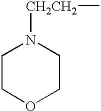 | 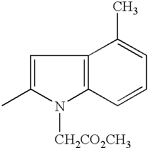 | 177 |
| 214 | 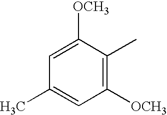 | 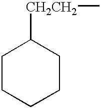 | 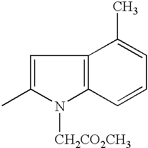 | 126 |
| 215 | 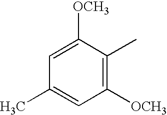 | 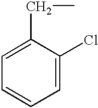 | 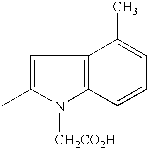 | 213 |
| 216 | 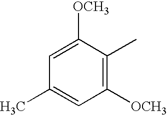 | 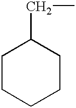 | 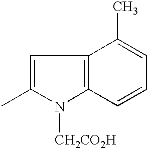 | 241 |
| 217 | 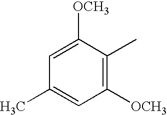 | 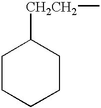 | 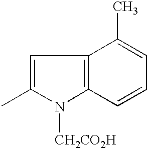 | 257 |
| 218 | 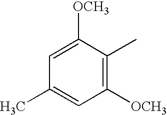 | 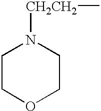 | 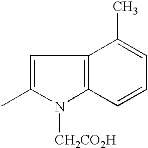 | 221 (2HCl) |
| 219 | 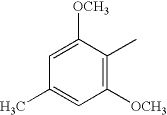 | 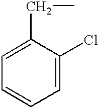 | 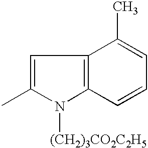 | 152 |
| 220 | 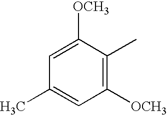 | 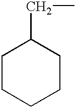 | 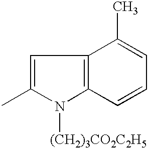 | 87 |
| 221 | 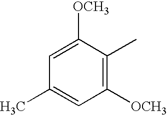 | 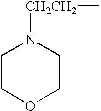 | 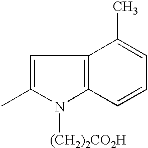 | 182 (2HCl) |
| 222 | 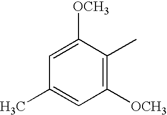 | 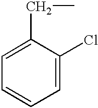 | 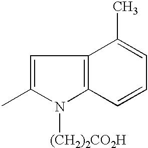 | 168 |
| 223 | 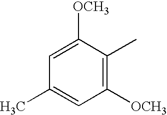 | 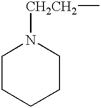 | 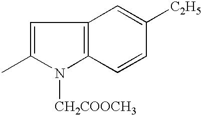 | 205 |
| 224 | 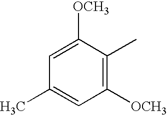 | 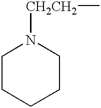 | 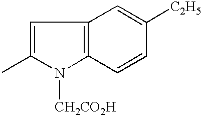 | 256 (HCl) |
| 225 | 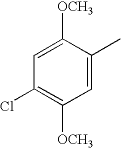 | 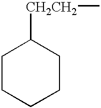 | 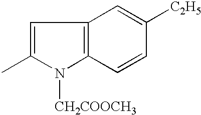 | 198 |
| 226 | 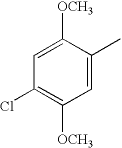 | 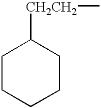 | 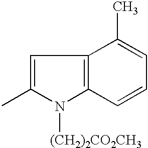 | 95 |
| 227 | 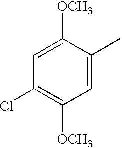 | 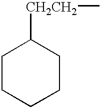 | 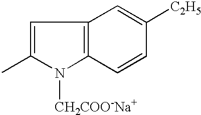 | 196 (Na salt) |
| 228 | 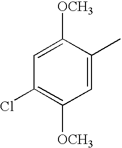 | 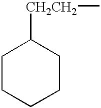 | 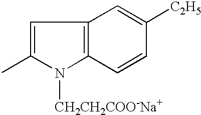 | 200 (Na salt) |
| 229 | 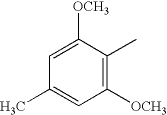 | 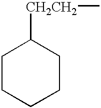 | 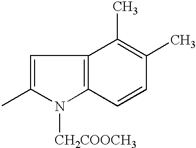 | 145 |
| 230 | 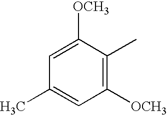 | 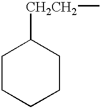 | 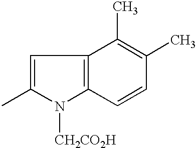 | 258 |
| 231 | 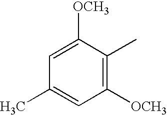 | 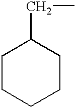 | 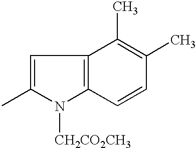 | 157 |
| 232 | 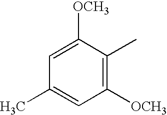 | 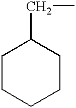 | 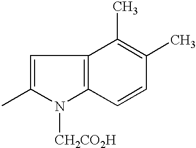 | 265 |
| 233 | 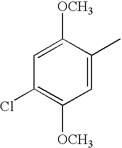 | 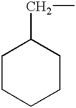 | 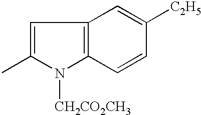 | 157 |
| 234 | 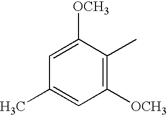 | 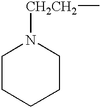 | 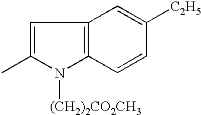 | 211 (HCl) |
| 235 | 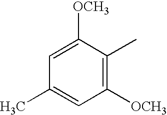 | 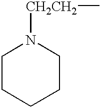 | 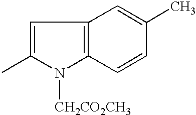 | 209 (HCl) |
| 236 | 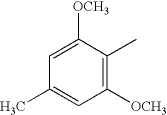 | 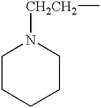 | 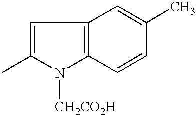 | 222 (2HCl) |
| 237 | 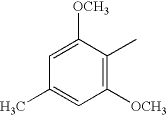 | 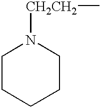 | 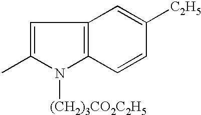 | 240 (HCl) |
| 238 | 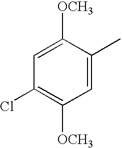 | 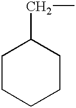 | 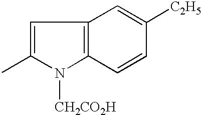 | 217 |
| 239 | 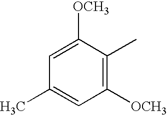 | 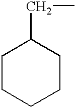 | 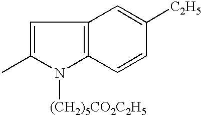 | 129 |
| 240 | 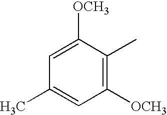 | 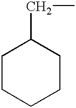 | 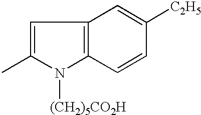 | 138 |
| 241 | 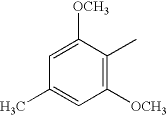 | 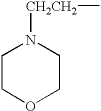 | 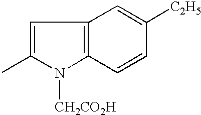 | 215 (2HCl) |
| 242 | 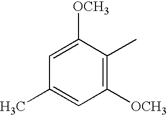 | 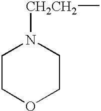 | 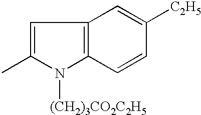 | 83 |
| 243 | 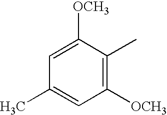 | 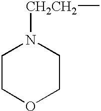 | 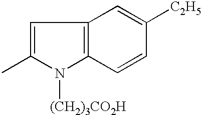 | 205 (2HCl) |
| 244 | 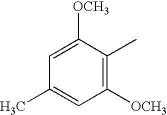 | 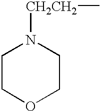 | 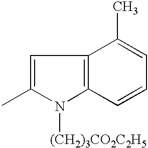 | 125 |
| 245 | 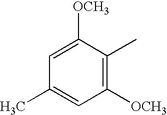 | 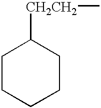 | 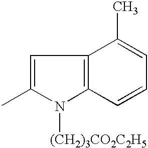 | 94 |
| 246 | 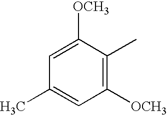 | 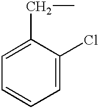 | 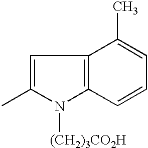 | 234 |
| 247 | 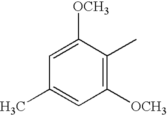 | 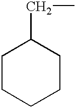 | 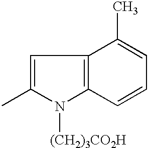 | 170 |
| 248 | 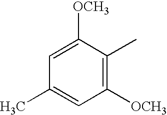 | 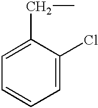 | 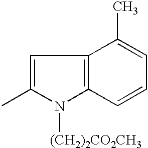 | 143 |
| 249 | 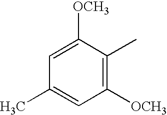 | 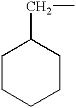 | 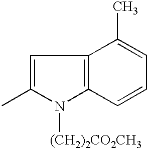 | 107 |
| 250 | 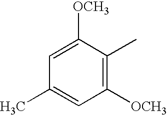 | 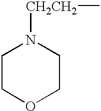 | 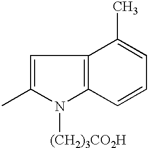 | 206 (2HCl) |
| 251 | 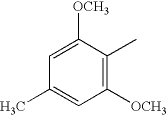 | 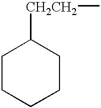 | 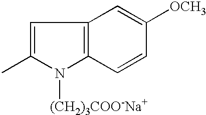 | 240 (Na salt) |
| 252 | 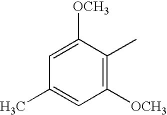 | 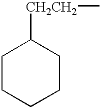 | 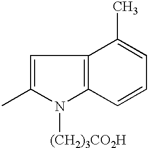 | 184 |
| 253 | 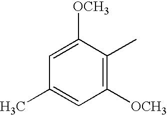 | 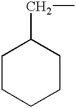 | 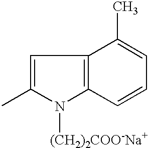 | 238 (Na salt) |
| 254 | 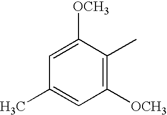 | 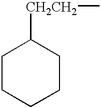 | 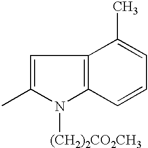 | 122 |
| 255 | 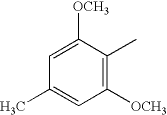 | 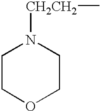 | 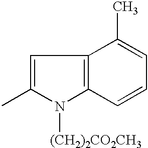 | 121 |
| 256 | 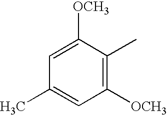 | 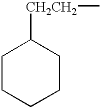 | 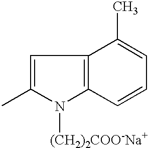 | 251 (Na salt) |
| 257 | 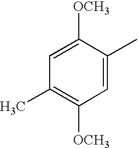 | 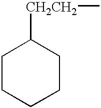 | 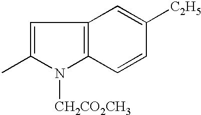 | 200 |
| 258 | 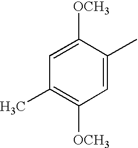 | 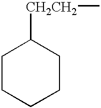 | 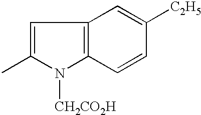 | 151 (2HCl) |
| 259 | 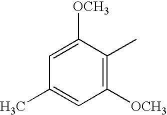 | 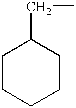 | 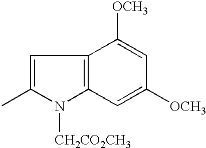 | 241 |
| 260 | 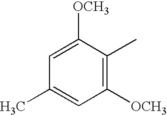 | 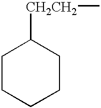 | 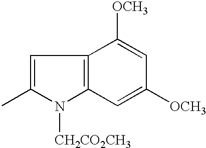 | 157 |
| 261 | 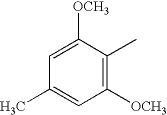 | 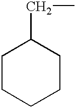 | 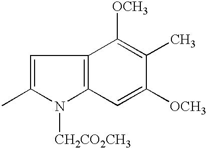 | 170 |
| 262 | 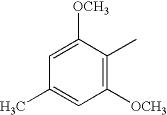 | 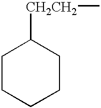 | 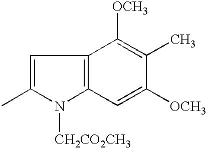 | 191 |
| 263 | 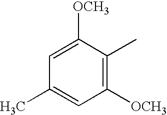 | 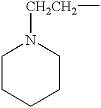 | 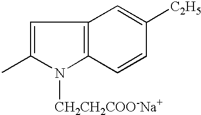 | 193 |
| 264 | 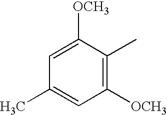 | 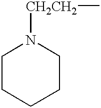 | 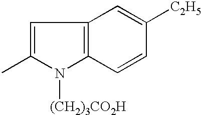 | 198 |
| 265 | 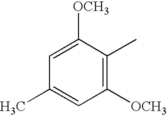 | 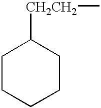 | 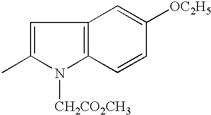 | 163 |
| 266 | 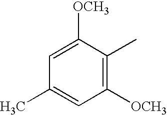 | 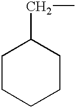 | 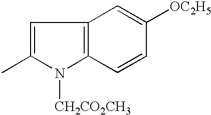 | 205 |
| 267 | 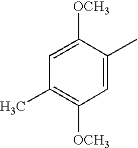 | 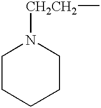 | 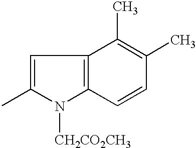 | 114 |
| 268 | 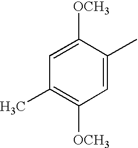 | 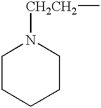 | 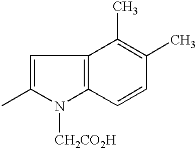 | 223 (HCl) |
| 269 | 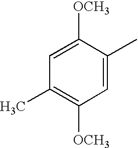 | 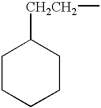 | 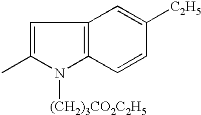 | 159 |
| 270 | 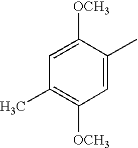 | 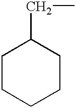 | 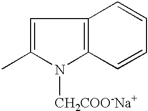 | 295 (Na salt) |
| 271 | 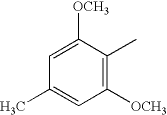 | 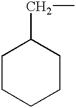 | 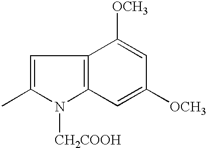 | 227 |
| 272 | 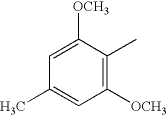 | 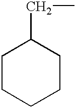 | 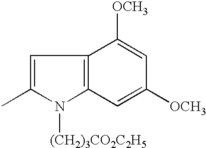 | 102 |
| 273 | 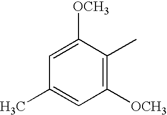 | 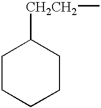 | 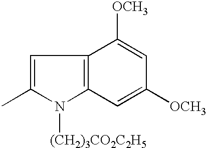 | 162 |
| 274 | 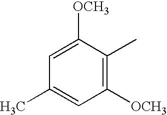 | 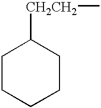 | 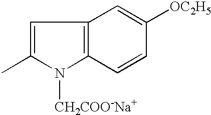 | 240 (Na salt) |
| 275 | 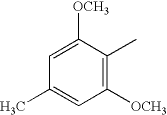 | 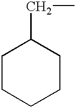 | 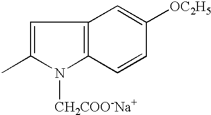 | 250 (Na salt) |
| 276 | 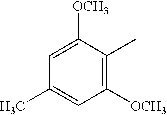 | 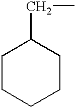 | 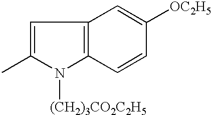 | 161 |
| 277 | 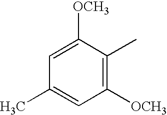 | 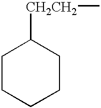 | 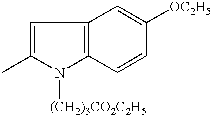 | 177 |
| 278 | 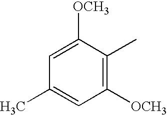 | 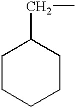 | 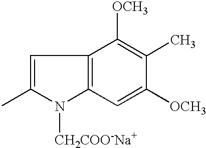 | 297 (Na salt) |
| 279 | 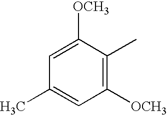 | 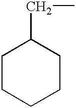 | 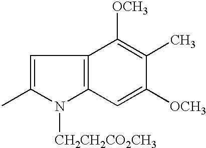 | 127 |
| 280 | 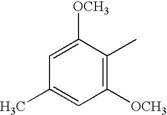 | 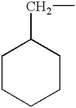 | 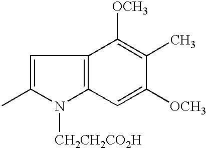 | 303 |
| 281 | 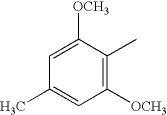 | 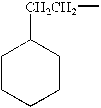 | 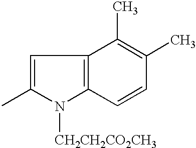 | 111 |
| 282 | 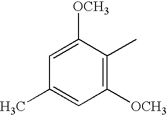 | 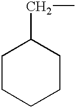 | 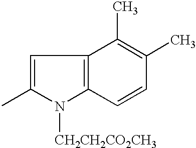 | 191 |
| 283 | 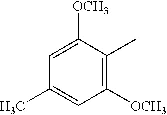 | 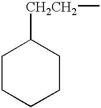 | 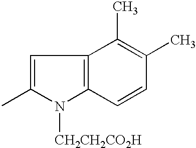 | 289 |
| 284 | 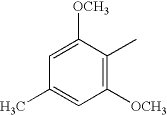 | 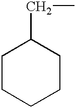 | 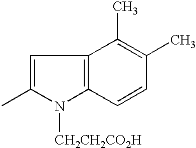 | 273 |
| 285 | 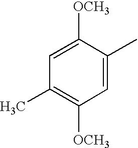 | 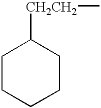 | 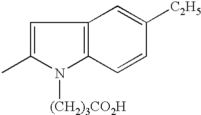 | 131 (HCl) |
| 286 | 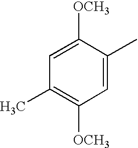 | 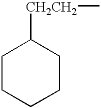 | 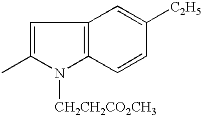 | 156 |
| 287 | 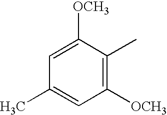 | 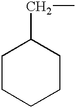 | 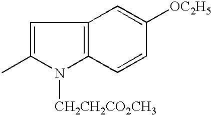 | 160 |
| 288 | 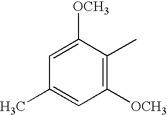 | 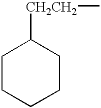 | 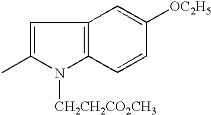 | 181 |
| 289 | 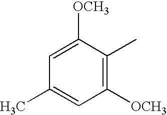 | 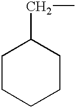 | 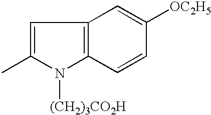 | 157 |
| 290 | 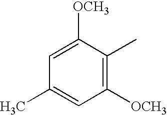 | 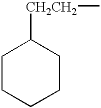 | 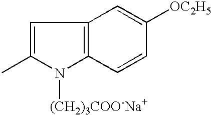 | 140 (Na salt) |
| 291 | 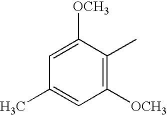 | 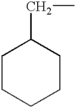 | 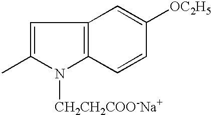 | 174 (Na salt) |
| 292 | 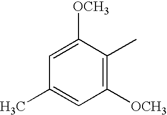 | 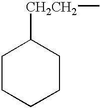 | 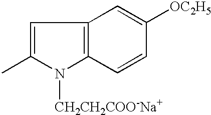 | 170 (Na salt) |
| 293 | 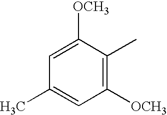 | 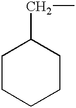 | 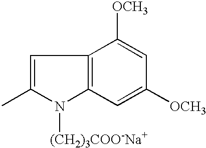 | 247 (Na salt) |
| 294 | 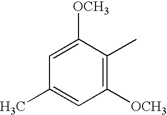 | 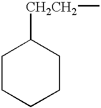 | 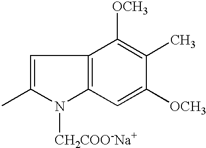 | 220 (Na salt) |
| 295 | 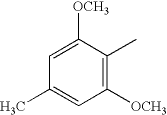 | 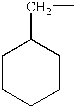 | 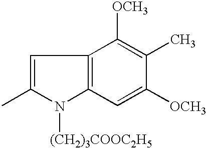 | 151 |
| 296 | 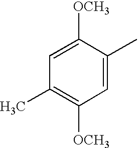 | 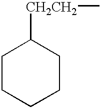 | 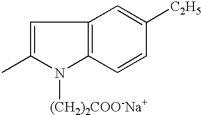 | 216 (Na salt) |
| 297 | 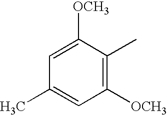 | 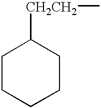 | 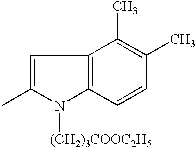 | 104 |
| 298 | 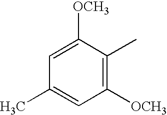 | 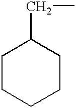 | 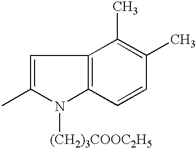 | 111 |
| 299 | 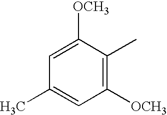 | 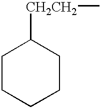 | 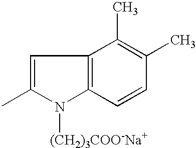 | 159 (Na salt) |
| 300 | 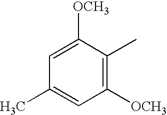 | 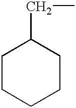 | 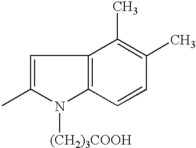 | 218 |
| 301 | 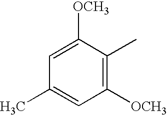 | 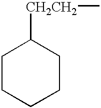 | 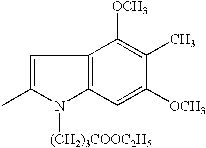 | 142 |
| 302 | 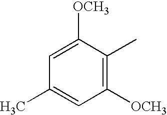 | 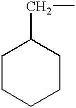 | 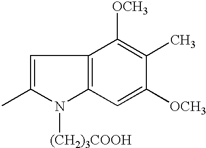 | 254 |
| 303 | 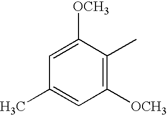 | 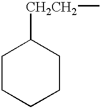 | 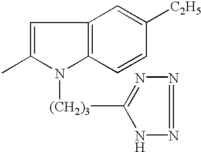 | 240 |
| 304 | 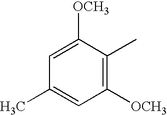 | 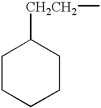 | 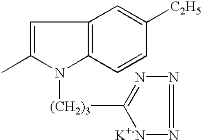 | 204 (K salt) |
| 305 | 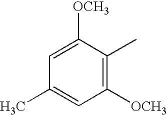 | 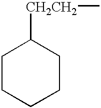 | 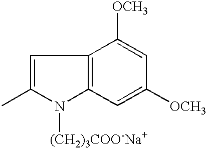 | 262 (Na salt) |
| 306 | 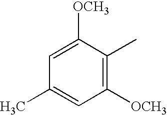 | 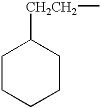 | 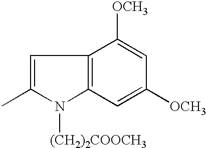 | 169 |
| 307 | 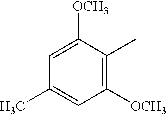 | 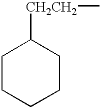 | 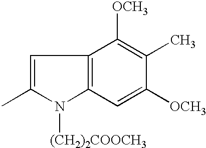 | 103 |
| 308 | 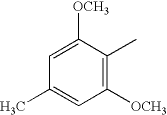 | 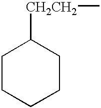 | 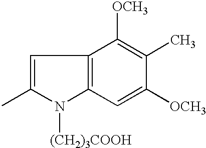 | 242 |
| 309 | 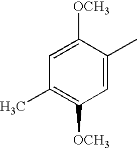 | 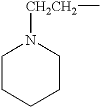 | 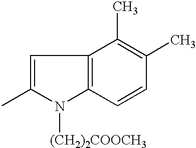 | 104 |
| 310 | 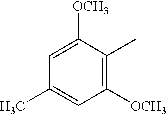 | 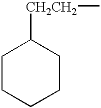 | 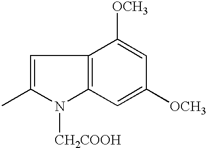 | 235 (HCl) |
| 311 | 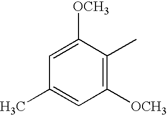 | 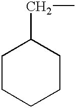 | 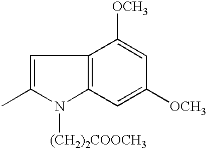 | 196 |
| 312 | 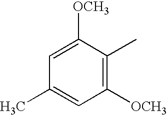 | 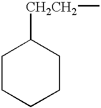 | 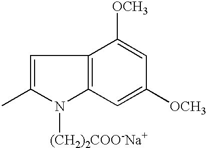 | 259 (Na salt) |
| 313 | 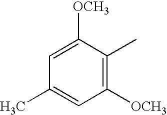 | 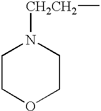 | 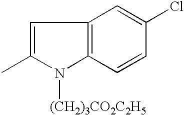 | 130 |
| 314 | 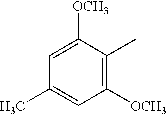 | 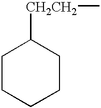 | 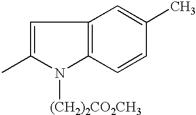 | 92 |
| 315 | 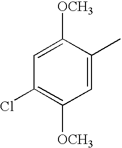 | 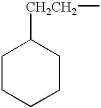 | 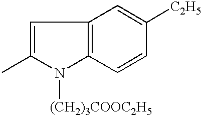 | 170 |
| 316 | 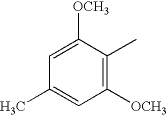 | 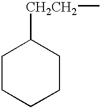 | 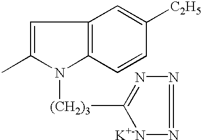 | 187 (K salt) |
| 317 | 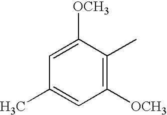 | 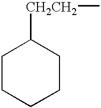 | 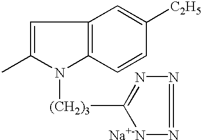 | 260 (Na salt) |
| 318 | 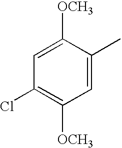 | 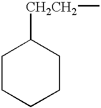 | 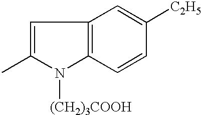 | 132 |
| 319 | 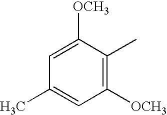 | 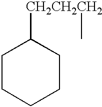 | 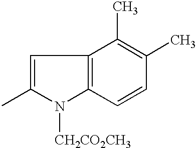 | 112 |
| 320 | 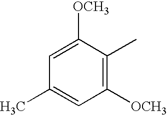 | 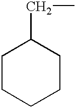 | 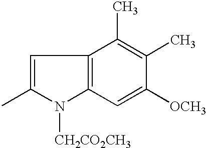 | 258 |
| 321 | 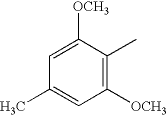 | 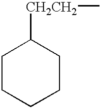 | 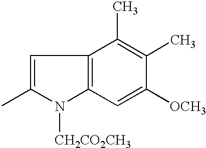 | 188 |
| 322 | 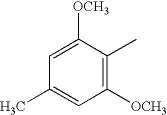 | 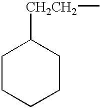 | 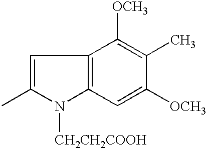 | 293 |
| 323 | 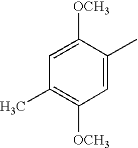 | 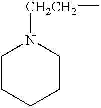 | 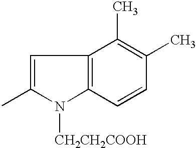 | 258 2HCl |
| 324 | 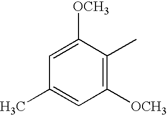 | 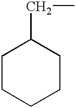 | 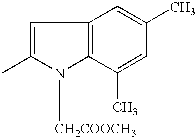 | 149 |
| 325 | 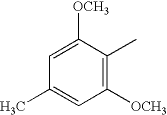 | 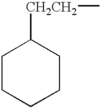 | 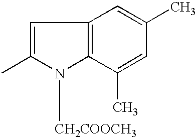 | 118 |
| 326 | 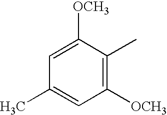 | 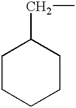 | 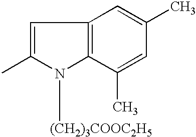 | 97 |
| 327 | 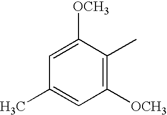 | 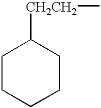 | 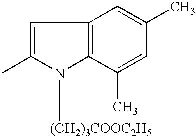 | 138 |
| 328 | 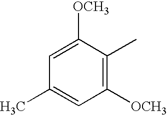 | 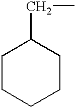 | 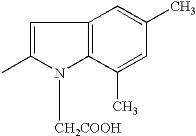 | 179 |
| 329 | 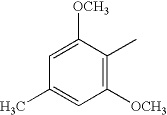 | 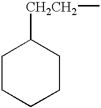 | 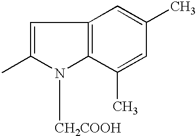 | 189 |
| 330 | 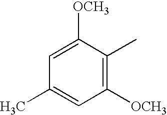 | 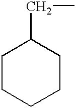 | 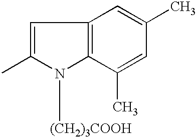 | 200 |
| 331 | 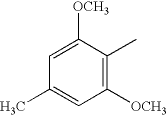 | 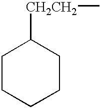 | 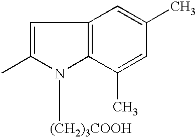 | 151 |
| 332 | 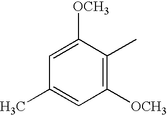 | 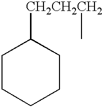 | 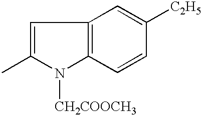 | 119 |
| 333 | 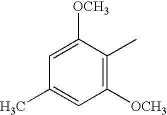 | 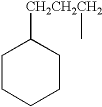 | 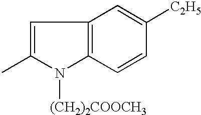 | 102 |
| 334 | 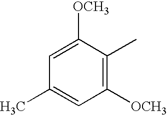 | 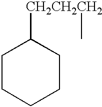 | 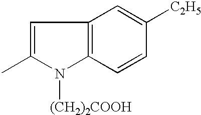 | 143 |
| 335 | 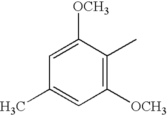 | 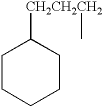 | 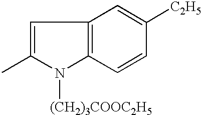 | 135 |
| 336 | 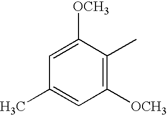 | 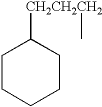 | 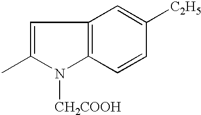 | 151 |
| 337 | 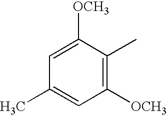 | 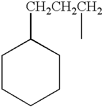 | 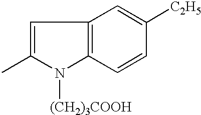 | 138 |
| 338 | 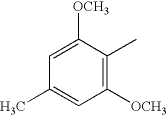 | 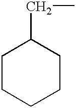 | 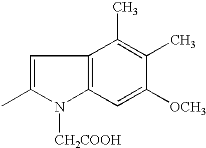 | 195 (HCl) |
| 339 | 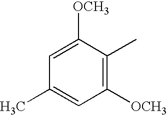 | 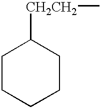 | 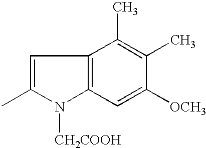 | 185 |
| 340 | 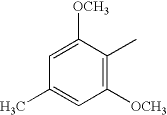 | 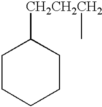 | 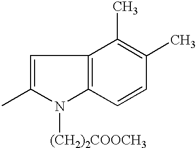 | 116 |
| 341 | 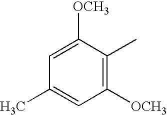 | 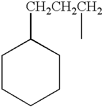 | 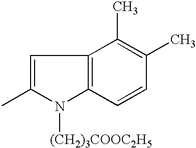 | 98 |
| 342 | 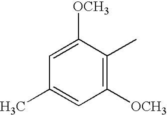 | 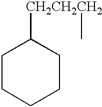 | 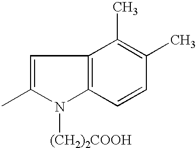 | 149 |
| 343 | 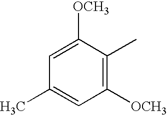 | 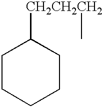 | 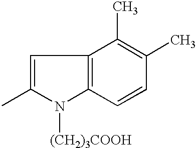 | 146 |
| 344 | 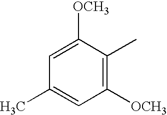 | 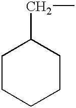 | 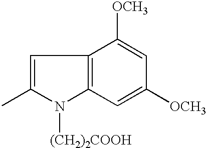 | 273 (HCl) |
| 345 | 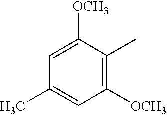 | 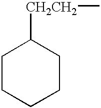 | 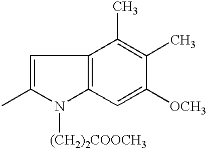 | 202 |
| 346 | 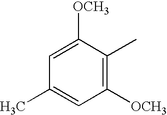 | 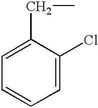 | 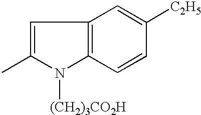 | 167 |
| 347 | 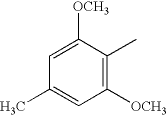 | 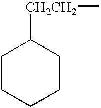 | 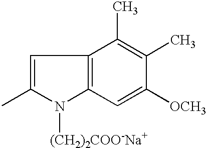 | 279 (Na salt) |
| 348 | 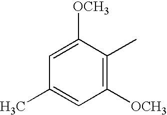 | 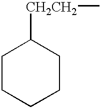 | 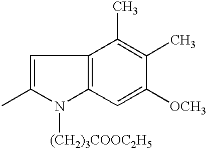 | 80 |
| 349 | 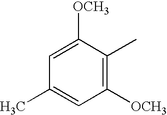 | 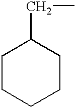 | 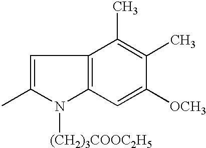 | 134 |
| 350 | 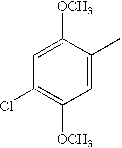 | 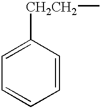 | 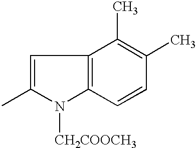 | 130 |
| 351 | 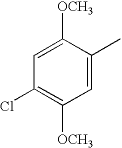 | 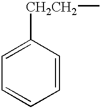 | 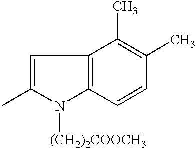 | 122 |
| 352 | 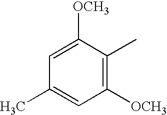 | 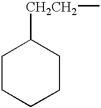 | 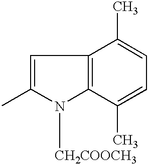 | 99 |
| 353 | 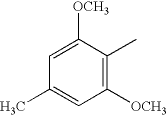 | 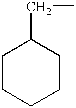 | 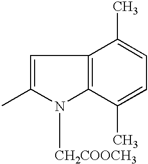 | 218 |
| 354 | 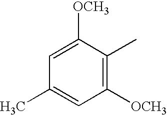 | 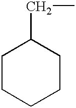 | 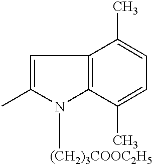 | 96 |
| 355 | 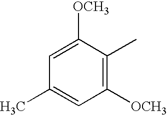 | 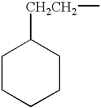 | 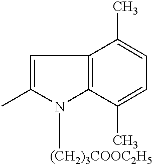 | 168 |
| 356 | 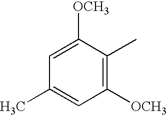 | 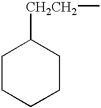 | 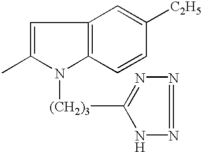 | 248 |
| 357 | 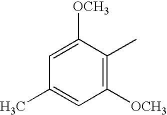 | 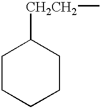 | 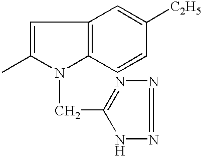 | 196 |
| 358 | 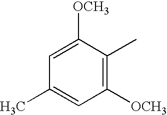 | 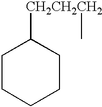 | 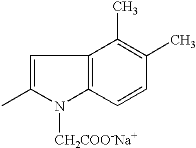 | 174 (Na salt) |
| 359 | 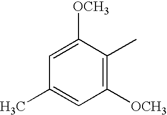 | 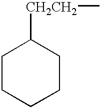 | 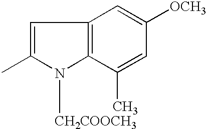 | 198 |
| 360 | 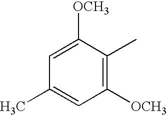 | 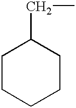 | 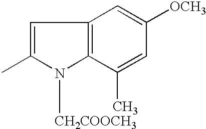 | 186 |
| 361 | 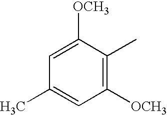 | 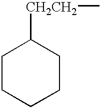 | 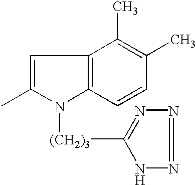 | 233 |
| 362 | 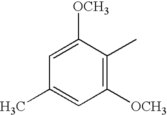 | 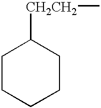 | 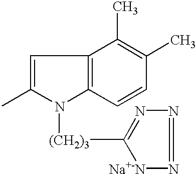 | 216 (Na salt) |
| 363 | 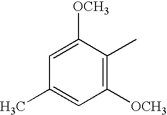 | 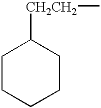 | 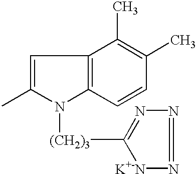 | 191 (K salt) |
| 364 | 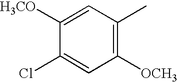 | 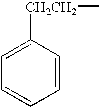 | 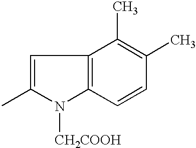 | 240 |
| 365 | 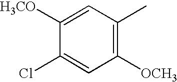 | 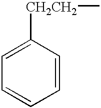 | 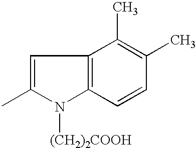 | 198 |
| 366 | 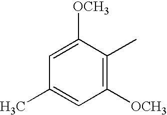 | 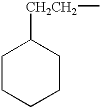 | 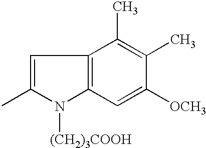 | 247 (HCl) |
| 367 | 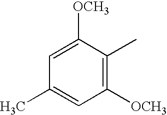 | 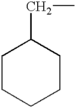 | 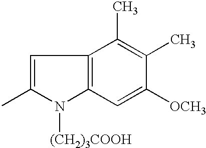 | 185 (HCl) |
| 368 | 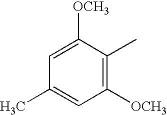 | 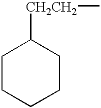 | 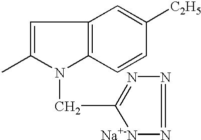 | 165 (Na salt) |
| 369 | 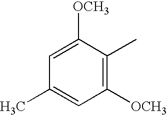 | 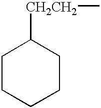 | 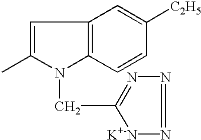 | 175 (K salt) |
| 370 | 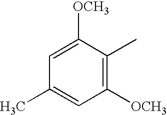 | 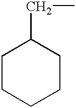 | 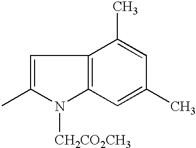 | 226 |
| 371 | 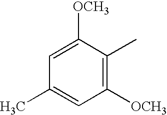 | 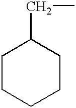 | 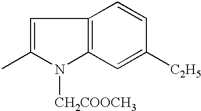 | 204 |
| 372 | 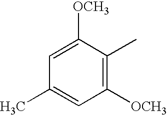 | 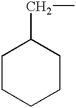 | 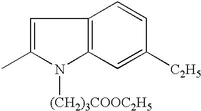 | 74 |
| 373 | 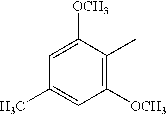 | 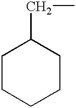 | 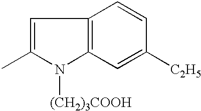 | 147 |
| 374 | 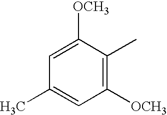 | 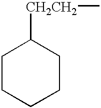 | 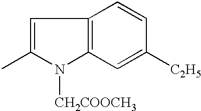 | 194 |
| 375 | 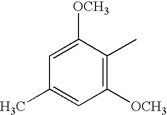 | 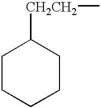 | 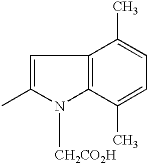 | 199 |
| 376 | 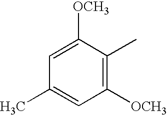 | 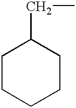 | 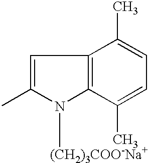 | 214 (HCl and Na salt) |
| 377 | 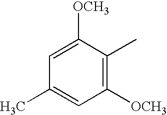 | 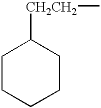 | 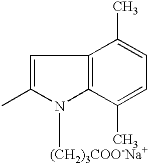 | 147 (HCl and Na salt) |
| 378 | 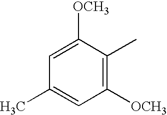 | 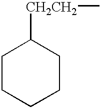 | 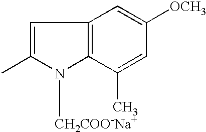 | 156 (Na salt) |
| 379 | 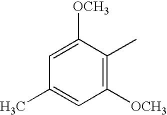 | 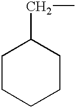 | 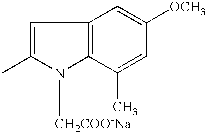 | 219 (Na salt) |
| 380 | 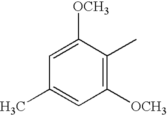 | 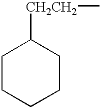 | 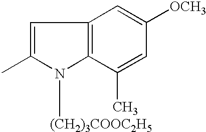 | 131 |
| 381 | 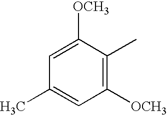 | 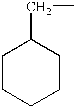 | 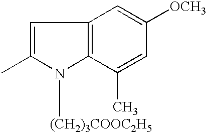 | 148 |
| 382 | 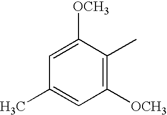 | 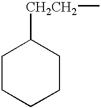 | 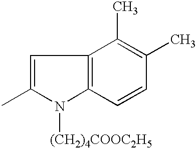 | 85 |
| 383 | 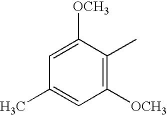 | 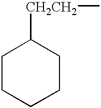 | 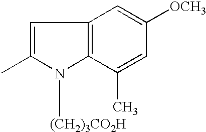 | 141 |
| 384 | 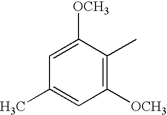 | 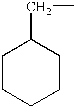 | 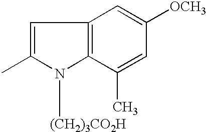 | 161 |
| 385 | 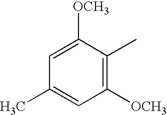 | 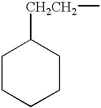 | 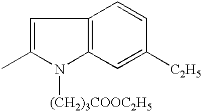 | 151 |
| 386 | 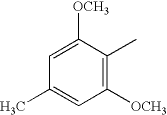 | 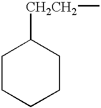 | 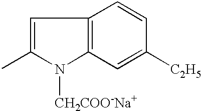 | 268 (Na salt) |
| 387 | 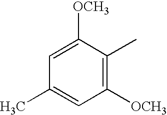 | 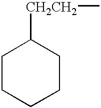 | 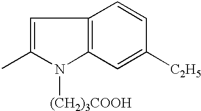 | 155 |
| 388 | 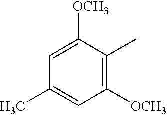 | 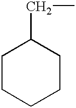 | 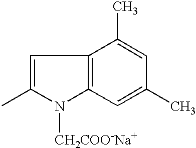 | 195 (Na salt) |
| 389 | 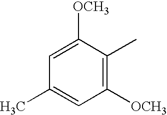 | 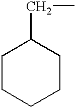 | 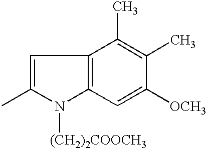 | 214 |
| 390 | 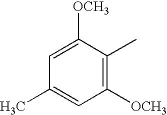 | 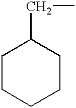 | 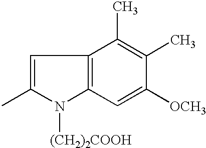 | 293 (HCl) |
| 391 | 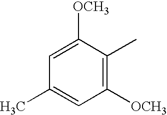 | 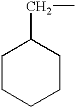 | 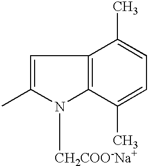 | 271 |
| 392 | 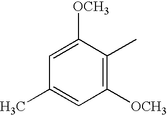 | 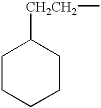 | 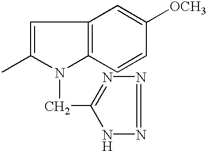 | 177 |
| 393 | 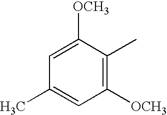 | 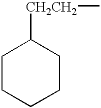 | 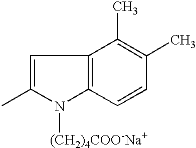 | 264 (Na salt) |
| 394 | 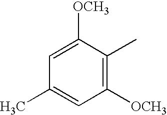 | 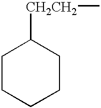 | 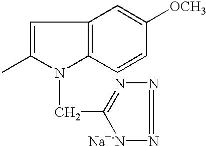 | 281 (Na salt) |
| 395 | 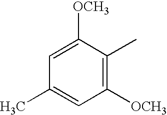 | 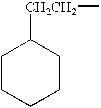 | 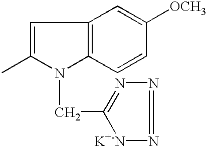 | 257 (K salt) |
| 396 | 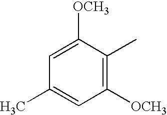 | 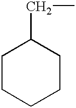 | 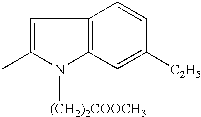 | 107 |
| 397 | 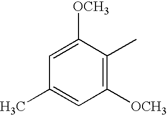 | 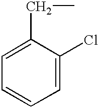 | 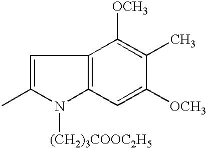 | 124 |
| 398 | 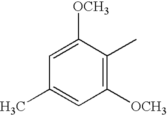 | 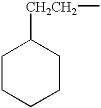 | 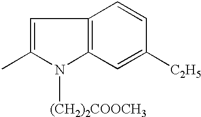 | 166 |
| 399 | 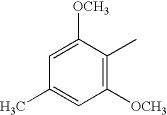 | 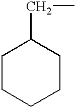 | 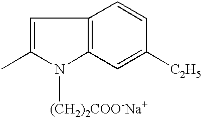 | 220 (Na salt) |
| 400 | 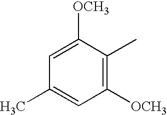 | 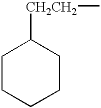 | 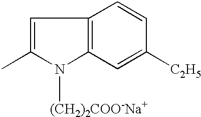 | 246 |
| 401 | 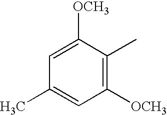 | 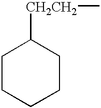 | 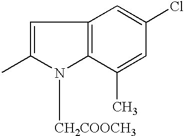 | 202 |
| 402 | 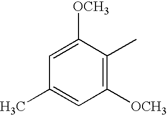 | 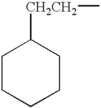 | 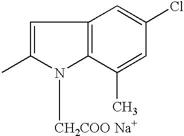 | 266 (Na salt) |
| 403 | 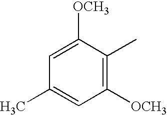 | 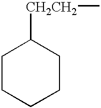 | 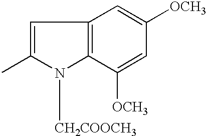 | 128 |
| 404 | 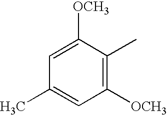 | 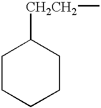 | 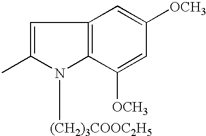 | 144 |
| 405 | 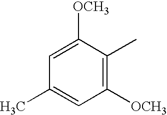 | 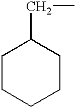 | 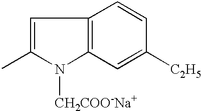 | 224 (Na salt) |
| 406 | 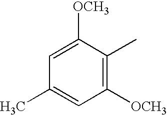 | 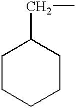 | 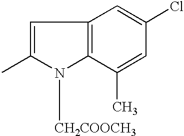 | 158 |
| 407 | 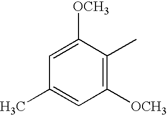 | 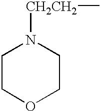 | 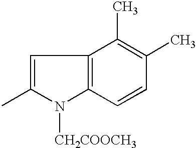 | 117 |
| 408 | 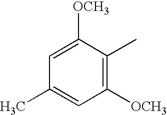 | 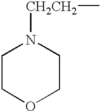 | 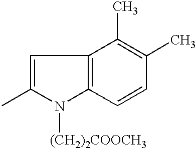 | 134 |
| 409 | 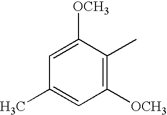 | 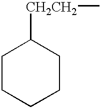 | 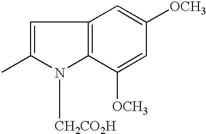 | 185 (HCl) |
| 410 | 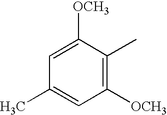 | 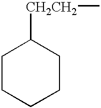 | 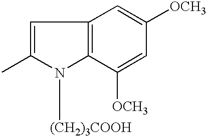 | 144 (HCl) |
| 411 | 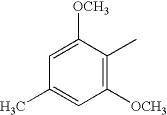 | 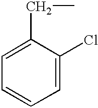 | 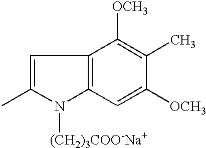 | 178 (Na salt) |
| 412 | 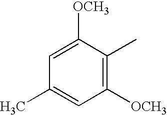 | 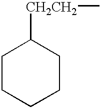 | 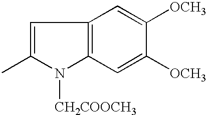 | 207 |
| 413 | 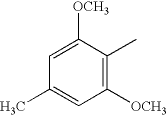 | 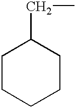 | 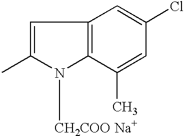 | 191 (Na salt) |
| 414 | 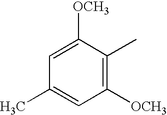 | 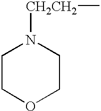 | 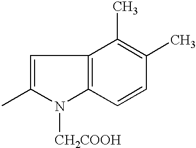 | 228 (2HCl) |
| 415 | 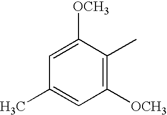 | 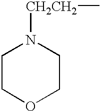 | 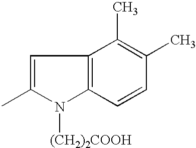 | 203 (2HCl) |
| 416 | 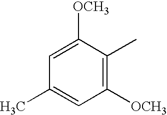 | 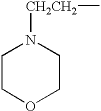 | 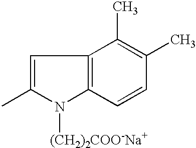 | 290 (Na salt) |
| 417 | 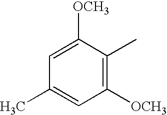 | 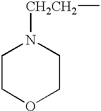 | 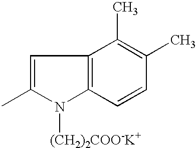 | 257 (K salt) |
| 418 | 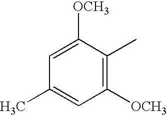 | 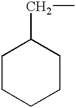 | 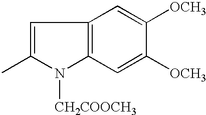 | 228 |
| 419 | 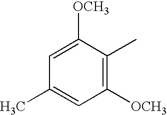 | 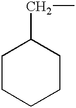 | 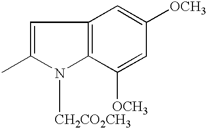 | 217 |
| 420 | 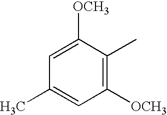 | 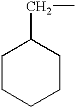 | 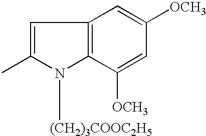 | 168 |
| 421 | 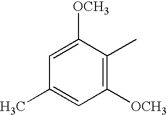 | 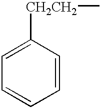 | 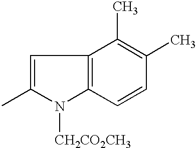 | 113 |
| 422 | 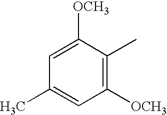 | 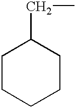 | 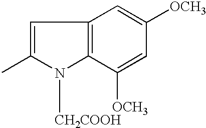 | 201 (HCl) |
| 423 | 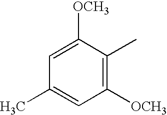 | 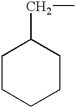 | 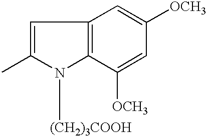 | 146 (HCl) |
| 424 | 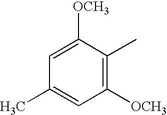 | 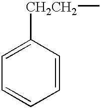 | 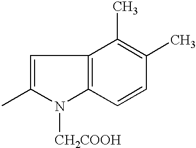 | 198 (HCl) |
| 425 | 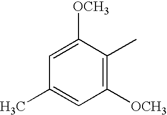 | 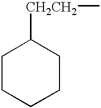 | 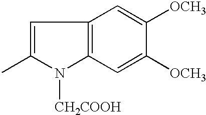 | 167 |
| 426 | 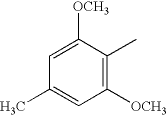 | 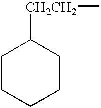 | 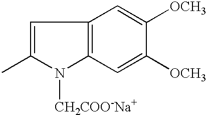 | 244 (Na salt) |
| 427 | 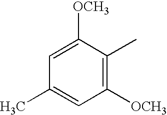 | 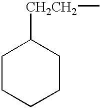 | 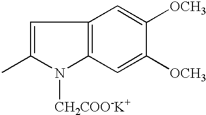 | 245 (K salt) |
| 428 | 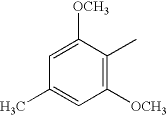 | 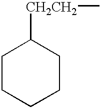 | 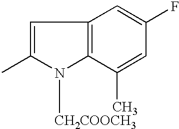 | 151 |
| 429 | 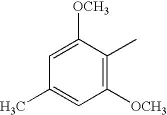 | 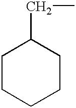 | 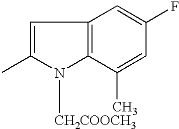 | 157 |
| 430 | 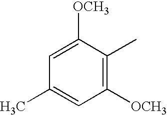 | 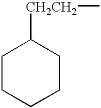 | 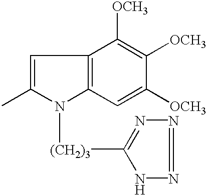 | 205 |
| 431 | 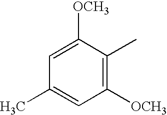 | 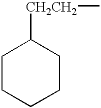 | 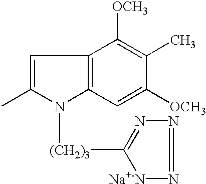 | 248 (Na salt) |
| 432 | 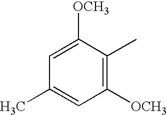 | 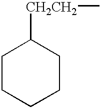 | 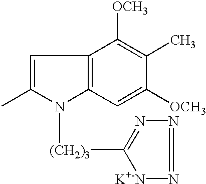 | 240 (Na salt) |
| 433 | 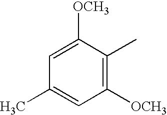 | 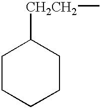 | 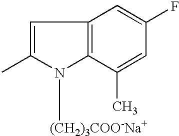 | 144 (Na salt) |
| 434 | 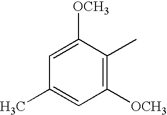 | 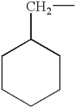 | 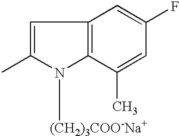 | 220 (Na salt) |
| 435 | 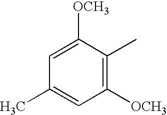 | 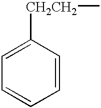 | 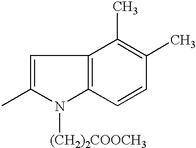 | 108 |
| 436 | 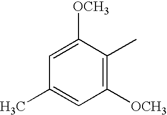 | 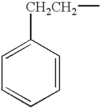 | 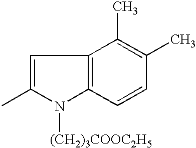 | 77 |
| 437 | 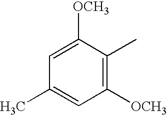 | 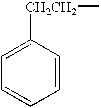 | 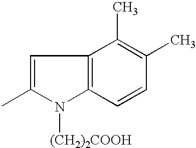 | 270 (HCl) |
| 438 | 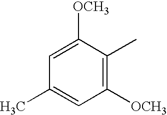 | 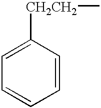 | 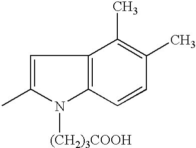 | 278 (HCl) |
| 439 | 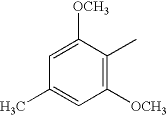 | 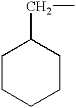 | 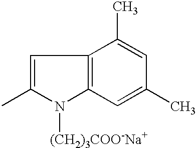 | 179 (Na salt) |
| 440 | 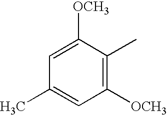 | 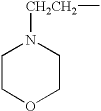 | 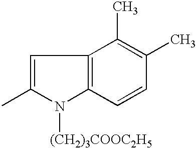 | 167 (HCl) |
| 441 | 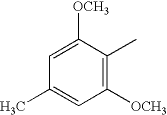 | 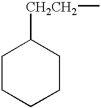 | 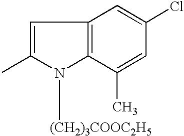 | 164 |
| 442 | 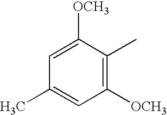 | 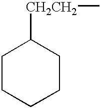 | 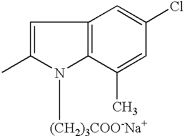 | 150 (Na salt) |
| 443 | 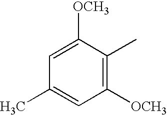 | 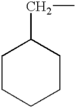 | 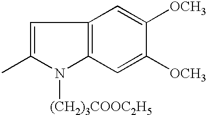 | 113 |
| 444 | 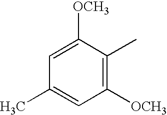 | 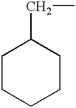 | 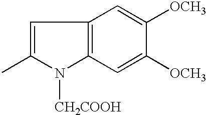 | 185 |
| 445 | 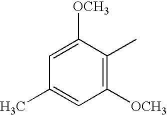 | 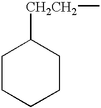 | 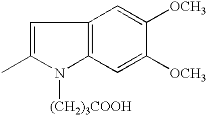 | 209 |
| 446 | 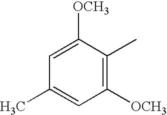 | 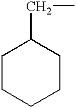 | 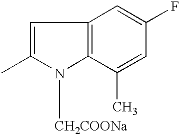 | 295 (Na salt) |
| 447 | 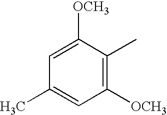 | 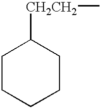 | 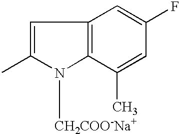 | 221 (Na salt) |
| 448 | 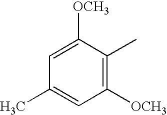 | 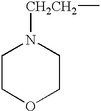 | 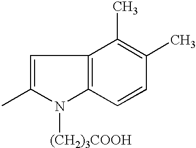 | 190 |
| 449 | 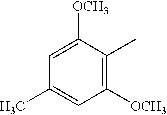 | 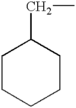 | 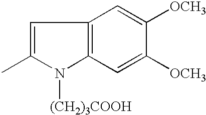 | 246 |
| 450 | 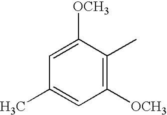 | 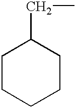 | 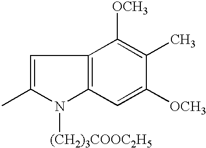 | 196 |
| 451 | 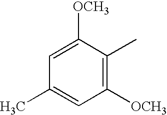 | 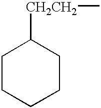 | 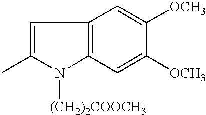 | 139 |
| 452 | 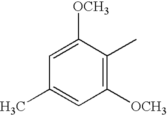 | 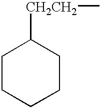 | 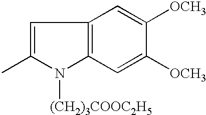 | 109 |
| 453 | 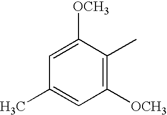 | 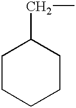 | 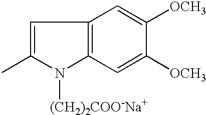 | 217 (Na salt) |
| 454 | 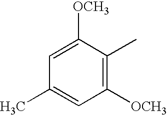 | 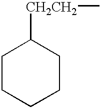 | 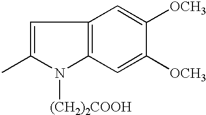 | 245 |
| 455 | 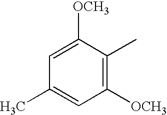 | 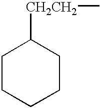 | 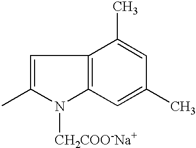 | 238 (Na salt) |
| 456 | 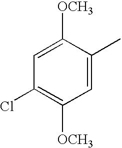 | 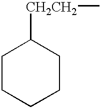 | 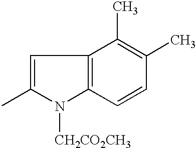 | 173 |
| 457 | 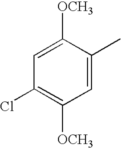 | 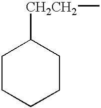 | 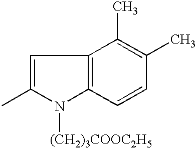 | 169 |
| 458 | 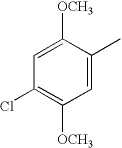 | 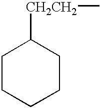 | 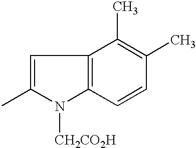 | 164 (HCl) |
| 459 | 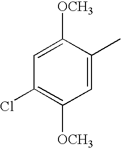 | 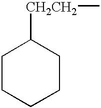 | 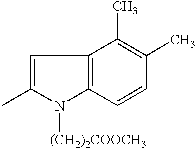 | 116 |
| 460 | 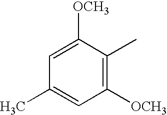 | 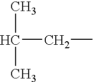 | 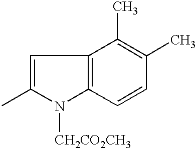 | 243 |
| 461 | 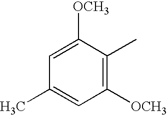 | 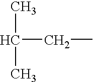 | 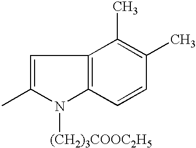 | 159 |
| 462 | 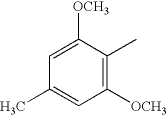 | 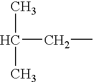 | 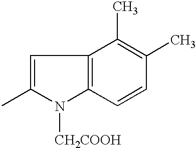 | 227 |
| 463 | 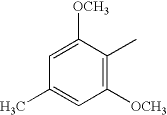 | 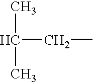 | 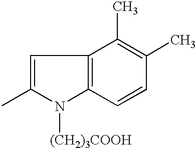 | 150 |
| 464 | 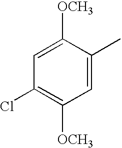 | 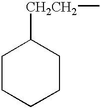 | 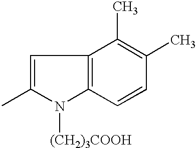 | 208 (HCl) |
| 465 | 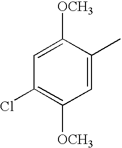 | 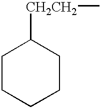 | 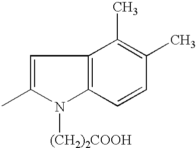 | 254 |
| 466 | 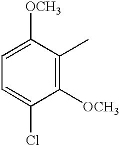 | 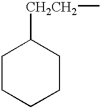 | 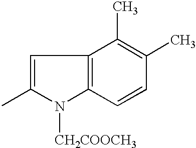 | 108 |
| 467 | 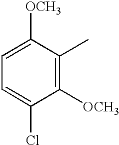 | 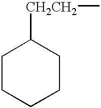 | 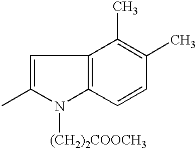 | 91 |
| 468 | 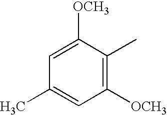 | 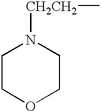 | 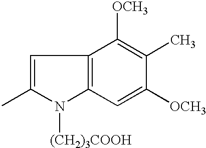 | 139 |
| 469 | 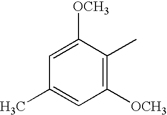 | 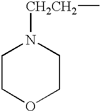 | 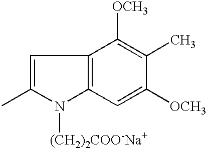 | 265 (Na salt) |
| 470 | 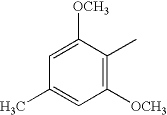 | 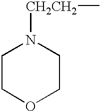 | 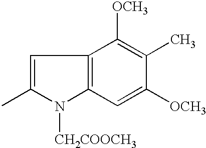 | 188 |
| 471 | 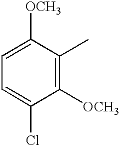 | 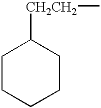 | 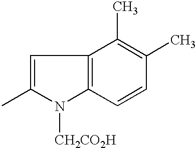 | 190 (HCl) |
| 472 | 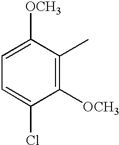 | 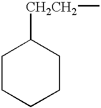 | 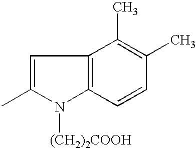 | 243 (HCl) |
| 473 | 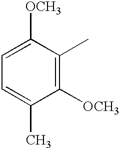 | 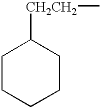 | 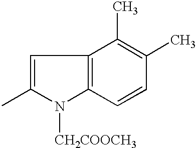 | 98 |
| 474 | 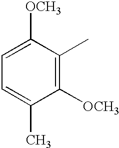 | 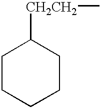 | 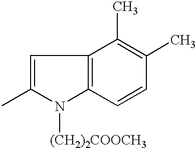 | 86 |
| 475 | 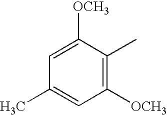 | 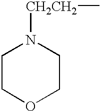 | 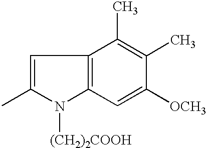 | 275 |
| 476 | 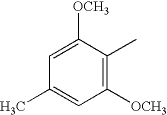 | 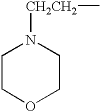 | 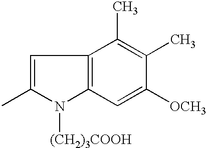 | 175 |
| 477 | 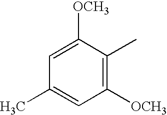 | 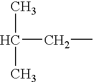 | 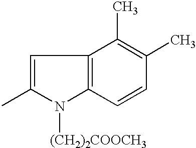 | 205 |
| 478 | 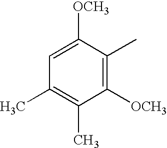 | 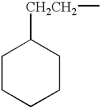 | 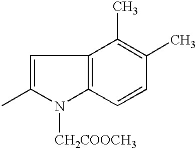 | 132 |
| 479 | 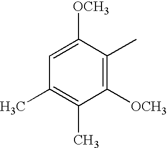 | 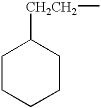 | 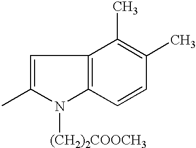 | 83 |
| 480 | 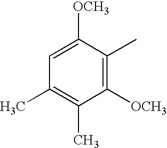 | 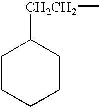 | 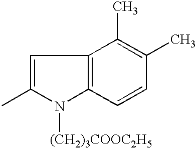 | 97 |
| 481 | 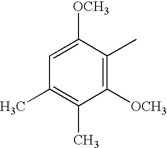 | 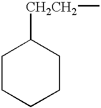 | 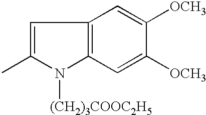 | 82 |
| 482 | 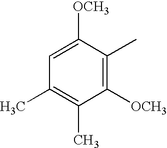 | 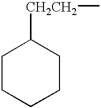 | 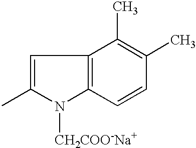 | 274 (Na salt) |
| 483 | 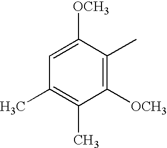 | 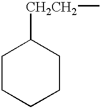 | 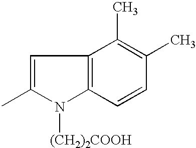 | 271 |
| 484 | 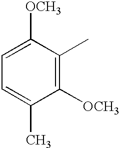 | 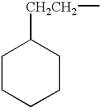 | 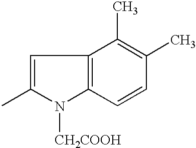 | 237 (HCl) |
| 485 | 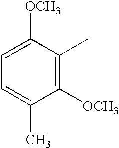 | 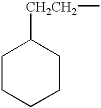 | 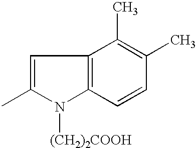 | 144 (HCl) |
| 486 | 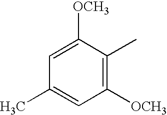 | 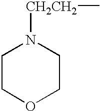 | 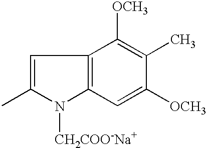 | 228 (HCl and Na salt) |
| 487 | 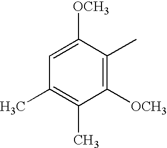 | 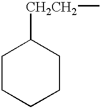 | 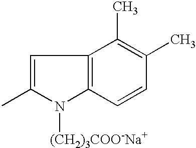 | 168 (Na salt) |
| 488 | 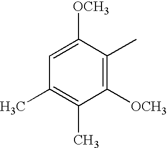 | 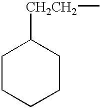 | 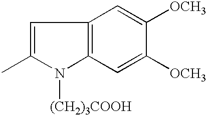 | 138 |
| 489 | 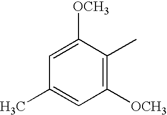 | 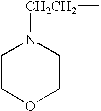 | 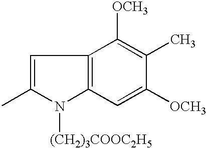 | 124 |
| 490 | 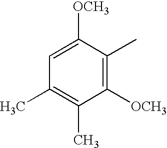 | 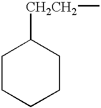 | 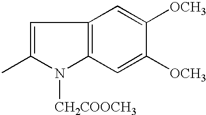 | 138 |
| 491 | 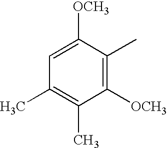 | 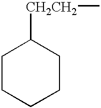 | 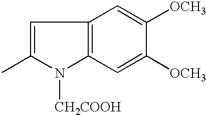 | 224 |
| 492 | 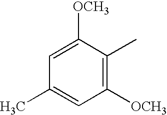 | 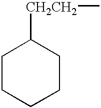 | 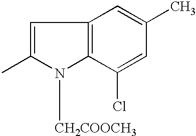 | 197 |
| 493 | 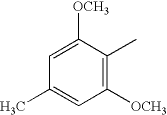 | 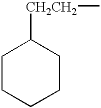 | 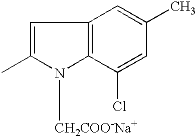 | 210 (Na salt) |
| 494 | 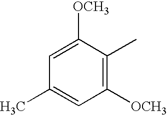 | 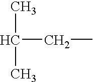 | 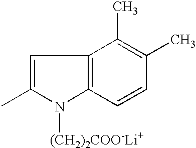 | 274 (Li salt) |
| 495 | 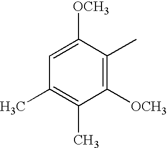 | 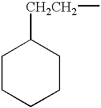 | 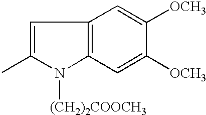 | 99 |
| 496 | 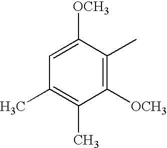 | 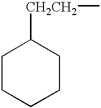 | 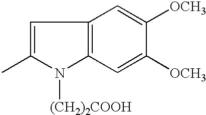 | 248 |
| 497 | 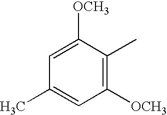 | 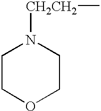 | 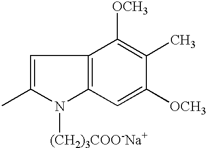 | >300 (Na salt) |
| 498 | 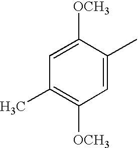 | 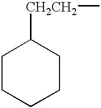 | 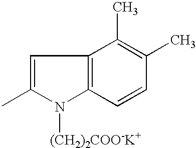 | 148 (K salt) |
| 499 | 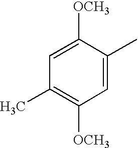 | 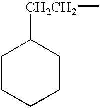 | 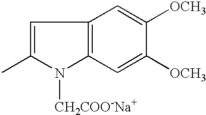 | 226 (Na salt) |
| 500 | 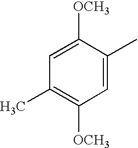 | 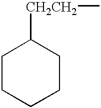 | 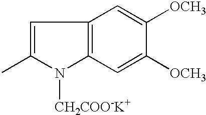 | 139 (K salt) |
| 501 | 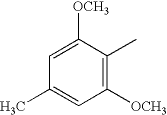 | 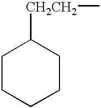 | 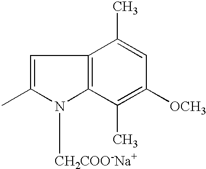 | 190 (Na salt) |
| 502 | 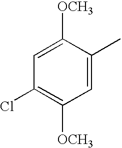 | 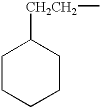 | 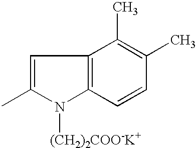 | 237 (K salt) |
| 503 | 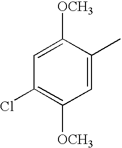 | 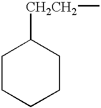 | 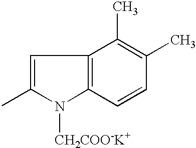 | 230 (K salt) |
| 504 | 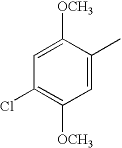 | 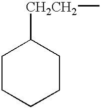 | 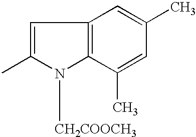 | 208 |
| 505 | 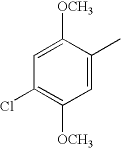 | 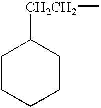 | 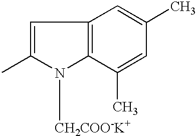 | 202 (K salt) |
| 506 | 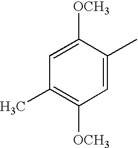 | 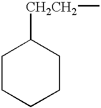 | 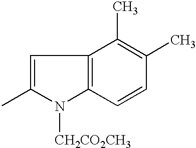 | 109 |
| 507 | 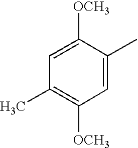 | 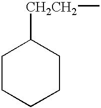 | 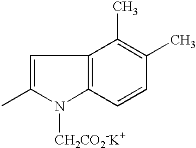 | 181 (K salt) |
| 508 | 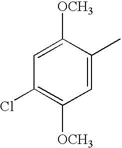 | 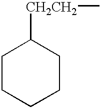 | 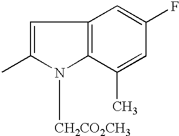 | 117 |
| 509 | 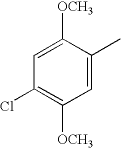 | 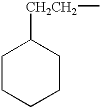 | 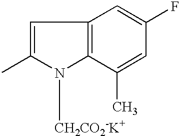 | 225 (K salt) |
| 510 | 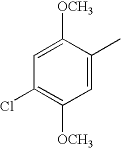 | 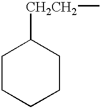 | 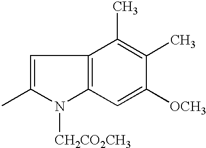 | 178 |
| 511 | 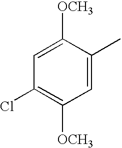 | 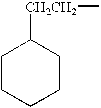 | 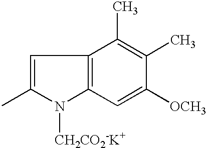 | 254 (K salt) |

### EXAMPLE 512

### 2-{N-[5-(4-chloro-2,5-dimethoxyphenyl)-1-(2-cyclohexylethyl)-1H-1,2,4-triazol-3-yl]carbamoyle}-4,5-dimethyl-1-[3-(2H-1,2,3,4-tetrazol-5-yl)-propyl]-1H-indole

Step 1: 4-[2-({[1-(2-cyclohexylethyl)-5-(2,5-dimethoxy-4-chlorophenyl)-1H-1,2,4-triazol-3-yl]amino}carbamoyl)-4,5-dimethyl-1H-1-indolyl]butyronitrile

1 ml of pyridine (0.013 mol) and 0.21 ml (0.0029 mol) of thionyl chloride are successively added to 15 ml of dichloromethane. After 15 minutes at 0° C., 0.615 g of 4,5-dimethyl-1-(3-cyanopropyl)-1H-2-indolecarboxylic acid (0.0024 mol) and then 0.9 g of 1-(2-cyclohexylethyl)-5-(2,5-dimethoxy-4-chlorophenyl)-1H-1,2,4-triazole-3-amine hydrochloride are introduced. The reaction mixture is maintained for 18 hours at room temperature, after which an acidic washing and a basic washing are carried out. The organic phase is dried over anhydrous sodium sulphate and concentrated under reduced pressure. The oily residue is chromatographed on a column of silica gel, eluting with a 99.5/1.5 (v/v) mixture to give a white powder; m.p.=178° C.; yield=87%.

Step 2: 2-{N-[5-(4-chloro-2,5-dimethoxyphenyl)-1-(2 cyclohexylethyl)-1H-1,2,4-triazol-3-yl]carbamoyle}-4,5-dimethyl-1-[3-(2H-1,2,3,4-tetrazol-5-yl)propyl]-1H-indole

0.5 ml of azidotrimethylsilane and 0.030 g of dibutyltin oxide are added to 0.720 g (0.0012 mol) of 4-[2-({[1-(2-cyclohexylethyl)-5-(2,5-dimethoxy-4-chlorophenyl)-1H-1,2,4-triazol-3-yl]amino}carbonyl)-4,5-dimethyl-1H-1-indolyl]butyronitrile dissolved in 15 ml of tetrahydrofuran and the mixture is refluxed for 18 hours. The reaction mixture is allowed to cool to room temperature, the tetrahydrofuran is removed under reduced pressure and the residue is chromatographed on a column of silica gel, eluting with a 95/5 (v/v) dichloromethane/methanol mixture. A white solid is obtained; m.p.=233° C., yield=78%.

This procedure described for Example 512 is also used for Examples 303, 304, 316, 317, 356, 357, 361, 362, 363, 368, 369, 392, 394, 395, 430, 431 and 432.

The potassium and sodium salts of these compounds are obtained in acetonitrile by addition of one equivalent of base at room temperature, followed by evaporation of the solvent under reduced pressure and then drying.

1. Compound of formula: 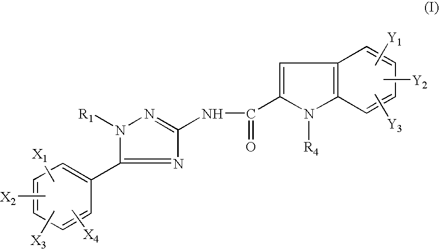
in which:
R₁ represents a (C₂-C₆)alkyl; a group —(CH₂)ₙ-G with n ranging from 0 to 5 and G representing a non-aromatic C₃-C₁₃ mono- or polycyclic hydrocarbon group optionally substituted with one or more (C₁-C₃) alkyl; a phenyl(C₁-C₃) alkyl in which the phenyl group is optionally substituted one or more times with a halogen, with a (C₁-C₃)alkyl or with a (C₁-C₃)alkoxy; a group —(CH₂)ₙNR₂R₃ in which n represents an integer from 1 to 6 and R₂ and R₃, which may be identical or different, represent a (C₁-C₃)alkyl or constitute, with the nitrogen atom to which they are attached, a morpholino, piperidino, pyrrolidinyl or piperazinyl group;
X₁, X₂, X₃ or X₄ each independently represents a hydrogen or halogen atom, a (C₁-C₆)alkyl, a (C₁-C₃)alkoxy or a trifluoromethyl; it being understood that only one from among X₁, X₂, X₃ and X₄ possibly represents a hydrogen atom;
R₄ represents hydrogen, a group —(CH₂)ₙCOOR₅ in which n is as defined above and R₅ represents a hydrogen atom, a (C₁-C₆) alkyl or a (C₆-C₁₀) aryl-(C₁-C₆)alkyl; a (C₁-C₆)alkyl; a group —(CH₂)ₙOR₅ or a group —(CH₂)ₙNR₂R₃ in which n, R₂, R₃ and R₅ are as defined above; a group —(CH₂)ₙ-tetrazolyl in which n is as defined above, or R₄ represents one of these: groups in the form of an alkali-metal or alkaline-earth metal salt;
Y₁, Y₂ and Y₃ independently represent a hydrogen, a halogen, a (C₁-C₃)alkyl, a (C₁-C₃)alkoxy, a nitro, cyano, (C₁-C₆)acylamino, carbamoyl, trifluoromethyl, a group COOR₆ in which R₆ represents hydrogen, or (C₁-C₃) alkyl;
or one of the salts or solvates thereof.

2. Compound of formula (I) according to claim 1, in which R₁, R₄, X₁, X₂, X₃ and X₄ are as defined in claim 1 and Y₁, Y₂ and Y₃ represent hydrogen; a salt or solvate thereof.

3. Compound of formula (I) according to claim 1, in which R₁ and R₄ are as defined in claim 1, Y₁, Y₂ and Y₃ represent hydrogen; and 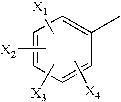
represents 2,6-dimethoxy-4-methylphenyl;
a salt or solvate thereof.

4. Compound of formula (I) according to claim 1, in which R₁, R₄, Y₁, Y₂ and Y₃ are as defined in claim 1, and 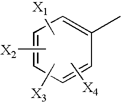
represents 2,6-dimethoxy-4-methylphenyl;
a salt or solvate thereof.

5. Compound of formula (I) according to claim 1, in which R₁, R₄, Y₁, Y₂ and Y₃ are as defined in claim 1, and 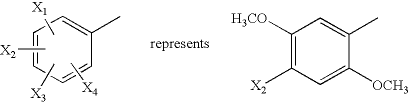
X₂ representing methyl or a chlorine atom;
a salt or solvate thereof.

6. Compound of formula: 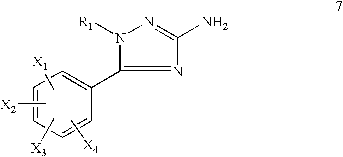
in which R₁, X₁, X₂, X₃ and X₄ are as defined for (I) in claim 1.

7. Process for the preparation of a compound of formula (I) according to any one of claims 1 to 5, comprising the step consisting in reacting an aminotriazole of formula: 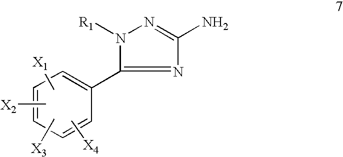
in which R₁, X₁, X₂, X₃ and X₄ are as defined for (I) in claim 1, with an indolecarboxylic acid derivative of formula 8: 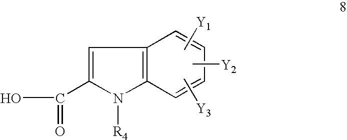
in which R₄, Y₁, Y₂ and Y₃ are as defined for (I) in claim 1, in order to obtain the compounds of formula (I),
a salt or solvate thereof.

8. Process for the preparation of a compound of formula (I) according to any one of claims 1 to 5, comprising the reaction of an aminotriazole of formula: 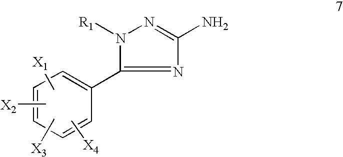
in which R₁, X₁, X₂, X₃ and X₄ are as defined for (I)
either with an indolecarboxylic acid derivative of formula: 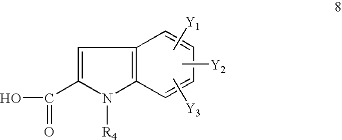
in which R₄, Y₁, Y₂ and Y₃ are as defined above for (I);
or with an indolecarboxylic acid derivative of formula: 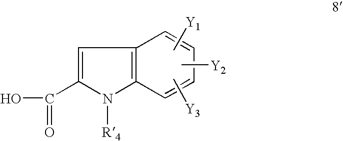
in which Y₁, Y₂ and Y₃ are as defined above for (I) and R′₄ is a precursor group of R₄, in which case the compound of formula: 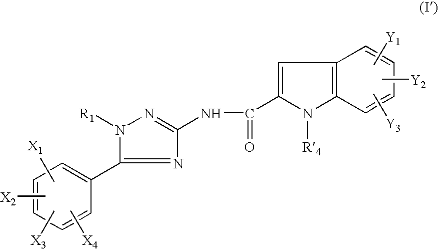
in which R₁, X₁, X₂, X₃, X₄, Y₁, Y₂ and Y₃ are as defined for (I) and R′₄ is a precursor group of R₄, R₄ being defined for (I), is formed as an intermediate.

9. Pharmaceutical composition containing, as active principle, a compound of formula (I) according to claim 1, or one of the pharmaceutically acceptable salts thereof.

10. Pharmaceutical composition containing, as active principle, a compound according to claim 2, or one of the pharmaceutically acceptable salts thereof.

11. Pharmaceutical composition containing, as active principle, a compound according to claim 3, or one of the pharmaceutically acceptable salts thereof.

12. Pharmaceutical composition containing, as active principle, a compound according to claim 4, or one of the pharmaceutically acceptable salts thereof.

13. Pharmaceutical composition containing, as active principle, a compound according to claim 5, or one of the pharmaceutically acceptable salts thereof.

14. Use of a compound according to any one of claims 1 to 5 for the preparation of medicines intended to treat eating behaviour disorders and obesity and to reduce the intake of food.

15. Use of a compound according to any one of claims 1 to 5, for the preparation of medicines intended to treat tardive dyskinesia.

16. Use of a compound according to any one of claims 1 to 5 for the preparation of medicines intended to treat disorders of the gastrointestinal sphere.
